(19) United States
(12) Patent Application Publication
(10) Pub. No.: US 2013/0040934 A1
(43) Pub. Date: Feb. 14, 2013

(54) SPIROCYCLIC COMPOUNDS AND THEIR USE AS THERAPEUTIC AGENTS AND DIAGNOSTIC PROBES

(71) Applicants: Vladimir Cmiljanovic, Basel (CH); Natasa Cmiljanovic, Basel (CH); Bernd Giese, Fribourg (CH); Matthias Wymann, Bern (CH)

(72) Inventors: Vladimir Cmiljanovic, Basel (CH); Natasa Cmiljanovic, Basel (CH); Bernd Giese, Fribourg (CH); Matthias Wymann, Bern (CH)

(21) Appl. No.: 13/635,016

(22) PCT Filed: Mar. 11, 2011
(86) PCT No.: PCT/IB2011/051047

§ 371 Date: Sep. 14, 2012

(30) Foreign Application Priority Data
Mar. 15, 2010 (GB) 1004200.0

(51) Int. Cl. A61K 31/53; C12N 9/99; A61P 35/00; C07D 519/00; A61K 31/506; C07D 487/10; C07D 491/107; A61K 31/5377

(52) U.S. Cl. 514/210.21; 544/198; 435/184; 544/70; 544/230

(57) ABSTRACT

The invention relates to new triazines (G=Q=U are N), pyrimidines (two out of G, Q and U are N), and pyridopyrimidines (one of G and U together with R2 forms an anullated pyridine ring) of formula (I) carrying a spirocyclic substituent, wherein E¹ is CR⁴ or N; X¹ is CHR⁴, CH₂CH₂, NR⁴, NR4→0, or O; and the other substituents are as defined in the specification. The compounds inhibit phosphoinositide 3-kinase (PI3K), mammalian target of rapamycin (mTOR), DNA-PK and ATM kinase, and may be used as therapeutic agents or diagnostic probes. The invention also relates to methods of using the compounds for treatment of associated pathological conditions.

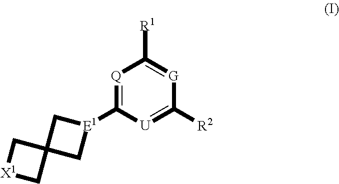

### FIELD OF THE INVENTION

The invention relates to new triazines and pyrimidines carrying a spirocyclic substituent, which inhibit phosphoinositide 3-kinase (PI3K), mammalian target of rapamycin (mTOR), DNA-PK and ATM kinase, and pharmaceutically acceptable salts thereof. The invention also relates to methods of using the compounds for treatment of associated pathological conditions.

### BACKGROUND OF THE INVENTION

Protein kinases participate in the signaling events which control the activation, growth, differentiation, survival and migration of cells in response to extracellular mediators or stimuli including growth factors, cytokines or chemokines. In general, these kinases are classified in two groups, those that preferentially phosphorylate tyrosine residues and those that preferentially phosphorylate serine and/or threonine residues. The tyrosine kinases include membrane-spanning growth factor receptors, for example the epidermal growth factor receptor (EGFR) and cytosolic non-receptor kinases including Src family kinases, the Syk family kinases and the Tec family kinases.

Inappropriately high protein kinase activity is involved in many diseases including cancer, metabolic diseases, immunological diseases and inflammatory disorders. This can be caused either directly or indirectly by the failure of control mechanisms due to mutation, overexpression or inappropriate activation of the enzyme.

Phosphoinositide 3-kinases (PI3Ks) were early on identified as lipid kinases associated with viral oncogens [Whitman et al., Nature 315:239-242 (1985); Sugimoto et al., Proc. Natl. Acad. Sci. 81:2117-2121 (1984); Macara et al., Proc. Natl. Acad. Sci. 81:2728-2732 (1984)], and for the last 20 years, the connection between cancer and PI3K has been further substantiated [Cully et al., Nat. Rev., Cancer 6:184-192 (2006); Wymann et al., Curr. Opin. Cell Biol. 17:141-149 (2005); Vivanco et al., Nat. Rev. Cancer 2:489-501 (2002)]. PI3Ks have since been recognized to modulate a wide range of cellular activities, and to be central to the growth and metabolic control. Genetically modified mice targeting the PI3K pathway, and the elucidation of human hereditary disease like Cowden's syndrome, tuberous sclerosis, ataxia telangiectasia, X-linked myotubular myopathy and Charcot-Marie-Tooth neuropathy, have provided further insight in the cellular and systemic role of phosphoinositide signaling. Deregulation of phosphoinositide levels, and in particular the product of class I PI3Ks, PtdIns (3,4,5)P3, is involved in the pathogenesis of cancer, chronic inflammation, allergy, metabolic disease, diabetes and cardiovascular problems.

The PI3 kinase/Akt/PTEN pathway is an attractive target for cancer drug development since such agents would be expected to inhibit proliferation, reverse the repression of apoptosis and surmount resistance to cytotoxic agents in cancer cells. PI3 kinase inhibitors have been reported [see notably Marone et al., Biochimica et Biophysica Acta 1784:159-185 (2008)].

1,3,5-triazine and pyrimidine derivatives as pharmaceuticals have been made with respect to antitumor, anti-inflammatory, analgesic and antispasmodic activities. Especially, hexamethylmelamine or altretamine (HMM or N²,N²,N⁴,N⁴,N⁶, N⁶-hexamethyl-1,3,5-triazine-2,4,6-triamine) is well-known, which has been developed as analogue of antitumor agent triethylenemelamine (TEM); HMM acts as a prodrug of hydroxymethylpentamethylmelamine (HMPMM: metabolically active type of HMM) [Johnson et al., Cancer, 42:2157-2161 (1978)]. HMM has been marketed in Europe under the indications for the treatment of ovarian and small cell lung cancers.

Certain triazine compounds are known to have PI3K and/or mTOR inhibitor activity and inhibit the growth of cancer cells [WO 02/088112, WO 2009/905138, WO 2009/143313, WO 2009/143317]. The triazine compound ZSTK474 (Zenyaku Kogyo) is the first orally administered triazine compound highly active against PI3Ks that displayed potent antitumor activity against human cancer xenografts in mice, without evidence of critical toxicity [Yaguchi et al., Journal of the National Cancer Institute, 98:545-556, (2006)]. ZSTK474 is an ATP-competitive inhibitor of class I phosphatidyl-inositol 3-kinase isoforms [Kong et al., Cancer Sci, 98:1638-1642 (2007)].

Certain pyrimidine compounds are known to have PI3K and/or mTOR inhibitor activity and inhibit the growth of cancer cells [WO 2006/090167, WO 2007/066103, WO 2008/032033, WO 2008/032072, WO 2007/084786, WO 2008/098058].

In order to expand the antitumor spectrum and to increase antitumor activities of such compounds, active against PI3Ks and/or mTOR, the inventors carried out intensive studies on triazine-, pyrimidine- and pyridine-based derivatives. They thus prepared new heterocyclic compounds represented by the formulas (I) to (V) which exhibit strong biological activity against lipid kinases. In comparison with the PI3K inhibitors of the prior art the inhibitors of the invention differ in the insertion of a heteroatom containing spirocyclic group making the novel molecules superior regarding their pharmacological properties.

### SUMMARY OF THE INVENTION

The invention relates to compounds of formula (I)

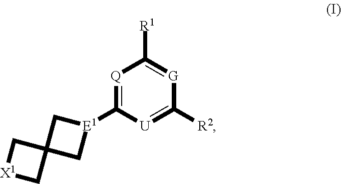

wherein

G is CH or N, Q is CH or N, and U is CH or N, with the proviso that at least two of G, Q and U are N, or one of G and U together with R² forms an anullated pyridine ring further substituted by R³, and the other one of G and U is N and Q is N;

E¹ and E² are, independently of each other, CR⁴ or N;

X¹ and X² are, independently of each other, CHR⁴, CH₂CH₂, NR⁴, NR⁴→O, or O;

R¹ is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, a linker carrying a reactive group and/or a tag, or

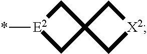

R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;

R³ is optionally substituted amino, optionally substituted C₆-C₂₀-aryl, or optionally substituted C₁-C₁₉-heteroaryl;

R⁴ is hydrogen, C₁-C₆-alkyl, C₁-C₆-acyl, C₁-C₆-acylamino-C₁-C₆-alkyl, a reactive group or a linker carrying a reactive group and/or a tag;

and tautomers, prodrugs, metabolites, solvates and pharmaceutically acceptable salts thereof.

Another aspect of the invention provides a pharmaceutical composition comprising a compound of formula (I) as defined hereinbefore and a pharmaceutically acceptable carrier. The pharmaceutical composition may further comprise one or more additional therapeutic agents selected from chemotherapeutic agents, anti-proliferative agents, anti-inflammatory agents, immunomodulatory agents, neurotropic factors, agents for treating blood disorders, agents for treating diabetes, and agents for treating immunodeficiency disorders.

Another aspect of the invention provides methods of inhibiting PI3 kinase activity, comprising contacting a PI3 kinase with an effective inhibitory amount of a compound of formula (I) as defined hereinbefore.

Another aspect of the invention provides methods of preventing or treating a disease or disorder modulated by PI3 kinases and/or mTOR, comprising administering to a mammal in need of such treatment an effective amount of a compound of formula (I) as defined hereinbefore. Examples of such diseases, conditions and disorders include, but are not limited to, hyperproliferative disorders (e.g., cancer, including melanoma and other cancers of the skin), neurodegeneration, cardiac hypertrophy, pain, migraine, neuro-traumatic diseases, stroke, diabetes, hepatomegaly, cardiovascular disease, Alzheimer's disease, cystic fibrosis, autoimmune diseases, atherosclerosis, restenosis, psoriasis, allergis disorders, inflammation, neurological disorders, hormone-related diseases, conditions associated with organ transplantation, immunodeficiency disorders, destructive bone disorders, hyperproliferative disorders, infectious diseases, conditions associated with cell death, thrombin-induced platelet aggregation, chronic myelogenous leukaemia (CML), liver disease, pathologic immune conditions involving T cell activation, and CNS disorders.

Another aspect of the invention provides methods of preventing or treating a hyperproliferative disorder, comprising administering to a mammal in need of such treatment an effective amount of a compound of formula (I) as defined hereinbefore, alone or in combination with one or more additional compounds having anti-hyperproliferative properties.

An additional aspect of the invention is the use of a compound of this invention in the preparation of a medicament for the treatment or prevention of a disease or condition modulated by PI3 kinase and/or mTOR in a mammal.

Another aspect of the invention includes kits comprising a compound of formula (I) as defined hereinbefore, a container, and optionally a package insert or label indicating a treatment.

Another aspect of the invention includes methods of preparing, methods of separating, and methods of purifying compounds of formula (I) as defined hereinbefore.

Another aspect of the invention includes novel intermediates useful for preparing compounds of formula (I) as defined hereinbefore.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar to and equivalent to those described herein, which could be used in the practice of the present invention. The present invention is in no way limited to the methods and materials herein described.

The term “alkyl” as used herein refers to a saturated linear or branched-chain monovalent hydrocarbon radical of one to twelve carbon atoms (C₁-C₁₂), wherein the alkyl radical may be optionally substituted independently with one or more substituents described below. Preferably, alkyl has one to eight carbon atoms (C₁-C₈), or more preferably one to six carbon atoms (C₁-C₆), in particular one to four carbon atoms (C₁-C₄). Examples of alkyl groups include, but are not limited to, methyl, ethyl, n-propyl, iso-propyl, n-butyl, iso-butyl, sec-butyl, tert-butyl, n-pentyl, iso-pentyl, n-hexyl, n-heptyl, n-octyl, and the like.

The term “alkenyl” refers to linear or branched-chain monovalent hydrocarbon radical of two to eight carbon atoms (C₂-C₈) with at least one site of unsaturation, i.e., a carbon-carbon sp2 double bond, wherein the alkenyl radical may be optionally substituted independently with one or more substituents described herein, and includes radicals having “cis” and “trans” orientations, or alternatively, “E” and “Z” orientations. Preferably, alkenyl has two to six carbon atoms (C₂-C₆), in particular two to four carbon atoms (C₂-C₄). Examples include, but are not limited to, vinyl, allyl, and the like.

The term “alkynyl” refers to a linear or branched monovalent hydrocarbon radical of two to eight carbon atoms (C₂-C₈) with at least one site of unsaturation, i.e., a carbon-carbon sp triple bond, wherein the alkynyl radical may be optionally substituted independently with one or more substituents described herein. Preferably, alkynyl has two to six carbon atoms (C₂-C₆), in particular two to four carbon atoms (C₂-C₄). Examples include, but are not limited to, ethynyl, propargyl, and the like.

The term “halogen” (or halo) preferably represents chloro or fluoro, but may also be bromo or iodo.

The terms “carbocycle”, “carbocyclyl”, “carbocyclic ring” and “cycloalkyl” refer to a monovalent non-aromatic, saturated or partially unsaturated ring having 3 to 12 carbon atoms (C₃-C₁₂) as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, or as bridged systems such as bicyclo[2.2.1]heptane, bicyclo[2.2.2]-octane, bicylco[3.3.1]nonane and bicyclo[3.2.2]nonane. Examples of monocyclic carbo-cycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The term “aryl” means a monovalent aromatic hydrocarbon radical of 6-20 carbon atoms (C₆-C₂₀) derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated, or aromatic carbocyclic ring. Typical aryl groups include, but are not limited to, radicals derived from benzene(phenyl), substituted benzenes, naphthalene, anthracene, biphenyl, indenyl, indanyl, 1,2-dihydronapthalene, 1,2,3,4-tetra-hydronaphthalene, and the like. Aryl groups are optionally substituted independently with one or more substituents described herein.

The terms “heterocycle”, “heterocyclyl” and “heterocyclic ring” are used inter-changeably herein and refer to a saturated or a partially unsaturated (i.e., having one or more double and/or triple bonds within the ring) carbocyclic radical of 3 to 20 ring atoms in which at least one ring atom is a heteroatom selected from nitrogen, oxygen, phosphorus and sulphur, the remaining ring atoms being carbon atoms, where one or more ring atoms are optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example, a bicyclo [4,5], [5,5], [5,6], or [6,6] system. “Heterocyclyl” also includes radicals wherein heterocycle radicals are fused with a saturated or partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydro-pyranyl, dihydrothienyl, dihydrofuranyl, pyrazolidinylimidazolinyl, imidazolidinyl, 3-aza-bicyclo[3.1.0]hexyl, 3-azabicyclo[4.1.0]heptyl, azabicyclo[2.2.2]hexyl, 3H-indolyl, and quinolizinyl. Spiro moieties are also included within the scope of this definition. Examples of a heterocyclic group wherein 1 or 2 ring carbon atoms are substituted by oxo are pyrimidinonyl and 1,1-dioxo-thiomorpholinyl. The heterocycle groups herein are optionally substituted independently with one or more substituents described herein.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulphur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, oxazolyl, isoxazolyl, thiazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzooxazolyl, quinazolinyl, quinoxalinyl, naphthyridinyl, and furopyridinyl. Heteroaryl groups are optionally substituted independently with one or more substituents described herein.

The heterocyclyl or heteroaryl groups may be carbon-linked or nitrogen-linked where such is possible. By way of example and not limitation, carbon-linked heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydro-pyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline, or position 1, 3, 4, 5, 6, 7, or 8 of an isoquinoline.

By way of example and not limitation, nitrogen-linked heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole, position 2 of an isoindole or isoindoline, position 4 of a morpholine, and position 9 of a carbazole or β-carboline.

The term “acyl” as used herein refers to an alkyl, alkenyl, alkynyl, carbocyclyl, aryl, heterocyclyl, or heteroaryl group connected to carbonyl, sulfonyl, oxycarbonyl or amino-carbonyl. Acyl has one to twenty carbon atoms (C₁-C₂₀), and may be optionally substituted independently with one or more substituents described above and below. Preferably, acyl has one to twelve carbon atoms (C₁-C₁₂), or more preferably one to eight carbon atoms (C₁-C₈), in particular one to six carbon atoms (C₁-C₆). Examples of acyl groups include, but are not limited to, formyl, acetyl, propionyl, butyryl, acryloyl, methacryloyl, 2,3-epoxy-propionyl; hydroxy-, fluoro-, chloro- or bromo-acetyl; cyclopentanecarbonyl, cyclohexane-carbonyl, benzoyl; p-amino-, p-hydroxy-, p-methoxy- or p-methylbenzoyl; 2,4-dinitro-benzoyl, 3,5-dimethoxy-4-hydroxybenzoyl, α- or β-naphthoyl, pyridin-2-, 3- or 4-ylcarbonyl, 2-aminopyridin-5-ylcarbonyl, 2-amino-4-trifluoromethylpyridin-5-ylcarbonyl, pyrimidin-2-ylcarbonyl, furylcarbonyl, thienylcarbonyl, methanesulfonyl, trifluoromethanesulfonyl, chloro- or bromomethanesulfonyl, p-toluolenesulfonyl, methoxycarbonyl, ethoxycarbonyl, benzyloxycarbonyl, methylaminocarbonyl, ethylaminocarbonyl, benzylaminocarbonyl, or pyridylaminocarbonyl.

The term “reactive group” includes, but is not limited to electrophilic reactive groups and photoreactive groups. An electrophilic reactive group is a chemical function which reacts with a nucleophile, for example with a basic nitrogen atom, a nucleophilic hydroxy group, oxy anion or a sulfur anion of an enzyme, and in general comprises a carbon-carbon double bond conjugated with a carbon-oxygen double bond or with a sulfone function, an epoxy function, or an easily displaceable halogen or sulfonate function. Particular examples of electrophilic reactive groups are acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl; fluoro-, chloro-, bromo- or iodoacetyl; chloro- or bromomethanesulfonyl, 2,2-dichloroacetyl, 2,2,2-trichloro-acetyl, methylsulfonyloxyacetyl, 2-chloropropionyl, 2,3-epoxypropionyl, (phenylthio)thio-carbonyl, 2-nitrophenoxycarbonyl, or 4-fluorophenoxycarbonyl, preferably bound to an nitrogen atom X as defined above and below. A photoreactive group is a group giving a reactive radical species on activation with light. Particular examples of photoreactive groups are azidobenzoyl, azido-tetrafluorobenzoyl, benzophenone-4-carbonyl, or 4-(3-(trifluoromethyl)-3H-diazirin-3-yl)benzoyl.

The term “linker” includes, but is not limited to, a chain of 1 to 20, preferably 2 to 6, optionally substituted methylene groups, or such chain wherein one or more methylene groups are replaced by oxygen, a carbonyloxy group, optionally substituted nitrogen, a carboxamide group, a urea group, sulphur, a disulfide group, or combinations thereof. Substituents considered are oxo (giving a carbonyl function), C₁-C₆ alkyl, a chain of 1 to 6 methylene groups giving rise to a trifunctional linker, phenyl, phenylene giving rise to a trifunctional linker, or residues of naturally occurring amino acids. Particular linkers are, e.g., a polymethylene group, a polymethylene group comprising one or two amide functions, a polyoxyethylene group, or a small peptide consisting of one to six of the naturally occurring 20 essential amino acids. The linker may be directly connected to the nucleus of formula (I) including X¹ and X², or by way of a reactive group as defined above. “A linker carrying a reactive group and/or a tag” means a linker connected to the nucleus of formula (I) including X¹ and X², carrying a reactive group or a tag at the other end of the linker, or being a trifunctional linker carrying both a reactive group and a tag or carrying two different tags. Alternatively, a linker carrying both a reactive group and a tag may be a bifunctional linker connected to a reactive group and a tag, wherein the reactive group is connected to the nucleus of formula (I) including X¹ and X².

The term “tag” includes, but is no limited to biotin, avidin, streptavidin, a fluorescent marker, a naturally occurring amino acid, or a solid phase, for example a polymeric bead or a plastic or glass slide. Examples of fluorescent markers considered are 4,4-difluoro-1,3,5,7-tetramethyl-4-bora-3a,4a-diaza-s-indacene-8-propionic acid (BODIPY® 493/503, SE),4,4-difluoro-5,7-dimethyl-4-bora-3a,4a-diaza-s-indacene-3-propionic acid (BODIPY® FL), 4,4-difluoro-5,7-dimethyl-4-bora-3a,4a-diaza-s-indacene-3-propionic acid (BODIPY® FL, SE),6-((4,4-difluoro-5,7-dimethyl-4-bora-3a,4a-diaza-s-indacene-3-propionyl)amino)-hexanoic acid (BODIPY® FL-X, SE),4,4-difluoro-5-phenyl-4-bora-3a,4a-diaza-s-indacene-3-propionic acid (BODIPY® R6G, SE),4,4-difluoro-5,7-diphenyl-4-bora-3a,4a-diaza-s-indacene-3-propionic acid (BODIPY® 530/550, SE),6-((4,4-difluoro-1,3-dimethyl-5-(4-methoxyphenyl)-4-bora-3a,4a-diaza-s-indacene-2-propionyl)amino)hexanoic acid (BODIPY® TMR-X, SE),4,4-difluoro-5-(2-thienyl)-4-bora-3a,4a-diaza-s-indacene-3-propionic acid (BODIPY® 558/568, SE),4,4-difluoro-5-styryl-4-bora-3a,4a-diaza-s-indacene-3-propionic acid (BODIPY® 564/570, SE),6-(((4-(4,4-difluoro-5-(2-thienyl)-4-bora-3a,4a-diaza-s-indacene-3-yl)phenoxy)acetyl)amino)hexanoic acid (BODIPY® TR-X, SE),6-(((4,4-difluoro-5-(2-thienyl)-4-bora-3a,4a-diaza-s-indacene-3-yl)styryloxy)acetyl)-aminohexanoic acid (BODIPY® 630/650-X, SE), Alexa Fluor® 350 carboxylic acid, 5-carboxyrhodamine 6G (5-CR 6G, SE), Rhodamine Green™ carboxylic acid, hydrochloride (5(6)-CR 110, SE), which are usually applied as succinimidyl esters for reaction with a nitrogen atom X¹ or X² or a linker containing an amine functional group.

The term “treat” and “treatment” refer to both therapeutic treatment and prophylactic or preventative measures, wherein the object is to prevent or slow down (lessen) an undesired pathological change or disorder, such as the development or spread of cancer. For purpose of this invention, beneficial or desired clinical results include, but are not limited to, alleviation of symptoms, diminishment of extent of disease, stabilizing (i.e., not worsening) the disease state, delay or slowing of disease progression, amelioration or palliation of the disease state, and partial or total remission, whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukaemia or lymphoid malignancies. More particular examples of such cancers include squamous cell cancer (e.g., epithelial squamous cell cancer), lung cancer including small-cell lung cancer, non-small-cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatome, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “prodrug” as used in this application refers to a precursor or derivative form of a compound of the invention that may be less cytotoxic to cells compared to the parent compound or drug and is capable of being enzymatically or hydrolytically activated or converted into the more active parent form. The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, and optionally substituted phenylacetamide-containing prodrugs.

A “chemotherapeutic agent” is a chemical compound useful in the treatment of cancer. Examples of known chemotherapeutic agents include trastuzumab, pertuzumab, erlotinib, bortezomib, fulvestrant, sunitib, letrozole, imatinib mesylate, finasunate, oxaliplatin, 5-fluorouracil, leucovorin, rapamycin, lapatinib, lonafarnib, sorafenib, gefitinib, AG1478, alkylating agents such as thiotepa, cyclophosphamide; alkyl sulfonates such as busulfan, improsulfan and piposulfan; aziridines such as benzodopa, carboquone, meturedopa, and uredopa; ethyleneimines and melamines including altretamine, triethylene-melamine, triethylenephosphoramide, triethylenethiophosphoramide and trimethylomelamine; acetogenins; a camptothecin (including the synthetic analog topotecan); bryostatin; callystatin; CC-1065 (including the synthetic analogs adozelesin, carzelesin and bizelesin); cryptophycins; dolastatin; duocarmycin (including the synthetic analogs KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal and calicheamicin omegal; dynemicin, including dynemicin A; biphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophillin, chromomycinis, dactinomycin, daunorubicin, detorubicin, 6-diazol-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofuran; spirogermanium; tenuazonic acid; triaziquone; trichothecenes; urethane; vindesine; dacarbazine; mannomustine; mitobronitol; mitolactol; pipobroman; gacytosine; arabinoside; taxoids, e.g., paclitaxel, albumin-engineered nanoparticle formulations of paclitaxel, and docetaxel, doxetaxel; chlorambucil; gemcitabine; 6-thioguanine; mercaptopurine; methotrexate; platinum analogs such as cisplatin and carboplatin; vinblastine; etoposide; ifosfamide; mitoxantrone; vincristine; vinorelbine; novantrone; teniposide; edatrexate; daunomycin; aminopterin; capecitabine; ibandronate; CP-11; topoisomerase inhibitor RFS 2000; difluoromethylornithine (DMFO); retinoids such as retinoic acid; and pharmaceutically acceptable salts; acids and derivatives of any of the above.

Also included in the definition of “chemotherapeutic agent” are: (i) anti-hormonal agents that act to regulate or inhibit hormone action on tumors such as anti-estrogens and selective receptor modulators (SERMs), including, for example, tamoxifen, tamoxifen citrate, raloxifene, droloxifene, and toremifine citrate; (ii) aromatase inhibitors that inhibit the enzyme aromatase, which regulates estrogen production in the adrenal glands, such as, for example, 4(5)-imidazoles, megestrol acetate; exemestane; formestanie, fadrazole, vorozole, letrozole, and anastrozole; (iii) anti-androgens such as flutamide, nilutamide; (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Raf1 and H-Ras; (vii) ribozymes such as VEGF expression inhibitors and HER2 expression inhibitors; (viii) vaccines such as gene therapy vaccines, for example, plasmid/lipid complex containing the DNA sequences encoding HLA-B7 and β2 microglobulin, or DNA sequences encoding interleukin-2, aldesleukin (rIL-2); a topoisomerase 1 inhibitor such as lurtotecane or abarelix; (ix) anti-angiogenic agents such as bevacizumab; and (x) pharmaceutically acceptable salts, acids and derivatives of any of the above.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, glycosylation, enzymatic cleveage, and combinations thereof, of the administered compound. Particular metabolites are hydroxylated compounds and glucuronides. Accordingly, the invention includes metabolites of compounds of the invention, including compounds produced by a process comprising contacting a compound of this invention with a mammal for a period of time sufficient to yield a metabolic product thereof.

A “liposome” is a small vesicle composed of various types of lipids, phospholipids and/or surfactant, which is useful for delivery of a drug (such as the PI3K and mTOR kinase inhibitors disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The term “chiral” refers to molecules, which have the property of non-identity of the mirror image, while the term “achiral” refers to molecules, which are superimposable on their mirror image.

The term “stereoisomers” refers to compounds, which have identical chemical constitution, but differ with regard to the arrangement of the atoms or groups in space.

“Diastereomer” refers to a stereoisomer with two or more centers of chirality. Diastereomers are not mirror images of one another, and they have different physical properties, e.g. melting points, boiling points, spectral properties, and reactivities. Mixtures of diastereomers may be separated by crystallization or with high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McRaw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate.

The term “tautomer” or “tautomeric form” refers to structural isomers of different energies, which are interconvertible via a low energy barrier. For example, proton tautomers include interconversions via migration of a proton, such as keto-enol and iminenamine isomerizations.

The phrase “pharmaceutically acceptable salt” as used herein, refers to pharmaceutically acceptable organic or inorganic salts of a compound of the invention. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluene-sulfonate, and pamoate salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine, an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminium and lithium.

The phrase “pharmaceutically acceptable” indicates that the substance or composition must be compatible chemically and/or toxicologically, with the other ingredients comprising a formulation, and/or the mammal being treated therewith.

A “solvate” refers to an association or complex of one or more solvent molecules with a compound of the invention. Examples of solvents that form solvates include, but are not limited to, water, isopropanol, ethanol, methanol, DMSO, ethyl acetate, acetic acid, and ethanolamine. The term “hydrate” refers to the complex wherein the solvent molecule is water.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyl-oxycarbonyl, and 9-fluorenylmethylenoxycarbonyl (Fmoc). For a general description of protecting groups and their use, see T. W. Greene, Protective Groups in Organic Synthesis, John Wiley & Sons, New York, 1991.

The term “mammal” includes, but is not limited to, humans, mice, rats, guinea, pigs, monkeys, dogs, cats, horses, cows, pigs, and sheep.

The present invention provides triazine and pyrimidine compounds, and pharmaceutical formulations thereof, which are useful as therapeutic agents and novel diagnostic probes. Moreover, these compounds are potentially useful in the treatment of diseases, conditions and/or disorders modulated by protein kinases and lipid kinases.

More specifically, the invention relates to compounds of formula (I)

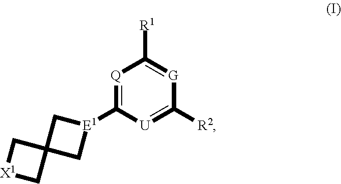

wherein

G is CH or N, Q is CH or N, and U is CH or N, with the proviso that at least two of G, Q and U are N, or one of G and U together with R² forms an anullated pyridine ring further substituted by R³, and the other one of G and U is N and Q is N;

E¹ and E² are, independently of each other, CR⁴ or N;

X¹ and X² are, independently of each other, CHR⁴, CH₂CH₂, NR⁴, NR⁴→O, or O;

R¹ is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, a linker carrying a reactive group and/or a tag, or

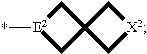

R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;

R³ is optionally substituted amino, optionally substituted C₆-C₂₀-aryl, or optionally substituted C₁-C₁₉-heteroaryl;

R⁴ is hydrogen, C₁-C₆-alkyl, C₁-C₆-acyl, C₁-C₆-acylamino-C₁-C₆-alkyl, a reactive group or a linker carrying a reactive group and/or a tag;

and tautomers, prodrugs, metabolites, solvates and pharmaceutically acceptable salts thereof.

If in formula (I) one of G and U together with R² forms an anullated pyridine ring further substituted by R³, the resulting compound has preferably the following structure (II) or (III):

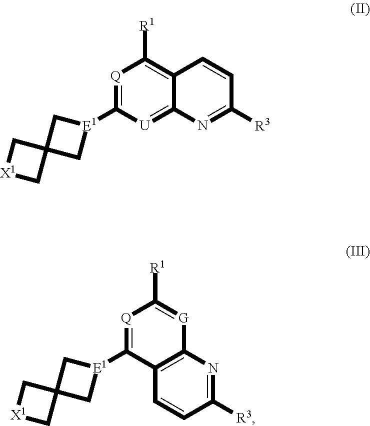

however, the substituent R³ may be located in meta or para position in relation to the anullated pyridine nitrogen atom, and not in the preferred ortho position as shown in formula (II) and (III).

In R¹ and R² with the meaning optionally substituted C₃-C₁₂-carbocyclyl, substituents considered are one or more groups halogen, C₁-C₆-alkyl, e.g. methyl or ethyl, halo-C₁-C₆-alkyl, e.g. difluoromethyl or trifluoromethyl, hydroxy-C₁-C₆-alkyl, e.g. hydroxy-methyl, C₁-C₆-alkoxy-C₁-C₆-alkyl, e.g. methoxyethyl, oxo-C₁-C₆-alkyl, e.g. formyl or 3-oxobutyl, carboxy-C₁-C₆-alkyl, e.g. carboxymethyl, C₁-C₆-alkoxycarbonyl-C₁-C₆-alkyl, e.g. methoxy- or ethoxycarbonylmethyl, optionally C₁-C₆-alkylated aminocarbonyl-C₁-C₆-alkyl, e.g. aminocarbonylmethyl or dimethylaminocarbonylmethyl, optionally C₁-C₆-alkylated or C₁-C₆-acylated amino-C₁-C₆-alkyl, e.g. aminomethyl, aminoethyl, dimethylaminoethyl, hydroxyethylaminoethyl, di(hydroxyethyl)aminoethyl, acetylaminomethyl, or acryloylamino-methyl; phenyl-C₁-C₆-alkyl, e.g. benzyl or phenethyl, C₂-C₆-alkenyl, e.g. vinyl or allyl, C₂-C₆-alkynyl, e.g. acetylenyl, hydroxy, C₁-C₆-alkoxy, e.g. methoxy or ethoxy, C₁-C₆-alkoxy-C₁-C₆-alkoxy, e.g. methoxyethoxy, oxo, optionally C₁-C₆-alkylated or C₁-C₂₀-acylated amino, e.g. amino, dimethylamino, hydroxyethylamino, di(hydroxyethyl)amino, acetylamino, acryloylamino, methacryloylamino, 2,3-epoxypropionylamino, fluoro-, chloro- or bromo-acetylamino, methoxycarbonylamino, methylaminocarbonylamino, pyridin-3-ylcarbonylamino, 2-aminopyridin-5-ylcarbonylamino, 2-amino-4-trifluoromethylpyridin-5-ylcarbonylamino, 2-aminopyridin-5-ylaminocarbonylamino, 2-aminopyrimidin-5-ylcarbonyl-amino, trifluoromethylsulfonylamino, or chloro- or bromomethylsulfonylamino; cyano, carboxy, C₁-C₆-alkoxycarbonyl, e.g. methoxycarbonyl, aminocarbonyl, or phenyl optionally carrying hydroxy or C₁-C₆-alkoxy groups, e.g. phenyl, hydroxyphenyl, di- or trihydroxy-phenyl, or hydroxydimethoxyphenyl.

In R¹, R² and R³ with the meaning optionally substituted C₆-C₂₀-aryl, substituents considered are the ones listed above as substituents for optionally substituted C₃-C₁₂-carbocyclyl (excluding oxo), and further one or more groups nitro, C₃-C₁₂-carbocyclyl, C₂-C₆-heterocyclyl optionally carrying one or more C₁-C₆-alkyl substituents, C₁-C₁₆-heteroaryl optionally carrying one or more C₁-C₆-alkyl, amino, C₁-C₆-alkylated amino or C₁-C₆-acylated amino substituents, C₁-C₆-alkylsulfonyl, e.g. methylsulfonyl or ethyl-sulfonyl, halo-C₁-C₆-alkylsulfonyl, e.g. trifluoromethylsulfonyl, optionally alkylated amino-sulfonyl, e.g. aminosulfonyl, methylaminosulfonyl, dimethylaminosulfonyl, hydroxyethyl-aminosulfonyl, or phenylsulfonyl.

In R¹ and R² with the meaning optionally substituted C₂-C₁₉-heterocyclyl, substituents considered are the ones listed above as substituents for optionally substituted C₃-C₁₂-carbocyclyl.

In R¹, R² and R³ with the meaning optionally substituted C₁-C₁₉-heteroaryl, substituents considered are the ones listed above as substituents for optionally substituted C₆-C₂₀-aryl.

In R¹ and R² with the meaning optionally substituted C₆-C₂₀-arylsulfonyl, substituents considered are the ones listed above as substituents for optionally substituted C₆-C₂₀-aryl.

In R¹ and R² with the meaning optionally substituted aminosulfonyl, substituents considered are are one or two groups C₁-C₆-alkyl, e.g. methyl or ethyl, hydroxy-C₁-C₆-alkyl, e.g. hydroxyethyl, C₁-C₆-alkoxy-C₁-C₆-alkyl, e.g. methoxyethyl, oxo-C₁-C₆-alkyl, e.g. 3-oxobutyl, optionally alkylated or acylated amino-C₁-C₆-alkyl, e.g. aminoethyl, dimethyl-aminoethyl, hydroxyethylaminoethyl, di(hydroxyethyl)aminoethyl, or acetylaminoethyl, phenyl-C₁-C₆-alkyl, e.g. benzyl or phenethyl, C₂-C₆-alkenyl, e.g. allyl, one group phenyl, or a ring-forming bifunctional substituent giving rise to optionally alkylated heterocyclyl-sulfonyl, e.g. pyrrolidinosulfonyl, piperidinosulfonyl, piperazinosulfonyl, methylpiperazino-sulfonyl, or morpholinosulfonyl.

In R³ with the meaning optionally substituted amino, substituents considered are one or two groups C₁-C₆-alkyl, e.g. methyl or ethyl, hydroxy-C₁-C₆-alkyl, e.g. hydroxyethyl or 2,3-dihydroxypropyl, C₁-C₆-alkoxy-C₁-C₆-alkyl, e.g. methoxyethyl, ethoxyethyl or 2,3-dimethoxypropyl, C₁-C₆-alkoxy-C₁-C₆-alkoxy-C₁-C₆-alkyl, e.g. ethoxyethoxyethyl, oxo-C₁-C₆-alkyl, e.g. 3-oxobutyl, optionally alkylated or acylated amino-C₁-C₆-alkyl, e.g. amino-ethyl, dimethylaminoethyl, hydroxyethylaminoethyl, di(hydroxyethyl)aminoethyl, or acetyl-aminoethyl, phenyl-C₁-C₆-alkyl, e.g. benzyl or phenethyl, C₂-C₆-alkenyl, e.g. allyl, one group phenyl, one group C₁-C₁₉-heteroaryl, e.g. 2-, 3- or 4-pyridyl, 2- or 4-pyrimidinyl, or 2- or 3-pyrrolyl, or a ring-forming bifunctional substituent giving rise to optionally alkylated heterocyclyl, e.g. pyrrolidino, piperidino, piperazino, methylpiperazino, morpholino or dimethylmorpholino.

Preferably, G, Q and U are N, or one of G and U together with R² forms an anullated pyridine ring further substituted by R³ of formula (II) or formula (III), and the other one of G and U is N and Q is N. Most preferred, G, Q and U are N.

Preferably, E¹ and E² are N.

Preferably, X¹ and X² are, independently of each other, CH₂, CH₂CH₂, NR⁴, NR⁴→O, or O; more preferably NR⁴ or O, most preferably O;

Preferably, R¹ is optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, or

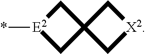

More preferably R¹ is optionally substituted

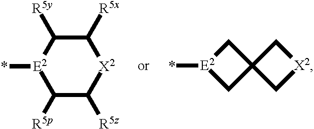

wherein R^{5x}, R^{5y}, R^{5z} and R^{5p} are, independently of each other, hydrogen, halogen, cyano, optionally substituted C₁-C₆ alkyl, C₂-C₆ alkenyl, or C₂-C₆ alkynyl, or one or two of R^{5x}, R^{5y}, R^{5z} and R^{5p} are two geminal substituents methyl and the other ones are hydrogen, or R^{5x} and R^{5y}, or R^{5z} and R^{5p} form together an anullated five- or six-membered carbocyclyl, heterocyclyl, aryl or heteroaryl ring, or R^{5x} and R^{5p} form together bridging ethylene, or R^{5y} and R^{5p} form together bridging ethylene, and E² and X² have the indicated meanings.

Most preferably R¹ is (S)-2-methylmorpholino; (R)-2-methylmorpholino; 2-(amino-carbonylmethyl)morpholino; 2-(benzamidomethyl)morpholino; (2R,6S)-2,6-dimethyl-morpholino; (2R,6R)-2,6-dimethylmorpholino; (R)-3-methylmorpholino; (S)-3-methyl-morpholino; (2R,3R)-2,3-dimethylmorpholino; (2S,5S)-2,5-dimethylmorpholino; (3S,5R)-3,5-dimethylmorpholino; (3S,5S)-3,5-dimethylmorpholino; octahydrocyclopenta[b][1,4]-oxazin-4-yl; octahydro-2H-benzo[b][1,4]oxazin-4-yl; 3,4-dihydro-2H-benzo[b][1,4]oxazin-4-yl; 3-methoxycarbonylmethyl-2-methylmorpholino; 2-(methoxycarbonylmethyl)morpholino; 3-(methoxycarbonylmethyl)morpholino; 2-vinylmorpholino; 2-(methoxycarbonylmethyl)-5-methylmorpholino; 3-(aminomethyl)morpholino; 2-(aminomethyl)morpholino; 2-cyano-morpholino; 2-(carboxymethyl)morpholino; 3-(hydroxymethyl)morpholino; 2-(hydroxy-methyl)morpholino; 2-(acetamidomethyl)morpholino; 2-(pyrrolidinocarbonylmethyl)-morpholino; 2-(aminocarbonyl)morpholino; 3-(aminocarbonyl)morpholino; 3-cyano-morpholino; 2,2,6,6-tetramethylmorpholino; 2,2,6-trimethylmorpholino; 8-oxa-3-aza-bicyclo[3.2.1]octan-3-yl; (1S,5R)-8-oxa-3-azabicyclo[3.2.1]octan-3-yl; or (1R,5S)-3-oxa-8-azabicyclo[3.2.1]octan-8-yl.

Likewise preferred are compounds wherein R¹ is piperidino, piperazino, 4-methyl-piperazino; 4-(methoxycarbonyl)piperazino, or 4-(methylsulfonyl)piperazino.

Even more preferred are compounds wherein R¹ is (S)-2-methylmorpholino; (R)-2-methylmorpholino; (2R,6S)-2,6-dimethylmorpholino; (2R,6R)-2,6-dimethylmorpholino; (R)-3-methylmorpholino; (S)-3-methylmorpholino; (2R,3R)-2,3-dimethylmorpholino; (2S,5S)-2,5-dimethylmorpholino; (3S,5R)-3,5-dimethylmorpholino; (3S,5S)-3,5-dimethyl-morpholino; octahydrocyclopenta[b][1,4]oxazin-4-yl; 2,2,6,6-tetramethylmorpholino; 2,2,6-trimethylmorpholino; 8-oxa-3-azabicyclo[3.2.1]octan-3-yl; (1S,5R)-8-oxa-3-azabicyclo-[3.2.1]octan-3-yl; or (1R,5S)-3-oxa-8-azabicyclo[3.2.1]octan-8-yl.

Likewise preferred are compounds wherein R¹ is 4-methylpiperazino; 4-(methoxy-carbonyl)piperazino, or 4-(methylsulfonyl)piperazino.

Likewise preferred are compounds wherein R¹ is

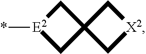

### and E² is N and X² is O.

Preferably, R² is optionally substituted C₆-C₂₀ aryl or optionally substituted C₁-C₂₀ heteroaryl. In preferred R², optionally substituted C₆-C₂₀ aryl is preferably optionally substituted phenyl. Substituents considered for phenyl are those listed above for C₆-C₂₀ aryl, preferably one or more groups halogen, C₁-C₆-alkyl, halo-C₁-C₆-alkyl, hydroxy, C₁-C₆-alkoxy, and optionally C₁-C₆-alkylated or C₁-C₂₀-acylated amino.

In preferred R², optionally substituted C₁-C₂₀ heteroaryl is preferably optionally substituted pyridinyl, imidazolyl, pyrimidinyl, pyrazolyl, triazolyl, pyrazinyl, tetrazolyl, furyl, thienyl, oxazolyl, isoxazolyl, thiazolyl, isothiazolyl, pyrrolyl, indolyl, benzimidazolyl, indazolyl, oxadiazolyl, or thiadiazolyl. Substituents considered for the mentioned preferred heteroaryl are those listed above for C₁-C₂₀ heteroaryl, preferably one or more groups halogen, C₁-C₆-alkyl, halo-C₁-C₆-alkyl, hydroxy, C₁-C₆-alkoxy, optionally C₁-C₆-alkylated or C₁-C₂₀-acylated amino, pyrdiyl, aminopyridyl, or optionally substituted phenyl, preferably phenyl or phenyl carrying one or more hydroxy and/or C₁-C₆-alkoxy groups.

More preferably R² is meta- or para-substituted phenyl or 2,4-, 3,4- or 3,5-disubstituted phenyl, wherein the substituents are selected from halogen, C₁-C₆-alkyl, halo-C₁-C₆-alkyl, hydroxy, C₁-C₆-alkoxy, optionally C₁-C₆-alkylated or C₁-C₂₀-acylated amino. Even more preferred R² is meta- or para-substituted phenyl, wherein the substituent is hydroxy, C₁-C₆-alkoxy, amino, C₁-C₆-alkylamino, di(C₁-C₆-alkyl)amino, or C₁-C₈-acylamino, wherein C₁-C₈-acyl is a C₁-C₇-alkyl, C₂-C₇alkenyl, C₂-C₇-alkynyl, C₁-C₇-carbocyclyl, phenyl, C₂-C₆-heterocyclyl, or C₁-C₅-heteroaryl group connected to carbonyl, sulfonyl, oxycarbonyl or aminocarbonyl.

Likewise more preferably R² is optionally substituted pyridinyl, imidazolyl, pyrimidinyl, furyl, indolyl, benzimidazolyl, indazolyl, oxadiazolyl, or thiadiazolyl, wherein the substituents are selected from halogen, C₁-C₆-alkyl, halo-C₁-C₆-alkyl, optionally C₁-C₆-alkylated or C₁-C₂₀-acylated amino, phenyl carrying one or more hydroxy and/or C₁-C₆-alkoxy groups, pyridyl, aminopyridyl, and combinations thereof. Even more preferred R² is optionally substituted pyridinyl, imidazolyl, pyrimidinyl, furyl, indolyl, benzimidazolyl, indazolyl, oxadiazolyl, or thiadiazolyl, wherein the substituents are selected from C₁-C₆-alkyl, halo-C₁-C₆-alkyl, dimethoxyhydroxyphenyl, pyridyl, aminopyridyl, amino or C₁-C₈-acylamino, wherein C₁-C₈-acyl is a C₁-C₇-alkyl, halo-C₁-C₇alkyl, epoxy-C₁-C₇alkyl, C₂-C₇-alkenyl, pyridyl or aminopyridyl group connected to carbonyl, oxycarbonyl or amino-carbonyl; and combinations thereof.

In particular R² is meta- or para-substituted phenyl, wherein the substituent is hydroxy or C₁-C₈-acylamino, wherein C₁-C₈-acyl is a C₁-C₇-alkyl, C₂-C₇alkenyl, pyridyl, aminopyridyl, amino-trifluormethyl-pyridyl, pyrimidinyl or aminopyridmidinyl group connected to carbonyl, oxycarbonyl or aminocarbonyl; or optionally substituted pyridinyl, imidazolyl, pyrimidinyl, furyl, indolyl, benzimidazolyl, indazolyl, wherein the substituents are selected from methyl, difloromethyl, trifluoromethyl, dimethoxyhydroxyphenyl, pyridyl, aminopyridyl, amino, haloacetylamino, acryloylamino, methacryloylamino, ethylamino-carbonylamino, ethoxycarbonylamino, pyridyl, and combinations thereof.

Preferably R³ is C₁-C₆-alkylamino, di-C₁-C₆-alkylamino, hydroxy-C₁-C₆-alkylamino, di(hydroxy-C₁-C₆-alkyl)amino, C₁-C₆-alkoxy-C₁-C₆-alkylamino, di(C₁-C₆-alkoxy-C₁-C₆-alkyl)amino, C₁-C₆-alkoxy-C₁-C₆-alkoxy-C₁-C₆-alkylamino, oxo-C₁-C₆-alkylamino, amino-C₁-C₆-alkylamino, C₁-C₆-alkylamino-C₁-C₆-alkylamino, di(C₁-C₆-alkyl)amino-C₁-C₆-alkyl-amino, hydroxy-C₁-C₆-alkylamino-C₁-C₆-alkylamino, di(hydroxy-C₁-C₆-alkyl)amino-C₁-C₆-alkylamino, C₁-C₆-alkylcarbonylamino-C₁-C₆-alkylamino, phenyl-C₁-C₆-alkylamino, C₂-C₆-alkenylamino, phenylamino, pyridylamino, pyrimidinylamino, pyrrolylamino, pyrrolidino, piperidino, piperazino, 4-methylpiperazino, morpholino or dimethylmorpholino.

Likewise preferably, R³ is phenyl or naphthyl, optionally substituted by one or more groups halogen, C₁-C₆-alkyl, halo-C₁-C₆-alkyl, hydroxy-C₁-C₆-alkyl, C₁-C₆-alkoxy-C₁-C₆-alkyl, oxo-C₁-C₆-alkyl, carboxy-C₁-C₆-alkyl, C₁-C₆-alkoxycarbonyl-C₁-C₆-alkyl, amino-carbonyl-C₁-C₆-alkyl, C₁-C₆-alkylaminocarbonyl-C₁-C₆-alkyl, amino-C₁-C₆-alkyl, C₁-C₆-alkylamino-C₁-C₆-alkyl, C₁-C₆-alkylcarbonylamino-C₁-C₆-alkyl, C₂-C₆-alkenylcarbonyl-amino-C₁-C₆-alkyl, phenyl-C₁-C₆-alkyl, C₂-C₆-alkenyl, C₂-C₆-alkynyl, hydroxy, C₁-C₆-alkoxy, C₁-C₆-alkoxy-C₁-C₆-alkoxy, amino, C₁-C₆-alkylamino, di-C₁-C₆-alkylamino, hydroxy-C₁-C₆-alkylamino, di(hydroxy-C₁-C₆-alkyl)amino, C₁-C₆-alkylcarbonylamino, halo-C₁-C₆-alkylcarbonylamino, C₂-C₆-alkenylcarbonylamino, C₁-C₆-alkyloxycarbonylamino, C₁-C₆-alkylaminocarbonylamino, pyridinylcarbonylamino, aminopyridinylcarbonylamino, amino-trifluoromethyl-pyridinylcarbonylamino, halo-C₁-C₆-alkylsulfonylamino, cyano, carboxy, C₁-C₆-alkoxycarbonyl, or aminocarbonyl.

Likewise preferably, R³ is optionally substituted pyridinyl, imidazolyl, pyrimidinyl, furyl, indolyl, benzimidazolyl, or indazolyl, wherein the substituents are selected from C₁-C₆-alkyl, halo-C₁-C₆-alkyl, amino or C₁-C₈-acylamino, wherein C₁-C₈-acyl is a C₁-C₇-alkyl, halo-C₁-C₇alkyl, epoxy-C₁-C₇alkyl, C₂-C₇alkenyl, pyridyl or aminopyridyl group connected to carbonyl, oxycarbonyl or aminocarbonyl; and combinations thereof.

More preferably, R³ is C₁-C₆-alkylamino, di-C₁-C₆-alkylamino, hydroxy-C₁-C₆-alkylamino, di(hydroxy-C₁-C₆-alkyl)amino, C₁-C₆-alkoxy-C₁-C₆-alkylamino, di(C₁-C₆-alkoxy-C₁-C₆-alkyl)amino, amino-C₁-C₆-alkylamino, C₁-C₆-alkylamino-C₁-C₆-alkylamino, di(C₁-C₆-alkyl)amino-C₁-C₆-alkylamino, C₁-C₆-alkylcarbonylamino-C₁-C₆-alkylamino, C₂-C₆-alkenylamino, pyridylamino, pyrimidinylamino, morpholino; phenyl, optionally substituted by one or more groups halogen, C₁-C₆-alkyl, halo-C₁-C₆-alkyl, hydroxy-C₁-C₆-alkyl, C₁-C₆-alkoxy-C₁-C₆-alkyl, hydroxy, C₁-C₆-alkoxy, C₁-C₆-alkoxy-C₁-C₆-alkoxy, amino, C₁-C₆-alkylamino, di-C₁-C₆-alkylamino, hydroxy-C₁-C₆-alkylamino, di(hydroxy-C₁-C₆-alkyl)amino, C₁-C₆-alkylcarbonylamino, halo-C₁-C₆-alkylcarbonylamino, C₂-C₆-alkenylcarbonylamino; pyridinyl or pyrimidinyl, optionally substituted by one or more groups C₁-C₆-alkyl, halo-C₁-C₆-alkyl, amino or C₁-C₈-acylamino, wherein C₁-C₈-acyl is a C₁-C₇-alkyl, halo-C₁-C₇-alkyl, epoxy-C₁-C₇-alkyl, or C₂-C₇-alkenyl, connected to carbonyl, oxycarbonyl or aminocarbonyl.

In particular, R³ is phenyl, hydroxy-phenyl, methoxy-phenyl, hydroxy-dimethoxy-phenyl, hydroxymethyl-phenyl, hydroxymethyl-methoxy-phenyl, hydroxymethyl-dimethoxy-phenyl, pyridinyl, furanyl, or thienyl.

Preferably R⁴ is hydrogen, methyl, a reactive group selected from acryloyl, methacryloyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-tri-chloroacetyl, methylsulfonyloxyacetyl, 2-chloropropionyl, 2,3-epoxypropionyl, (phenylthio)-thiocarbonyl, 2-nitrophenoxycarbonyl, 4-fluorophenoxycarbonyl, and 4-(3-(trifluoromethyl)-3H-diazirin-3-yl)benzamide, a chain of 1 to 20 optionally substituted methylene groups either directly linked to X¹ or X², or linked to the reactive group, or such chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally a reactive group selected from acryloyl, methacryloyl, 4-amino-but-2-enoyl, 4-dimethyl-amino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-trichloroacetyl, methylsulfonyloxyacetyl, 2-chloropropionyl, 2,3-epoxypropionyl, (phenylthio)thiocarbonyl, 2-nitrophenoxycarbonyl, and 4-fluorophenoxycarbonyl.

More preferably R⁴ is hydrogen, methyl, a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, and 3-(dimethylamino)-1-propene-1-sulfonyl, a chain of 1 to 20 methylene groups either directly linked to X¹ or X², or linked to the reactive group, such chain that is substituted by oxo, C₁-C₆ alkyl, a further chain of 1 to 6 methylene groups, phenyl, phenylene, or residues of naturally occurring amino acids, or such optionally substituted chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally one further reactive group selected from acryloyl, methacryloyl, 4-dimethylamino-but-2-enoyl, and 4-(dimethylamino)-2,3-epoxy-butanoyl.

In particular R⁴ is hydrogen, methyl, a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, and 4-(3-(trifluoromethyl)-3H-diazirin-3-yl)benzamide, a chain of 1 to 20 methylene groups substituted by residues of naturally occurring amino acids, wherein one or more methylene groups are replaced by a carboxamide group, carrying a naturally occurring amino acid, 4-amino-but-2-enoyl or 3-amino-1-propene-1-sulfonyl, acylated at the amino group with a chain of 2 to 6 methylene groups substituted by oxo and carrying biotin or a fluorophore, or 4-(3-(trifluoromethyl)-3H-diazirin-3-yl)benzamide, substituted at position 2 with a chain of 1 to 20 methylene groups, wherein one or more methylene groups are replaced by a carboxamide group and by oxygen, carrying biotin.

Preferred are compounds of formula (I)

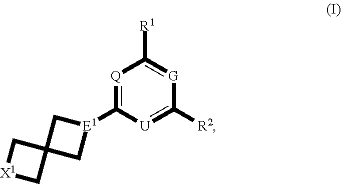

wherein

G is CH or N, Q is CH or N, and U is CH or N, with the proviso that at least two of G, Q and U are N;

E¹ and E² are, independently of each other, CR⁴ or N;

X¹ and X² are, independently of each other, CHR⁴, CH₂CH₂, NR⁴, NR⁴→O, or O;

R¹ has one of the preferred, more preferred, most preferred, or even more preferred meanings given above;

R² has one of the preferred, more preferred or particular meanings given above; and

R⁴ has one of the preferred, more preferred or particular meanings given above;

and tautomers, solvates and pharmaceutically acceptable salts thereof.

Also preferred are compounds of formula (I), wherein

G is CH or N, Q is CH or N, and U is CH or N, with the proviso that at least two of G, Q and U are N;

E¹ and E² are N;

X¹ and X² are, independently of each other, NR⁴ or O;

R¹ has one of the preferred, more preferred, most preferred, or even more preferred meanings given above;

R² has one of the preferred, more preferred or particular meanings given above; and

R⁴ has one of the preferred, more preferred or particular meanings given above;

and tautomers, solvates and pharmaceutically acceptable salts thereof.

Also preferred are compounds of formula (II) or (III):

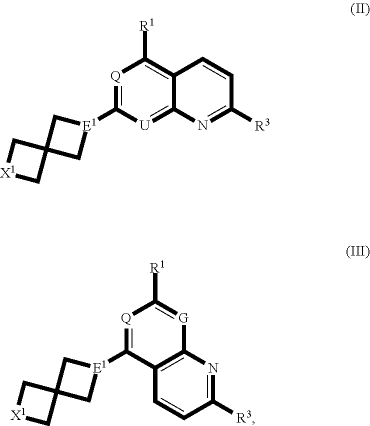

wherein

E¹ and E² are N;

X¹ and X² are, independently of each other, NR⁴ or O;

R¹ has one of the preferred, more preferred, most preferred, or even more preferred meanings given above;

R³ has one of the preferred, more preferred or particular meanings given above; and

R⁴ has one of the preferred, more preferred or particular meanings given above;

and tautomers, solvates and pharmaceutically acceptable salts thereof.

Particularly preferred are compounds of formula (II) or (III), wherein E¹ and E² are N; and X¹ and X² are O.

Also preferred are compounds of the formula

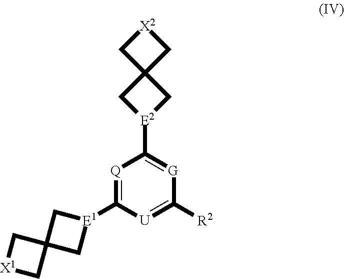

wherein

G is CH or N, Q is CH or N, and U is CH or N, with the proviso that at least two of G, Q and U are N, or one of G and U together with R² forms an anullated pyridine ring further substituted by R³, and the other one of G and U is N and Q is N;

E¹ and E² are, independently of each other, CR⁴ or N;

X¹ and X² are, independently of each other, CHR⁴, CH₂CH₂, NR⁴, NR⁴→O, or O;

R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;

R³ is optionally substituted amino, optionally substituted C₆-C₂₀-aryl, or optionally substituted C₁-C₁₉-heteroaryl;

R⁴ is hydrogen, C₁-C₆-alkyl, C₁-C₆-acyl, C₁-C₆-acylamino-C₁-C₆-alkyl, a reactive group or a linker carrying a reactive group and/or a tag;

and tautomers, prodrugs, metabolites, solvates and pharmaceutically acceptable salts thereof.

Preferred are compounds of formula (IV), wherein

G is CH or N, Q is CH or N, and U is CH or N, with the proviso that at least two of G, Q and U are N;

E¹ and E² are, independently of each other, CR⁴ or N;

X¹ and X² are, independently of each other, CHR⁴, CH₂CH₂, NR⁴, NR⁴→O, or O;

R² has one of the preferred, more preferred or particular meanings given above; and

R⁴ has one of the preferred, more preferred or particular meanings given above;

and tautomers, solvates and pharmaceutically acceptable salts thereof.

More preferred are compounds of formula (IV), wherein

E¹ and E² are N; and

X¹ and X² are, independently of each other, NR⁴ or O; preferably O.

Also preferred are compounds of formula

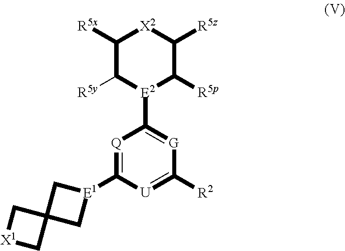

wherein

G is CH or N, Q is CH or N, and U is CH or N, with the proviso that at least two of G, Q and U are N, or one of G and U together with R² forms an anullated pyridine ring further substituted by R³, and the other one of G and U is N and Q is N;

E¹ and E² are, independently of each other, CR⁴ or N;

X¹ and X² are, independently of each other, CHR⁴, CH₂CH₂, NR⁴, NR⁴→O, or O;

R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;

R³ is optionally substituted amino, optionally substituted C₆-C₂₀-aryl, or optionally substituted C₁-C₁₉-heteroaryl;

R⁴ is hydrogen, C₁-C₆-alkyl, C₁-C₆-acyl, C₁-C₆-acylamino-C₁-C₆-alkyl, a reactive group or a linker carrying a reactive group and/or a tag;

R^{5x}, R^{5y}, R^{5z} and R^{5p} are, independently of each other, hydrogen, halogen, cyano, optionally substituted C₁-C₆ alkyl, C₂-C₆ alkenyl, or C₂-C₆ alkynyl, or one or two of R^{5x}, R^{5y}, R^{5z} and R^{5p} are two geminal substituents methyl and the other ones are hydrogen, or R^{5x} and R^{5y}, or R^{5z} and R^{5p} form together an anullated five- or six-membered carbocyclyl, heterocyclyl, aryl or heteroaryl ring, or R^{5x} and R^{5p} form together bridging ethylene, or R^{5y} and R^{5p} form together bridging ethylene;

and tautomers, prodrugs, metabolites, solvates and pharmaceutically acceptable salts thereof.

Preferred are compounds of formula (V), wherein

G is CH or N, Q is CH or N, and U is CH or N, with the proviso that at least two of G, Q and U are N;

E¹ and E² are, independently of each other, CR⁴ or N;

X¹ and X² are, independently of each other, CHR⁴, CH₂CH₂, NR⁴, NR⁴→O, or O;

R² has one of the preferred, more preferred or particular meanings given above;

R⁴ has one of the preferred, more preferred or particular meanings given above; and

R^{5x}, R^{5y}, R^{5z} and R^{5p} have the meanings indicated;

and tautomers, solvates and pharmaceutically acceptable salts thereof.

More preferred are compounds of formula (V), wherein

E¹ and E² are N; and

X¹ and X² are, independently of each other, NR⁴ or O; preferably O.

Most preferred are the compounds of the examples, of Table 1, of Table 2, of Table 3, and particularly of Table 4 below.

Among these, preferred compounds are selected from the group consisting of 6-amino-N-(3-(4-morpholino-6-(2-oxa-6-azaspiro[3.3]heptan-6-yl)-1,3,5-triazin-2-yl)phenyl)-nicotinamide (example 111); N-(3-(4-morpholino-6-(2-oxa-6-azaspiro[3.3]heptan-6-yl)-1,3,5-triazin-2-yl)phenyl)nicotinamide (125); methyl (4-(4-morpholino-6-(2-oxa-6-azaspiro-[3.3]heptan-6-yl)-1,3,5-triazin-2-yl)phenyl)carbamate (131); methyl (4-(4-(8-oxa-3-aza-bicyclo[3.2.1]octan-3-yl)-6-(2-oxa-6-azaspiro[3.3]heptan-6-yl)-1,3,5-triazin-2-yl)phenyl)-carbamate (132); 1-methyl-3-(4-(4-morpholino-6-(2-oxa-6-azaspiro[3.3]heptan-6-yl)-1,3,5-triazin-2-yl)phenyl)urea (141); 1-(4-(4-(8-oxa-3-azabicyclo[3.2.1]octan-3-yl)-6-(2-oxa-6-azaspiro[3.3]heptan-6-yl)-1,3,5-triazin-2-yl)phenyl)-3-methylurea (146); 1-ethyl-3-(4-(4-morpholino-6-(2-oxa-6-azaspiro[3.3]heptan-6-yl)-1,3,5-triazin-2-yl)phenyl)urea (151); 1-(4-(4-(8-oxa-3-azabicyclo[3.2.1]octan-3-yl)-6-(2-oxa-6-azaspiro[3.3]heptan-6-yl)-1,3,5-triazin-2-yl)phenyl)-3-ethylurea (155); 1-ethyl-3-(5-(4-morpholino-6-(2-oxa-6-azaspiro[3.3]heptan-6-yl)-1,3,5-triazin-2-yl)pyridin-2-yl)urea (160); 1-(5-(4-(8-oxa-3-azabicyclo[3.2.1]octan-3-yl)-6-(2-oxa-6-azaspiro[3.3]heptan-6-yl)-1,3,5-triazin-2-yl)pyridin-2-yl)-3-ethylurea (164); 1-ethyl-3-(5-(4-morpholino-6-(2-oxa-6-azaspiro[3.3]heptan-6-yl)-1,3,5-triazin-2-yl)pyrimidin-2-yl)urea (169); 1-(5-(4-(8-oxa-3-azabicyclo[3.2.1]octan-3-yl)-6-(2-oxa-6-azaspiro[3.3]-heptan-6-yl)-1,3,5-triazin-2-yl)pyrimidin-2-yl)-3-ethylurea (173); 1-(4-(4-(dimethylamino)-piperidine-1-carbonyl)phenyl)-3-(4-(4-morpholino-6-(2-oxa-6-azaspiro[3.3]heptan-6-yl)-1,3,5-triazin-2-yl)phenyl)urea (196); 1-(4-(4-(dimethylamino)piperidine-1-carbonyl)phenyl)-3-(5-(4-morpholino-6-(2-oxa-6-azaspiro[3.3]heptan-6-yl)-1,3,5-triazin-2-yl)pyridin-2-yl)urea (200); 1-(4-(4-(dimethylamino)piperidine-1-carbonyl)phenyl)-3-(5-(4-morpholino-6-(2-oxa-6-azaspiro[3.3]heptan-6-yl)-1,3,5-triazin-2-yl)pyrimidin-2-yl)urea (204); 5-(4-morpholino-6-(2-oxa-6-azaspiro[3.3]heptan-6-yl)-1,3,5-triazin-2-yl)pyridin-2-amine (215); 4-methyl-5-(4-morpholino-6-(2-oxa-6-azaspiro[3.3]heptan-6-yl)-1,3,5-triazin-2-yl)pyridin-2-amine (220); 5-(4-(8-oxa-3-azabicyclo[3.2.1]octan-3-yl)-6-(2-oxa-6-azaspiro[3.3]heptan-6-yl)-1,3,5-triazin-2-yl)-4-methylpyridin-2-amine (221); 5-(4-morpholino-6-(2-oxa-6-azaspiro[3.3]-heptan-6-yl)-1,3,5-triazin-2-yl)-4-(trifluoromethyl)pyridin-2-amine (225); 5-(4-morpholino-6-(2-oxa-6-azaspiro[3.3]heptan-6-yl)pyrimidin-2-yl)-4-(trifluoromethyl)pyridin-2-amine (227); 5-(6-morpholino-2-(2-oxa-6-azaspiro[3.3]heptan-6-yl)pyrimidin-4-yl)-4-(trifluoromethyl)-pyridin-2-amine (228); 5-(2-morpholino-6-(2-oxa-6-azaspiro[3.3]heptan-6-yl)pyrimidin-4-yl)-4-(trifluoromethyl)pyridin-2-amine (229); 5-(4-(6-methyl-2,6-diazaspiro[3.3]heptan-2-yl)-6-morpholino-1,3,5-triazin-2-yl)-4-(trifluoromethyl)pyridin-2-amine (231); 5-(4-morpholino-6-(2-oxa-6-azaspiro[3.3]heptan-6-yl)-1,3,5-triazin-2-yl)pyrimidin-2-amine (251); 5-(4-(8-oxa-3-azabicyclo[3.2.1]octan-3-yl)-6-(2-oxa-6-azaspiro[3.3]heptan-6-yl)-1,3,5-triazin-2-yl)pyrimidin-2-amine (252); 4-morpholino-6-(2-oxa-6-azaspiro[3.3]heptan-6-yl)-[2,5′-bipyrimidin]-2′-amine (253); 6-morpholino-2-(2-oxa-6-azaspiro[3.3]heptan-6-yl)-[4,5′-bipyrimidin]-2′-amine (254); 2-morpholino-6-(2-oxa-6-azaspiro[3.3]heptan-6-yl)-[4,5′-bipyrimidin]-2′-amine (255); 5-(4-(6-methyl-2,6-diazaspiro[3.3]heptan-2-yl)-6-morpholino-1,3,5-triazin-2-yl)pyrimidin-2-amine (257); 1-(6-(4-(2-aminopyrimidin-5-yl)-6-morpholino-1,3,5-triazin-2-yl)-2,6-diazaspiro[3.3]heptan-2-yl)prop-2-en-1-one (259); 1-(6-(4-(2-amino-pyrimidin-5-yl)-6-morpholino-1,3,5-triazin-2-yl)-2,6-diazaspiro[3.3]heptan-2-yl)-2-chloro-ethanone (261); 4-methyl-5-(4-morpholino-6-(2-oxa-6-azaspiro[3.3]heptan-6-yl)-1,3,5-triazin-2-yl)pyrimidin-2-amine (268); 5-(4-(8-oxa-3-azabicyclo[3.2.1]octan-3-yl)-6-(2-oxa-6-azaspiro[3.3]heptan-6-yl)-1,3,5-triazin-2-yl)-4-methylpyrimidin-2-amine (269); 5-(4-morpholino-6-(2-oxa-6-azaspiro[3.3]heptan-6-yl)-1,3,5-triazin-2-yl)-4-(trifluoromethyl)-pyrimidin-2-amine (273); 4-morpholino-6-(2-oxa-6-azaspiro[3.3]heptan-6-yl)-4′-(trifluoro-methyl)-[2,5′-bipyrimidin]-2′-amine (276); 6-morpholino-2-(2-oxa-6-azaspiro[3.3]heptan-6-yl)-4′-(trifluoromethyl)-[4,5′-bipyrimidin]-2′-amine (277); 2-morpholino-6-(2-oxa-6-azaspiro-[3.3]heptan-6-yl)-4′-(trifluoromethyl)-[4,5′-bipyrimidin]-2′-amine (278); 6-(4-(2-(difluoro-methyl)-1H-benzo[d]imidazol-1-yl)-6-morpholino-1,3,5-triazin-2-yl)-2-oxa-6-azaspiro[3.3]-heptane (299); 6-(2-(2-(difluoromethyl)-1H-benzo[d]imidazol-1-yl)-6-morpholinopyrimidin-4-yl)-2-oxa-6-azaspiro[3.3]heptane (300); 6-(4-(2-(difluoromethyl)-1H-benzo[d]imidazol-1-yl)-6-morpholinopyrimidin-2-yl)-2-oxa-6-azaspiro[3.3]heptane (301); 6-(6-(2-(difluoro-methyl)-1H-benzo[d]imidazol-1-yl)-2-morpholinopyrimidin-4-yl)-2-oxa-6-azaspiro[3.3]-heptane (302); 4-(4-(2-(difluoromethyl)-1H-benzo[d]imidazol-1-yl)-6-(2,6-diazaspiro[3.3]-heptan-2-yl)-1,3,5-triazin-2-yl)morpholine (303); 4-(4-(2-(difluoromethyl)-1H-benzo[d]-imidazol-1-yl)-6-(6-methyl-2,6-diazaspiro[3.3]heptan-2-yl)-1,3,5-triazin-2-yl)morpholine (304); 3-(4-(2-(difluoromethyl)-1H-benzo[d]imidazol-1-yl)-6-(2-oxa-6-azaspiro[3.3]heptan-6-yl)-1,3,5-triazin-2-yl)-8-oxa-3-azabicyclo[3.2.1]octane (308); N-(2-(6-(4-(2-(difluoro-methyl)-1H-benzo[d]imidazol-1-yl)-6-morpholino-1,3,5-triazin-2-yl)-2,6-diazaspiro[3.3]-heptan-2-yl)ethyl)acrylamide (312); 2-chloro-N-(2-(6-(4-(2-(difluoromethyl)-1H-benzo[d]-imidazol-1-yl)-6-morpholino-1,3,5-triazin-2-yl)-2,6-diazaspiro[3.3]heptan-2-yl)ethyl)acetamide (316); 1-(6-(4-(2-(difluoromethyl)-1H-benzo[d]imidazol-1-yl)-6-morpholino-1,3,5-triazin-2-yl)-2,6-diazaspiro[3.3]heptan-2-yl)prop-2-en-1-one (317); 2-chloro-1-(6-(4-(2-(difluoromethyl)-1H-benzo[d]imidazol-1-yl)-6-morpholino-1,3,5-triazin-2-yl)-2,6-diazaspiro-[3.3]heptan-2-yl)ethanone (323); 2,6-dimethoxy-4-(1-(4-morpholino-6-(2-oxa-6-azaspiro-[3.3]heptan-6-yl)-1,3,5-triazin-2-yl)-1H-imidazol-4-yl)phenol (345); 4-(1-(4-(8-oxa-3-aza-bicyclo[3.2.1]octan-3-yl)-6-(2-oxa-6-azaspiro[3.3]heptan-6-yl)-1,3,5-triazin-2-yl)-1H-imidazol-4-yl)-2,6-dimethoxyphenol (346); 2,6-dimethoxy-4-(5-(4-morpholino-6-(2-oxa-6-azaspiro[3.3]heptan-6-yl)-1,3,5-triazin-2-yl)furan-2-yl)phenol (351); 4-(5-(4-(8-oxa-3-azabicyclo[3.2.1]octan-3-yl)-6-(2-oxa-6-azaspiro[3.3]heptan-6-yl)-1,3,5-triazin-2-yl)furan-2-yl)-2,6-dimethoxyphenol (352); (E)-3-((6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)prop-2-en-1-amine (366); (E)-3-((6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)-N,N-dimethylprop-2-en-1-amine (367); (E)-1-(6-(4-(2-(difluoromethyl)-1H-benzo[d]imidazol-1-yl)-6-morpholino-1,3,5-triazin-2-yl)-2,6-diazaspiro[3.3]heptan-2-yl)-4-(dimethylamino)but-2-en-1-one (368); N-((E)-3-((6-(4-(2-(difluoromethyl)-1H-benzo[d]imidazol-1-yl)-6-morpholino-1,3,5-triazin-2-yl)-2,6-diazaspiro-[3.3]heptan-2-yl)sulfonyl)allyl)-5-((3aS,4S,6aR)-2-oxohexahydro-1H-thieno[3,4-d]imidazol-4-yl)pentanamide (369); N-((E)-4-(6-(4-(2-(difluoromethyl)-1H-benzo[d]imidazol-1-yl)-6-morpholino-1,3,5-triazin-2-yl)-2,6-diazaspiro[3.3]heptan-2-yl)-4-oxobut-2-en-1-yl)-5-((3aS,4S,6aR)-2-oxohexahydro-1H-thieno[3,4-d]imidazol-4-yl)pentanamide (370); (E)-3-(4-(2-((6-(6-(4-(2-(difluoromethyl)-1H-benzo[d]imidazol-1-yl)-6-morpholino-1,3,5-triazin-2-yl)-2,6-diazaspiro[3.3]heptan-2-yl)-6-oxohexyl)amino)-2-oxoethoxy)styryl)-5,5-difluoro-7-(thiophen-2-yl)-5H-dipyrrolo[1,2-c:2′,1′-f][1,3,2]diazaborinin-4-ium-5-uide (371); (5-(4-(8-oxa-3-azabicyclo[3.2.1]octan-3-yl)-2-(2-oxa-6-azaspiro[3.3]heptan-6-yl)pyrido[2,3-d]-pyrimidin-7-yl)-2-methoxyphenyl)methanol (372); (5-(4-((3R,5S)-3,5-dimethylmorpholino)-2-(2-oxa-6-azaspiro[3.3]heptan-6-yl)pyrido[2,3-d]pyrimidin-7-yl)-2-methoxyphenyl)-methanol (374); and (2-methoxy-5-(4-morpholino-2-(2-oxa-6-azaspiro[3.3]heptan-6-yl)-pyrido[2,3-d]pyrimidin-7-yl)phenyl)methanol (375).

Particularly preferred are compounds selected from the group consisting of Examples No. 131, 132, 141, 146, 215, 220, 253, 254, 255, 257, 259, 261, 268, 304, 351, 367, 372, 374, and 375. Also particularly preferred are compounds selected from the group consisting of Examples No. 151, 155, 160, 164, 169, 173, 196, 200, 204, 225, 227, 228, 229, 251, 252, 273, 276, 277, 278, 299, 300, 301, 302, 303, 312, 316, 317, 323, 352, 368, and 371.

The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In addition, the present invention embraces all geometric and positional isomers. For example, if a compound of the invention incorporates a double bond or a fused ring, the cis- and trans-forms, as well as mixtures thereof, are embraced within the scope of the invention. Both the single positional isomers and mixture of positional isomers are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embraces both solvated and unsolvated forms.

The compounds of the invention may also exist in different tautomeric forms (tautomers), and all such forms are embraced with the scope of the invention.

The compounds of the invention may be synthesized by synthetic routes that include processes analogous to those well known in the chemical arts, particularly in light of the description contained herein. The starting materials are generally available from commercial sources or are readily prepared using methods well known to those skilled in the art.

For illustrative purposes, Schemes 1-7 show general methods for preparing the compounds of the present invention as well as key intermediates. For a more detailed description of the individual reaction steps, see the examples hereinbelow. Those skilled in the art will appreciate that other synthetic routes may be used to synthesize the compounds of the invention. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In preparing compounds of the invention, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoro-acetyl, t-butoxycarbonyl (BOC), benzyloxycarbonyl (CBz) and 9-fluorenylmethoxycarbonyl (Fmoc). The need for such protection is readily determined by one skilled in the art.

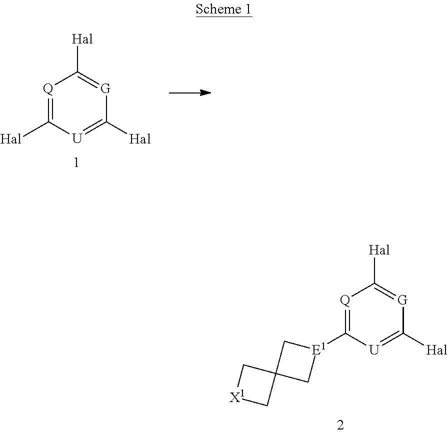

Scheme 1 shows a general method for preparation of the triazine intermediate 2 from 2,4,6-trihalo-1,3,5-triazine reagent (1), wherein Hal is Cl, Br, or I; G=Q=U is N, and E¹ and X¹ are as defined for formula (I), or precursors thereto.

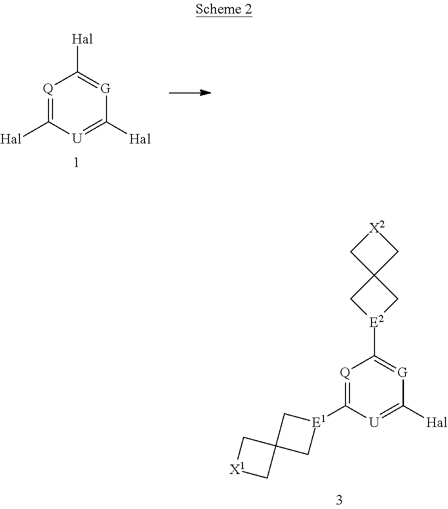

Scheme 2 shows a general method for preparation of the triazine intermediate 3 from 2,4,6-trihalo-1,3,5-triazine reagent (1), wherein Hal is Cl, Br, or I; G=Q=U is N, and E′, E², X¹ and X² are as defined for formula (I), or precursors thereto.

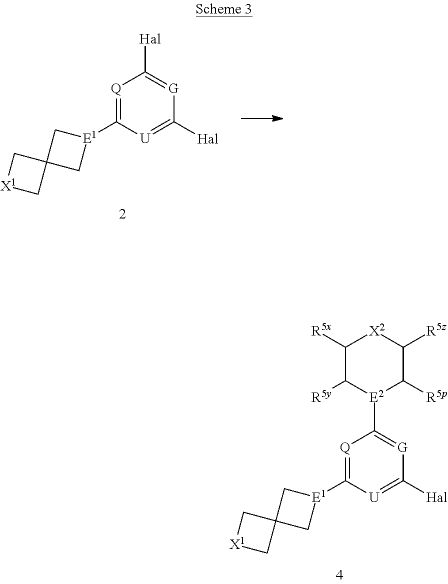

Scheme 3 shows a general method for selectively displacing a halide from bis-halo triazine intermediate 2 with morpholine, a morpholine derivative or a piperazine derivative in an organic solvent to prepare morpholino- or piperazino-triazine intermediate compounds 4, wherein Hal is Cl, Br, or I; G=Q=U is N, E² is N, and E¹, X¹, X², R^{5X}, R^{5y}, R^{5z} and R^{5p} are as defined for formula (I), or precursors thereto.

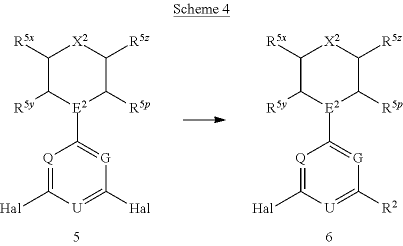

Scheme 4 shows a general method for selectively displacing a halide from intermediate 5 with a heteroaryl secondary amine R²H in an organic solvent to prepare intermediate compounds 6, wherein Hal is Cl, Br or I; G=Q=U is N, E², X², R², R^{5x}, R^{5y}, R^{5z} and R^{5p} are as defined for formula (I), or precursors thereto.

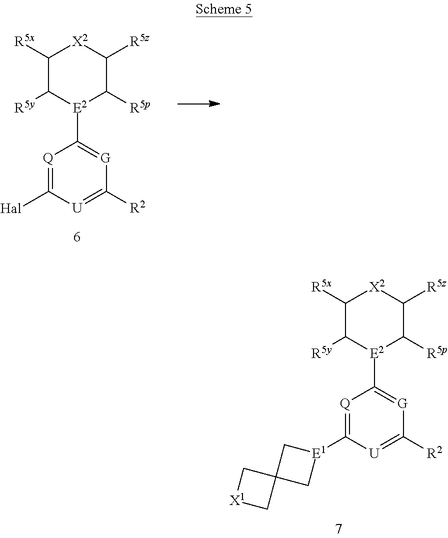

Scheme 5 shows a general method for selectively displacing a halide from intermediate 6 with a specific spirocyclic group in an organic solvent to prepare intermediate compounds 7, wherein Hal is Cl, Br or I; E¹ is N, and E², X¹, X², R², R^{5x}, R^{5y}, R^{5z} and R^{5p} are as defined for formula (I), or precursors or prodrugs thereto.

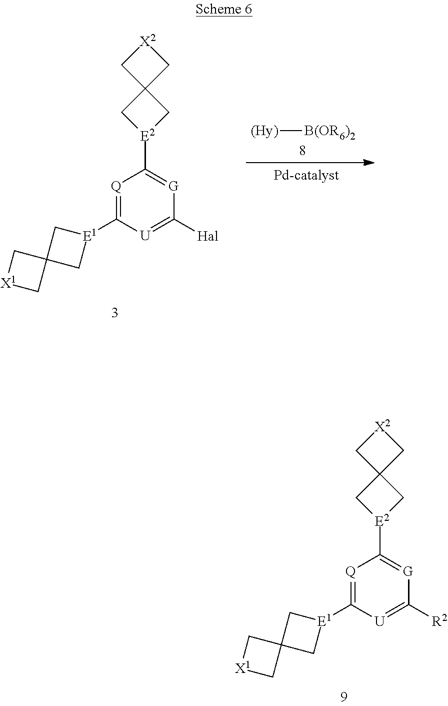

Scheme 6 shows a general method for Suzuki-type coupling of a halotriazine intermediate 3 with a heteroaryl boronic acid (R₆═H) or heteroaryl boronic ester (R₆=alkyl or R₆/R₆=alkylene) reagent 8 to prepare heteroaryl compounds 9, wherein Hal is Cl, Br or I, R₂ is heteroaryl Hy, and E¹, E², X¹ and X² are as defined for formula (I), or precursors or prodrugs thereto. For reviews of the Suzuki reaction, see: Miyaura et al., Chem. Rev. 95:2457-2483 (1995); Suzuki, A., J. Organomet. Chem. 576:147-168 (1999); Suzuki, A. in Metal-Catalyzed Cross-Coupling Reactions, Diederich, F., Stang, P. J., Eds., VCH, Weinheim, Del. (1998), pp 49-97. The palladium catalyst may be any that is typically used for Suzuki-type cross-couplings, such as PdCl₂(PPh₃)₂, Pd(PPh₃)₄, Pd(OAc)₂, PdCl₂(dppf)-DCM, or Pd₂(dba)₃/Pt-Bu)₃.

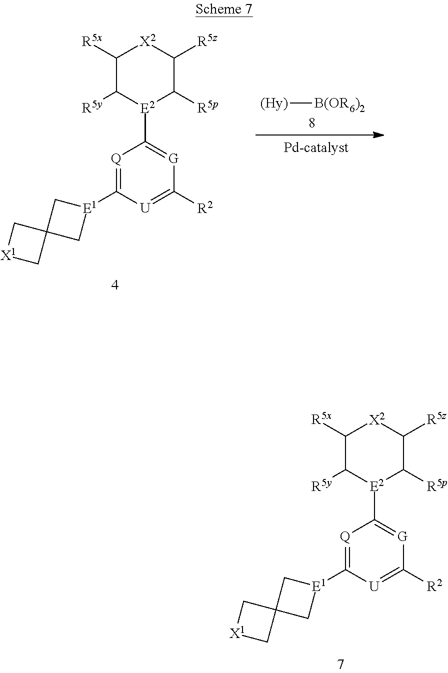

Scheme 7 shows an anaolgous method for Suzuki-type coupling of a halo-morpholino- or piperidino-triazine type intermediate 4 with a heteroaryl boronic acid or ester reagent 8 to prepare the heteroaryl compounds 7, wherein Hal is Cl, Br or I, R² is heteroaryl Hy, and E¹, E², X¹, X², R^{5x}, R^{5y}, R^{5Z} and R^{5p} are as defined for formula (I), or precursors or prodrugs thereto.

### Separation and Purification

In the methods of preparing the compounds of this invention, it may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography. Another class of separation methods involves treatment of a mixture with a reagent selected from activated carbon, molecular sieves, ion exchange media, or the like.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

### Methods of Treatment

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

A dose to treat human patients may range from about 10 mg to about 1000 mg of the compound of the invention. A typical dose may be about 100 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

Compounds of the present invention are useful for treating diseases, conditions and/or disorders including, but not limited to, those characterized by over expression of lipid kinases, e.g. PI3 kinase. Accordingly, another aspect of this invention includes methods of treating or preventing diseases or conditions that can be treated or prevented by inhibiting lipid kinases, including PI3K and mTOR. In one embodiment, the method comprises administering to a mammal in need thereof a therapeutically effective amount of a compound of the invention or of pharmaceutical composition comprising it.

Diseases and conditions treatable according to the methods of this invention include, but are not limited to, cancer, stroke, diabetes, hepatomegaly, cardiovascular disease, Alzheimer's disease, cystic fibrosis, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient.

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

Cardiovascular diseases which can be treated according to the methods of this invention include, but are not limited to, restenosis, cardiomegaly, atherosclerosis, myocardial infarction, and congestive heart failure.

Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Inflammatory diseases which can be treated according to the methods of this invention include, but are not limited to, rheumatoid arthritis, psoriasis, contact dermatitis, and delayed hypersensitivity reactions.

Another aspect of this invention provides a compound of this invention for use in the treatment of the diseases or conditions described herein in a mammal, for example, a human, suffering from such disease or condition. Also provided is the use of a compound of this invention in the preparation of a medicament for the treatment of the diseases and conditions described herein in a warm-blooded animal, such as a mammal, for example a human, suffering from such disorder.

### Pharmaceutical Compositions

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are nontoxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives.

The formulations may be prepared using conventional dissolution and mixing procedures. For example, the bulk drug substance is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of the invention having the desired degree of purity may optionally be mixed with pharmaceutically acceptable diluents, carriers, excipients or stabilizers, in the form of a lyophilized formulation, milled powder, or an aqueous solution, formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound of this invention for use herein is preferably sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution.

The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatine micro-capsules and poly-(methylmethacylate) microcapsules, respectively, in colloidal drug delivery systems (for example, liposomes, albumin microspheres, microemulsions, nano-particles and nanocapsules) or in macroemulsions.

Sustained-release preparations of compounds of the invention may be prepared. Suitable examples of sustained-release preparations include semipermeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or polyvinyl alcohol)), polylactides, copolymers of L-glutamic acid and gamma-ethyl-L-glutamate, non-degradable ethylene-vinyl acetate, degradable lactic acid-glycolic acid copolymers and poly-D-(−)-3-hydroxybutyric acid.

Formulations of a compound of the invention suitable for oral administration may be prepared as discrete units such as pills, capsules, cachets or tablets each containing a predetermined amount of a compound of the invention.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of the invention intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxy groups such as propylene glycol, butane-1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of compounds of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. The aqueous and nonaqueous sterile injection solutions may contain anti-oxidants, buffers, bacteriostats and solutes which render the formulation isotonic with the blood of the intended recipient. Aqueous and non-aqueous sterile suspensions may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The compounds of the invention may be employed alone or in combination with other therapeutic agents for the treatment of a disease or disorder described herein, such as a hyperproliferative disorder (e.g., cancer). In certain embodiments, a compound of the invention combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of the invention such that they do not adversely affect each other. Such compounds are suitably present in combination in amounts that are effective for the purpose intended. In one embodiment, a composition of this invention comprises a compound of the invention in combination with a chemotherapeutic agent such as described herein.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

In a particular embodiment of anti-cancer therapy, a compound of the invention may be combined with other chemotherapeutic, hormonal or antibody agents such as those described herein, as well as combined with surgical therapy and radiotherapy. Combination therapies according to the present invention thus comprise the administration of at least one compound of the invention and the use of at least one other cancer treatment method. The amounts of the compound(s) of the invention and the other pharmaceutically active chemotherapeutic agent(s) and the relative timings of administration will be selected in order to achieve the desired combined therapeutic effect.

### Metabolites

Also falling within the scope of this invention are the in vivo metabolic products of compounds of the invention described herein. Such products may result for example from the oxidation, reduction, hydrolysis, amidation, deamidation, esterification, deesterification, enzymatic cleavage, and the like, of the administered compound. Accordingly, the invention includes metabolites of compounds of the invention including compounds produced by a process comprising contacting a compound of this invention with a mammal for a period of time sufficient to yield a metabolic product thereof.

### Prodrugs

In addition to compounds of the invention, the invention also includes pharmaceutically acceptable prodrugs of such compounds. Prodrugs include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include the 20 naturally occurring amino acids and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone, and tert-butylglycine.

Additional types of prodrugs are also encompassed. For instance, a free carboxyl group of a compound of the invention can be derivatized as an amide or alkyl ester. As another example, compounds of this invention comprising free hydroxy groups may be derivatized as prodrugs by converting the hydroxy group into a group such as, but not limited to, a phosphate ester, hemisuccinate, dimethylaminoacetate, or phosphoryloxy-methoxycarbonyl group. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group may be an alkyl ester optionally substituted with groups including, but not limited to, ether, amine and carboxylic acid functionalities, or where the acyl group is an amino acid ester as described above, are also encompassed. More specific examples include replacement of the hydrogen atom of the alcohol group with a group such as C₁-C₆-alkanoyloxymethyl, 1-(C₁-C₆-alkanoyloxy)ethyl, 1-methyl-1-(C₁-C₆-alkanoyloxy)ethyl, C₁-C₆-alkoxycarbonyloxymethyl, C₁-C₆-alkoxycarbonylaminomethyl, succinoyl, C₁-C₆-alkanoyl, α-amino-C₁-C₄-alkanoyl, arylcarbonyl, substituted α-aminoacetyl or α-aminoacetyl-α-aminoacetyl, wherein each substituted α-aminoacetyl group is independently derived from the naturally occurring L-amino acids, P(O)(OH)₂, —P(O)(C₁-C₆-alkyl-O)₂ or glycosyl (the radical resulting from the removal of a hydroxy group of the hemiacetal form of a carbohydrate).

### Biological Evaluation

Determination of the potential to target PI3K/PI3K-related kinases (PIKK) of a compound of formula (I) is possible by a number of direct and indirect detection methods. Certain exemplary compounds described herein were assayed for their phospho-PKB blocking activity and their in vitro activity against tumor cells. The range of phospho-PKB activities was less than 1 nM (nanomolar) to about 10 μM (micromolar). Other exemplary compounds of the invention had phospho-PKB blocking activity 1-C₅₀ values less than 10 nM. Certain compounds of the invention had tumor cell-based activity 1-C₅₀ values less than 100 nM.

The cytotoxic or cytostatic activity of exemplary compounds of formula (I) was measured by establishing a proliferating mammalian tumor cell lines in a cell culture medium, adding a compound of the invention, culturing the cells for a period from about 6 hours to about 3 days; and measuring cell viability. Cell-based assays were used to measure viability, i.e. proliferation (IC₅₀), cytotoxicity (EC₅₀), and induction of apoptosis (caspase activation).

The in vitro potency of compounds of formula (I) was measured by the in-cell Western assay designed and developed in laboratories at University of Basel. This assay method was conducted in microtiter plate formats, making it amenable to high-throughput screening (HTS). Inhibitors were added to the medium and incubated. Antibodies diluted in PBS/T against pPKB Ser473 (Cell Signalling) and PKB or pS6 Ser 235/236 (Cell Signalling) were incubated overnight and then secondary fluorescently labelled antibodies (LI-COR) were applied and plates were scanned on an Odyssey reader to detect pPKB/PKB ratios.

The following compounds have shown particularly interesting biological activities:

TABLE 1
Compounds of formula (I)

| No. | Q | G | U | R¹ | R² | E¹ | X¹ |
|---|---|---|---|---|---|---|---|
| 100 | N | N | N | 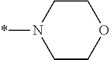 [SMILES: *N1CCOCC1] | 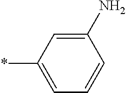 [SMILES: *c1cccc(N)c1] | N | O |
| 101 | N | N | N | 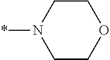 [SMILES: *N1CCOCC1] | 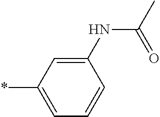 [SMILES: *c1cccc(NC(C)=O)c1] | N | O |
| 102 | N | N | N | 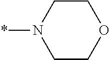 [SMILES: *N1CCOCC1] | 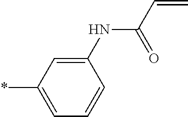 [SMILES: *c1cccc(NC(=O)C=C)c1] | N | O |
| 103 | N | CH | N | 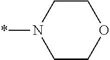 [SMILES: *N1CCOCC1] | 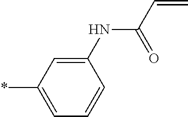 [SMILES: *c1cccc(NC(=O)C=C)c1] | N | O |
| 104 | N | N | N | 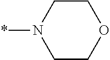 [SMILES: *N1CCOCC1] | 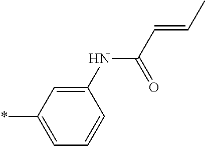 [SMILES: *c1cccc(NC(=O)/C=C/C)c1] | N | O |
| 105 | N | N | N | 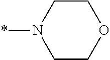 [SMILES: *N1CCOCC1] | 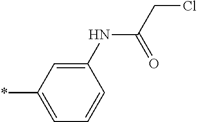 [SMILES: *c1cccc(NC(=O)CCl)c1] | N | O |
| 106 | N | N | N | 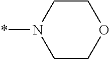 [SMILES: *N1CCOCC1] | 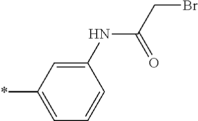 [SMILES: *c1cccc(NC(=O)CBr)c1] | N | O |
| 107 | N | N | N | 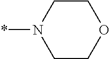 [SMILES: *N1CCOCC1] | 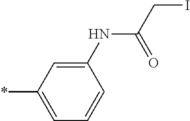 [SMILES: *c1cccc(NC(=O)CI)c1] | N | O |
| 108 | N | N | N | 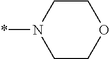 [SMILES: *N1CCOCC1] | 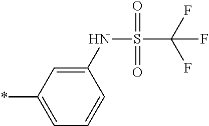 [SMILES: *c1cccc(NS(=O)(=O)C(F)(F)F)c1] | N | O |
| 109 | N | N | N | 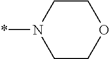 [SMILES: *N1CCOCC1] | 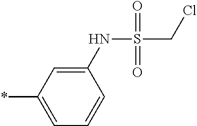 [SMILES: *c1cccc(NS(=O)(=O)CCl)c1] | N | O |
| 110 | N | N | N | 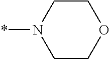 [SMILES: *N1CCOCC1] | 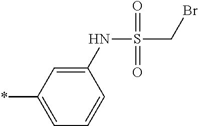 [SMILES: *c1cccc(NS(=O)(=O)CBr)c1] | N | O |
| 111 | N | N | N | 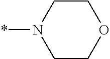 [SMILES: *N1CCOCC1] | 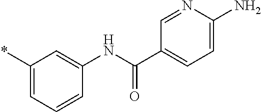 [SMILES: *c1cccc(NC(=O)c2ccc(N)nc2)c1] | N | O |
| 112 | CH | N | N | 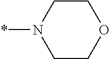 [SMILES: *N1CCOCC1] | 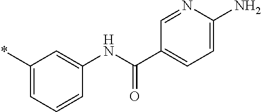 [SMILES: *c1cccc(NC(=O)c2ccc(N)nc2)c1] | N | O |
| 113 | N | CH | N | 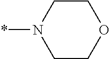 [SMILES: *N1CCOCC1] | 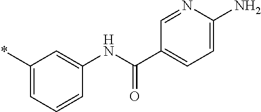 [SMILES: *c1cccc(NC(=O)c2ccc(N)nc2)c1] | N | O |
| 114 | N | N | CH | 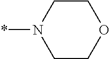 [SMILES: *N1CCOCC1] | 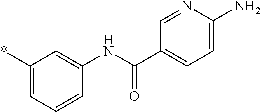 [SMILES: *c1cccc(NC(=O)c2ccc(N)nc2)c1] | N | O |
| 115 | N | N | N | 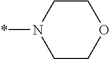 [SMILES: *N1CCOCC1] | 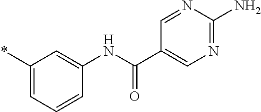 [SMILES: *c1cccc(NC(=O)c2cnc(N)nc2)c1] | N | O |
| 116 | CH | N | N | 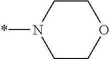 [SMILES: *N1CCOCC1] | 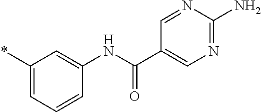 [SMILES: *c1cccc(NC(=O)c2cnc(N)nc2)c1] | N | O |
| 117 | N | CH | N | 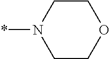 [SMILES: *N1CCOCC1] | 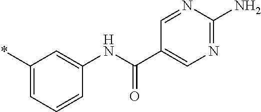 [SMILES: *c1cccc(NC(=O)c2cnc(N)nc2)c1] | N | O |
| 118 | N | N | CH | 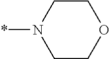 [SMILES: *N1CCOCC1] | 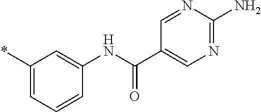 [SMILES: *c1cccc(NC(=O)c2cnc(N)nc2)c1] | N | O |
| 119 | N | N | N | 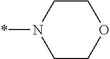 [SMILES: *N1CCOCC1] | 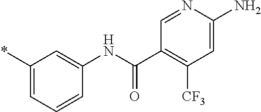 [SMILES: *c1cccc(NC(=O)c2cnc(N)cc2C(F)(F)F)c1] | N | O |
| 120 | CH | N | N | 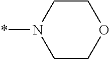 [SMILES: *N1CCOCC1] | 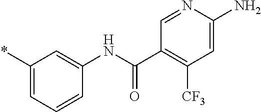 [SMILES: *c1cccc(NC(=O)c2cnc(N)cc2C(F)(F)F)c1] | N | O |
| 121 | N | CH | N | 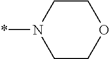 [SMILES: *N1CCOCC1] | 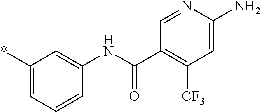 [SMILES: *c1cccc(NC(=O)c2cnc(N)cc2C(F)(F)F)c1] | N | O |
| 122 | N | N | CH | 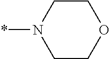 [SMILES: *N1CCOCC1] | 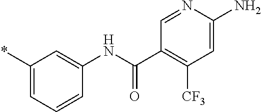 [SMILES: *c1cccc(NC(=O)c2cnc(N)cc2C(F)(F)F)c1] | N | O |
| 123 | N | N | N | 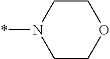 [SMILES: *N1CCOCC1] | 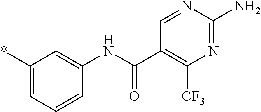 [SMILES: *c1cccc(NC(=O)c2cnc(N)nc2C(F)(F)F)c1] | N | O |
| 124 | N | CH | N | 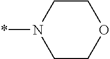 [SMILES: *N1CCOCC1] | 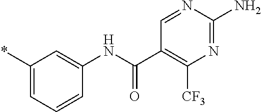 [SMILES: *c1cccc(NC(=O)c2cnc(N)nc2C(F)(F)F)c1] | N | O |
| 125 | N | N | N | 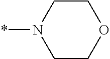 [SMILES: *N1CCOCC1] | 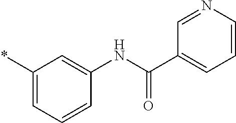 [SMILES: *c1cccc(NC(=O)c2cccnc2)c1] | N | O |
| 126 | CH | N | N | 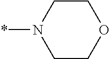 [SMILES: *N1CCOCC1] | 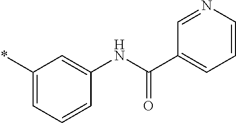 [SMILES: *c1cccc(NC(=O)c2cccnc2)c1] | N | O |
| 127 | N | CH | N | 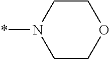 [SMILES: *N1CCOCC1] | 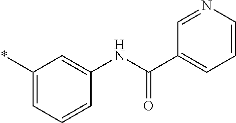 [SMILES: *c1cccc(NC(=O)c2cccnc2)c1] | N | O |
| 128 | N | N | CH | 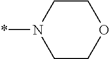 [SMILES: *N1CCOCC1] | 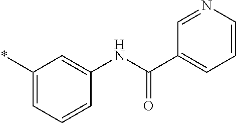 [SMILES: *c1cccc(NC(=O)c2cccnc2)c1] | N | O |
| 129 | N | N | N | 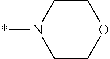 [SMILES: *N1CCOCC1] | 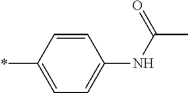 [SMILES: *c1ccc(NC(C)=O)cc1] | N | O |
| 130 | N | N | N | 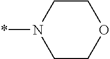 [SMILES: *N1CCOCC1] | 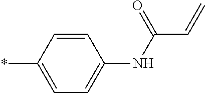 [SMILES: *c1ccc(NC(=O)C=C)cc1] | N | O |
| 131 | N | N | N | 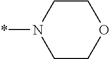 [SMILES: *N1CCOCC1] | 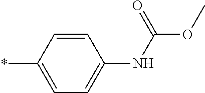 [SMILES: *c1ccc(NC(=O)OC)cc1] | N | O |
| 132 | N | N | N | 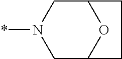 [SMILES: *N1CC2CCC(C1)O2] | 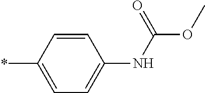 [SMILES: *c1ccc(NC(=O)OC)cc1] | N | O |
| 133 | N | N | N | 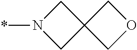 [SMILES: *N1CC2(COC2)C1] | 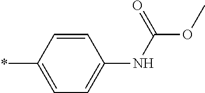 [SMILES: *c1ccc(NC(=O)OC)cc1] | N | O |
| 134 | CH | N | N | 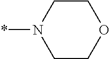 [SMILES: *N1CCOCC1] | 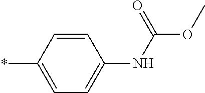 [SMILES: *c1ccc(NC(=O)OC)cc1] | N | O |
| 135 | N | CH | N | 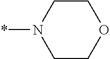 [SMILES: *N1CCOCC1] | 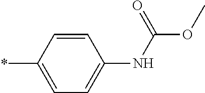 [SMILES: *c1ccc(NC(=O)OC)cc1] | N | O |
| 136 | N | N | CH | 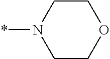 [SMILES: *N1CCOCC1] | 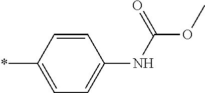 [SMILES: *c1ccc(NC(=O)OC)cc1] | N | O |
| 137 | CH | N | N | 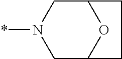 [SMILES: *N1CC2CCC(C1)O2] | 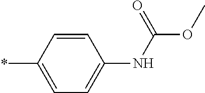 [SMILES: *c1ccc(NC(=O)OC)cc1] | N | O |
| 138 | N | CH | N | 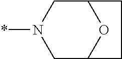 [SMILES: *N1CC2CCC(C1)O2] | 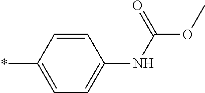 [SMILES: *c1ccc(NC(=O)OC)cc1] | N | O |
| 139 | N | N | CH | 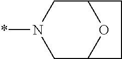 [SMILES: *N1CC2CCC(C1)O2] | 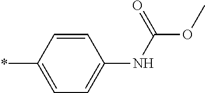 [SMILES: *c1ccc(NC(=O)OC)cc1] | N | O |
| 140 | N | N | N | 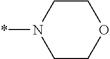 [SMILES: *N1CCOCC1] | 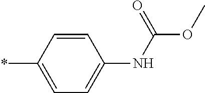 [SMILES: *c1ccc(NC(=O)OC)cc1] | N | NH |
| 141 | N | N | N | 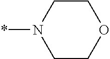 [SMILES: *N1CCOCC1] | 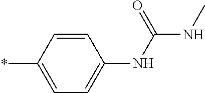 [SMILES: *c1ccc(NC(=O)NC)cc1] | N | O |
| 142 | N | N | N | 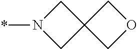 [SMILES: *N1CC2(COC2)C1] | 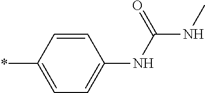 [SMILES: *c1ccc(NC(=O)NC)cc1] | N | O |
| 143 | CH | N | N | 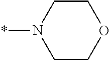 [SMILES: *N1CCOCC1] | 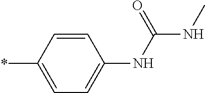 [SMILES: *c1ccc(NC(=O)NC)cc1] | N | O |
| 144 | N | CH | N | 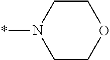 [SMILES: *N1CCOCC1] | 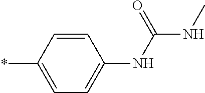 [SMILES: *c1ccc(NC(=O)NC)cc1] | N | O |
| 145 | N | N | CH | 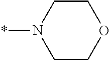 [SMILES: *N1CCOCC1] | 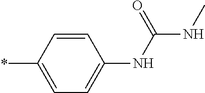 [SMILES: *c1ccc(NC(=O)NC)cc1] | N | O |
| 146 | N | N | N | 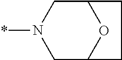 [SMILES: *N1CC2CCC(C1)O2] | 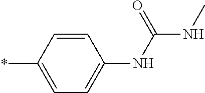 [SMILES: *c1ccc(NC(=O)NC)cc1] | N | O |
| 147 | CH | N | N | 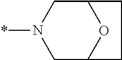 [SMILES: *N1CC2CCC(C1)O2] | 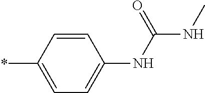 [SMILES: *c1ccc(NC(=O)NC)cc1] | N | O |
| 148 | N | CH | N | 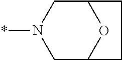 [SMILES: *N1CC2CCC(C1)O2] | 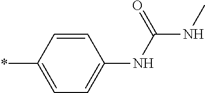 [SMILES: *c1ccc(NC(=O)NC)cc1] | N | O |
| 149 | N | N | CH | 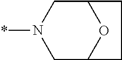 [SMILES: *N1CC2CCC(C1)O2] | 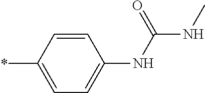 [SMILES: *c1ccc(NC(=O)NC)cc1] | N | O |
| 150 | N | N | N | 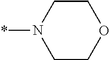 [SMILES: *N1CCOCC1] | 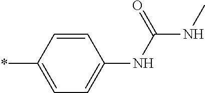 [SMILES: *c1ccc(NC(=O)NC)cc1] | N | NH |
| 151 | N | N | N | 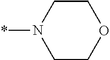 [SMILES: *N1CCOCC1] | 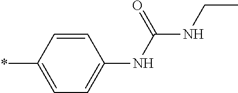 [SMILES: *c1ccc(NC(=O)NCC)cc1] | N | O |
| 152 | CH | N | N | 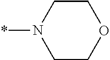 [SMILES: *N1CCOCC1] | 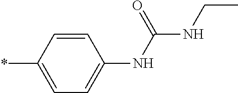 [SMILES: *c1ccc(NC(=O)NCC)cc1] | N | O |
| 153 | N | CH | N | 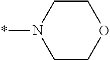 [SMILES: *N1CCOCC1] | 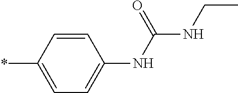 [SMILES: *c1ccc(NC(=O)NCC)cc1] | N | O |
| 154 | N | N | CH | 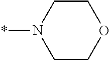 [SMILES: *N1CCOCC1] | 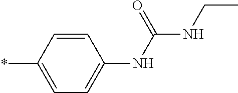 [SMILES: *c1ccc(NC(=O)NCC)cc1] | N | O |
| 155 | N | N | N | 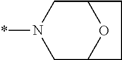 [SMILES: *N1CC2CCC(C1)O2] | 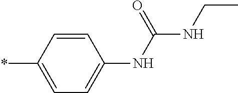 [SMILES: *c1ccc(NC(=O)NCC)cc1] | N | O |
| 156 | CH | N | N | 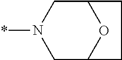 [SMILES: *N1CC2CCC(C1)O2] | 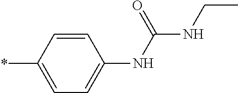 [SMILES: *c1ccc(NC(=O)NCC)cc1] | N | O |
| 157 | N | CH | N | 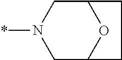 [SMILES: *N1CC2CCC(C1)O2] | 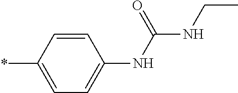 [SMILES: *c1ccc(NC(=O)NCC)cc1] | N | O |
| 158 | N | N | CH | 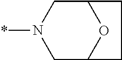 [SMILES: *N1CC2CCC(C1)O2] | 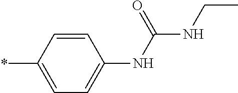 [SMILES: *c1ccc(NC(=O)NCC)cc1] | N | O |
| 159 | N | N | N | 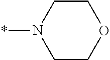 [SMILES: *N1CCOCC1] | 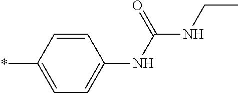 [SMILES: *c1ccc(NC(=O)NCC)cc1] | N | NH |
| 160 | N | N | N | 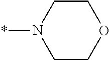 [SMILES: *N1CCOCC1] | 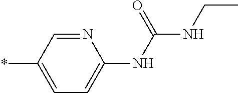 [SMILES: *c1ccc(NC(=O)NCC)nc1] | N | O |
| 161 | CH | N | N | 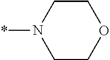 [SMILES: *N1CCOCC1] | 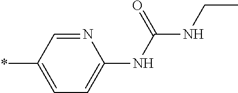 [SMILES: *c1ccc(NC(=O)NCC)nc1] | N | O |
| 162 | N | CH | N | 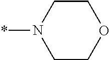 [SMILES: *N1CCOCC1] | 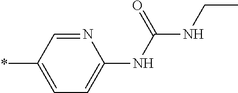 [SMILES: *c1ccc(NC(=O)NCC)nc1] | N | O |
| 163 | N | N | CH | 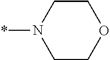 [SMILES: *N1CCOCC1] | 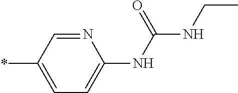 [SMILES: *c1ccc(NC(=O)NCC)nc1] | N | O |
| 164 | N | N | N | 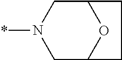 [SMILES: *N1CC2CCC(C1)O2] | 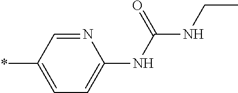 [SMILES: *c1ccc(NC(=O)NCC)nc1] | N | O |
| 165 | CH | N | N | 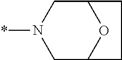 [SMILES: *N1CC2CCC(C1)O2] | 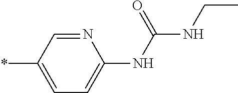 [SMILES: *c1ccc(NC(=O)NCC)nc1] | N | O |
| 166 | N | CH | N | 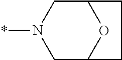 [SMILES: *N1CC2CCC(C1)O2] | 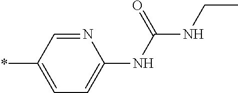 [SMILES: *c1ccc(NC(=O)NCC)nc1] | N | O |
| 167 | N | N | CH | 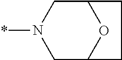 [SMILES: *N1CC2CCC(C1)O2] | 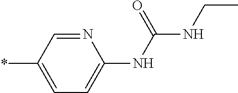 [SMILES: *c1ccc(NC(=O)NCC)nc1] | N | O |
| 168 | N | N | N | 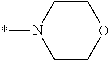 [SMILES: *N1CCOCC1] | 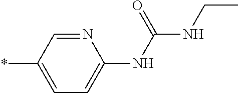 [SMILES: *c1ccc(NC(=O)NCC)nc1] | N | NH |
| 169 | N | N | N | 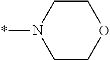 [SMILES: *N1CCOCC1] | 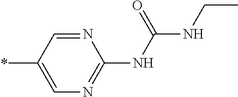 [SMILES: *c1cnc(NC(=O)NCC)nc1] | N | O |
| 170 | CH | N | N | 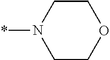 [SMILES: *N1CCOCC1] | 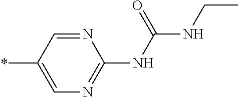 [SMILES: *c1cnc(NC(=O)NCC)nc1] | N | O |
| 171 | N | CH | N | 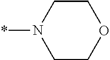 [SMILES: *N1CCOCC1] | 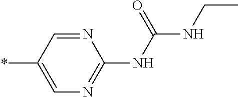 [SMILES: *c1cnc(NC(=O)NCC)nc1] | N | O |
| 172 | N | N | CH | 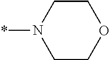 [SMILES: *N1CCOCC1] | 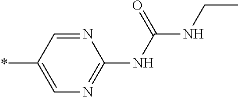 [SMILES: *c1cnc(NC(=O)NCC)nc1] | N | O |
| 173 | N | N | N | 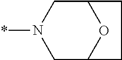 [SMILES: *N1CC2CCC(C1)O2] | 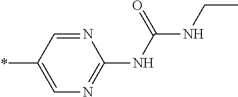 [SMILES: *c1cnc(NC(=O)NCC)nc1] | N | O |
| 174 | CH | N | N | 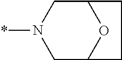 [SMILES: *N1CC2CCC(C1)O2] | 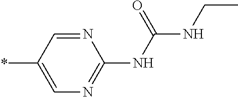 [SMILES: *c1cnc(NC(=O)NCC)nc1] | N | O |
| 175 | N | CH | N | 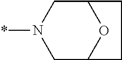 [SMILES: *N1CC2CCC(C1)O2] | 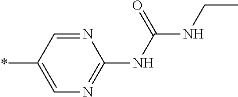 [SMILES: *c1cnc(NC(=O)NCC)nc1] | N | O |
| 176 | N | N | CH | 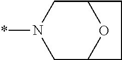 [SMILES: *N1CC2CCC(C1)O2] | 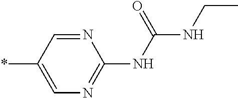 [SMILES: *c1cnc(NC(=O)NCC)nc1] | N | O |
| 177 | N | N | N | 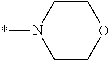 [SMILES: *N1CCOCC1] | 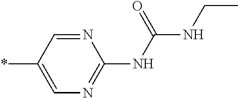 [SMILES: *c1cnc(NC(=O)NCC)nc1] | N | NH |
| 178 | N | N | N | 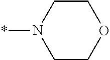 [SMILES: *N1CCOCC1] | 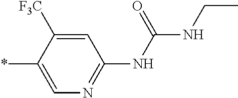 [SMILES: *c1cnc(NC(=O)NCC)cc1C(F)(F)F] | N | O |
| 179 | CH | N | N | 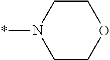 [SMILES: *N1CCOCC1] | 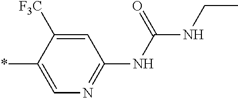 [SMILES: *c1cnc(NC(=O)NCC)cc1C(F)(F)F] | N | O |
| 180 | N | CH | N | 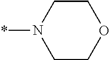 [SMILES: *N1CCOCC1] | 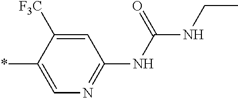 [SMILES: *c1cnc(NC(=O)NCC)cc1C(F)(F)F] | N | O |
| 181 | N | N | CH | 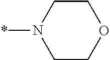 [SMILES: *N1CCOCC1] | 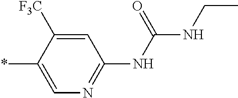 [SMILES: *c1cnc(NC(=O)NCC)cc1C(F)(F)F] | N | O |
| 182 | N | N | N | 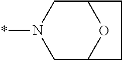 [SMILES: *N1CC2CCC(C1)O2] | 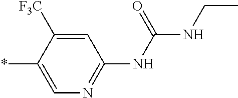 [SMILES: *c1cnc(NC(=O)NCC)cc1C(F)(F)F] | N | O |
| 183 | CH | N | N | 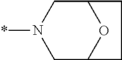 [SMILES: *N1CC2CCC(C1)O2] | 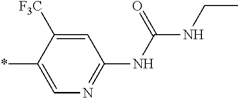 [SMILES: *c1cnc(NC(=O)NCC)cc1C(F)(F)F] | N | O |
| 184 | N | CH | N | 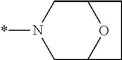 [SMILES: *N1CC2CCC(C1)O2] | 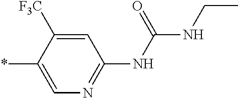 [SMILES: *c1cnc(NC(=O)NCC)cc1C(F)(F)F] | N | O |
| 185 | N | N | CH | 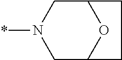 [SMILES: *N1CC2CCC(C1)O2] | 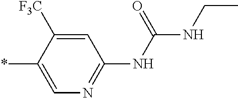 [SMILES: *c1cnc(NC(=O)NCC)cc1C(F)(F)F] | N | O |
| 186 | N | N | N | 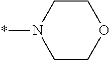 [SMILES: *N1CCOCC1] | 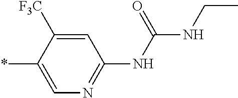 [SMILES: *c1cnc(NC(=O)NCC)cc1C(F)(F)F] | N | NH |
| 187 | N | N | N | 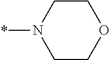 [SMILES: *N1CCOCC1] | 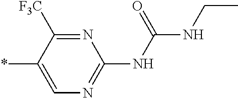 [SMILES: *c1cnc(NC(=O)NCC)nc1C(F)(F)F] | N | O |
| 188 | CH | N | N | 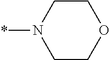 [SMILES: *N1CCOCC1] | 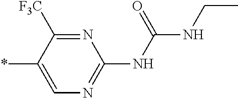 [SMILES: *c1cnc(NC(=O)NCC)nc1C(F)(F)F] | N | O |
| 189 | N | CH | N | 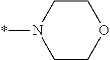 [SMILES: *N1CCOCC1] | 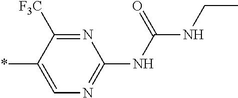 [SMILES: *c1cnc(NC(=O)NCC)nc1C(F)(F)F] | N | O |
| 190 | N | N | CH | 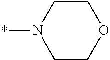 [SMILES: *N1CCOCC1] | 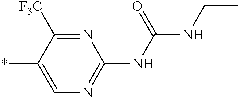 [SMILES: *c1cnc(NC(=O)NCC)nc1C(F)(F)F] | N | O |
| 191 | N | N | N | 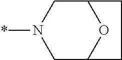 [SMILES: *N1CC2CCC(C1)O2] | 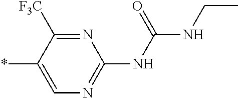 [SMILES: *c1cnc(NC(=O)NCC)nc1C(F)(F)F] | N | O |
| 192 | CH | N | N | 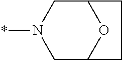 [SMILES: *N1CC2CCC(C1)O2] | 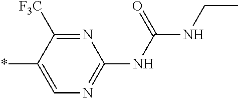 [SMILES: *c1cnc(NC(=O)NCC)nc1C(F)(F)F] | N | O |
| 193 | N | CH | N | 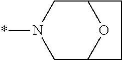 [SMILES: *N1CC2CCC(C1)O2] | 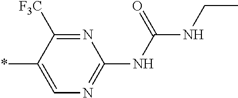 [SMILES: *c1cnc(NC(=O)NCC)nc1C(F)(F)F] | N | O |
| 194 | N | N | CH | 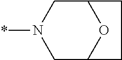 [SMILES: *N1CC2CCC(C1)O2] | 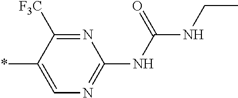 [SMILES: *c1cnc(NC(=O)NCC)nc1C(F)(F)F] | N | O |
| 195 | N | N | N | 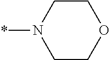 [SMILES: *N1CCOCC1] | 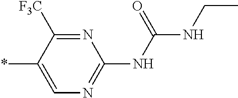 [SMILES: *c1cnc(NC(=O)NCC)nc1C(F)(F)F] | N | NH |
| 196 | N | N | N | 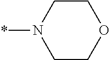 [SMILES: *N1CCOCC1] | 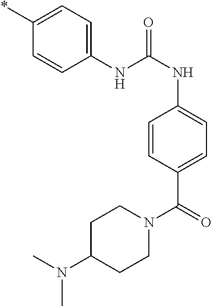 [SMILES: *c1ccc(NC(=O)Nc2ccc(C(=O)N3CCC(N(C)C)CC3)cc2)cc1] | N | O |
| 197 | CH | N | N | 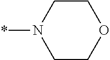 [SMILES: *N1CCOCC1] | 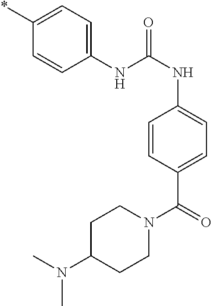 [SMILES: *c1ccc(NC(=O)Nc2ccc(C(=O)N3CCC(N(C)C)CC3)cc2)cc1] | N | O |
| 198 | N | CH | N | 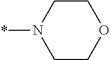 [SMILES: *N1CCOCC1] | 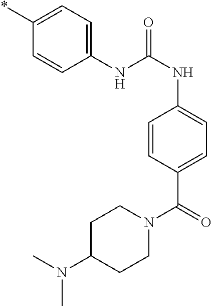 [SMILES: *c1ccc(NC(=O)Nc2ccc(C(=O)N3CCC(N(C)C)CC3)cc2)cc1] | N | O |
| 199 | N | N | CH | 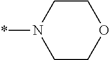 [SMILES: *N1CCOCC1] | 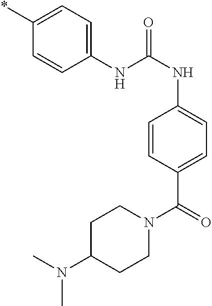 [SMILES: *c1ccc(NC(=O)Nc2ccc(C(=O)N3CCC(N(C)C)CC3)cc2)cc1] | N | O |
| 200 | N | N | N | 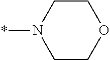 [SMILES: *N1CCOCC1] | 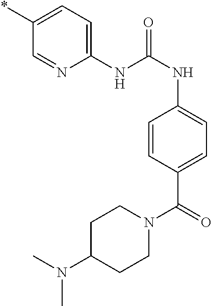 [SMILES: *c1ccc(NC(=O)Nc2ccc(C(=O)N3CCC(N(C)C)CC3)cc2)nc1] | N | O |
| 201 | CH | N | N | 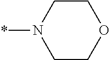 [SMILES: *N1CCOCC1] | 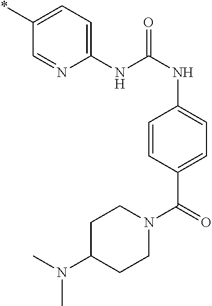 [SMILES: *c1ccc(NC(=O)Nc2ccc(C(=O)N3CCC(N(C)C)CC3)cc2)nc1] | N | O |
| 202 | N | CH | N | 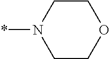 [SMILES: *N1CCOCC1] | 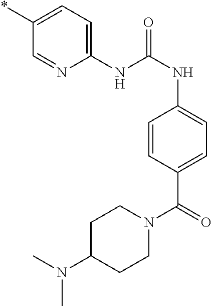 [SMILES: *c1ccc(NC(=O)Nc2ccc(C(=O)N3CCC(N(C)C)CC3)cc2)nc1] | N | O |
| 203 | N | N | CH | 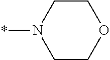 [SMILES: *N1CCOCC1] | 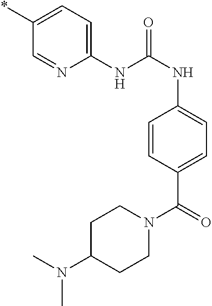 [SMILES: *c1ccc(NC(=O)Nc2ccc(C(=O)N3CCC(N(C)C)CC3)cc2)nc1] | N | O |
| 204 | N | N | N | 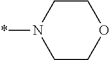 [SMILES: *N1CCOCC1] | 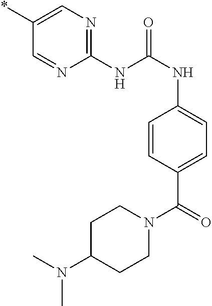 [SMILES: *c1cnc(NC(=O)Nc2ccc(C(=O)N3CCC(N(C)C)CC3)cc2)nc1] | N | O |
| 205 | CH | N | N | 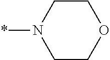 [SMILES: *N1CCOCC1] | 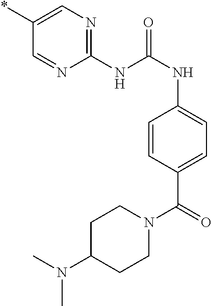 [SMILES: *c1cnc(NC(=O)Nc2ccc(C(=O)N3CCC(N(C)C)CC3)cc2)nc1] | N | O |
| 206 | N | CH | N | 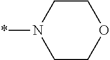 [SMILES: *N1CCOCC1] | 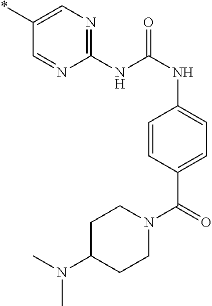 [SMILES: *c1cnc(NC(=O)Nc2ccc(C(=O)N3CCC(N(C)C)CC3)cc2)nc1] | N | O |
| 207 | N | N | N | 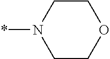 [SMILES: *N1CCOCC1] | 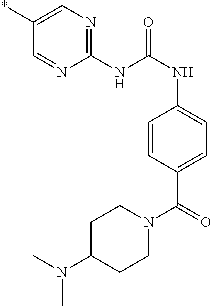 [SMILES: *c1cnc(NC(=O)Nc2ccc(C(=O)N3CCC(N(C)C)CC3)cc2)nc1] | N | O |
| 208 | N | N | N | 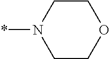 [SMILES: *N1CCOCC1] | 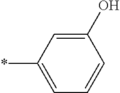 [SMILES: *c1cccc(O)c1] | N | O |
| 209 | N | N | N | 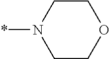 [SMILES: *N1CCOCC1] | 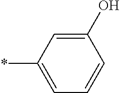 [SMILES: *c1cccc(O)c1] | N | 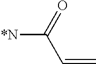 |
| 210 | N | N | N | 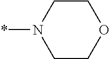 [SMILES: *N1CCOCC1] | 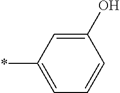 [SMILES: *c1cccc(O)c1] | N | 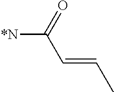 |
| 211 | N | N | N | 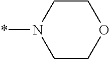 [SMILES: *N1CCOCC1] | 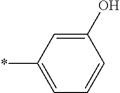 [SMILES: *c1cccc(O)c1] | N | 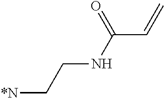 |
| 212 | CH | N | N | 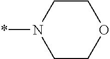 [SMILES: *N1CCOCC1] | 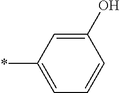 [SMILES: *c1cccc(O)c1] | N | O |
| 213 | N | CH | N | 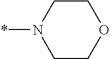 [SMILES: *N1CCOCC1] | 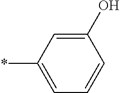 [SMILES: *c1cccc(O)c1] | N | O |
| 214 | N | N | CH | 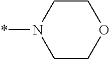 [SMILES: *N1CCOCC1] | 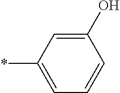 [SMILES: *c1cccc(O)c1] | N | O |
| 215 | N | N | N | 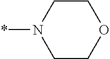 [SMILES: *N1CCOCC1] | 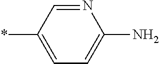 [SMILES: *c1ccc(N)nc1] | N | O |
| 216 | N | N | N | 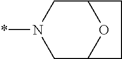 [SMILES: *N1CC2CCC(C1)O2] | 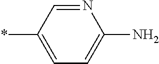 [SMILES: *c1ccc(N)nc1] | N | O |
| 217 | CH | N | N | 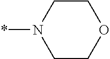 [SMILES: *N1CCOCC1] | 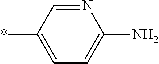 [SMILES: *c1ccc(N)nc1] | N | O |
| 218 | N | CH | N | 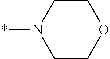 [SMILES: *N1CCOCC1] | 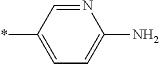 [SMILES: *c1ccc(N)nc1] | N | O |
| 219 | N | N | CH | 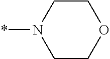 [SMILES: *N1CCOCC1] | 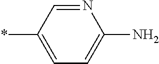 [SMILES: *c1ccc(N)nc1] | N | O |
| 220 | N | N | N | 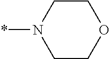 [SMILES: *N1CCOCC1] | 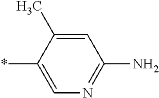 [SMILES: *c1cnc(N)cc1C] | N | O |
| 221 | N | N | N | 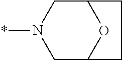 [SMILES: *N1CC2CCC(C1)O2] | 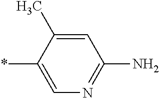 [SMILES: *c1cnc(N)cc1C] | N | O |
| 222 | CH | N | N | 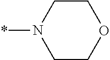 [SMILES: *N1CCOCC1] | 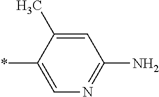 [SMILES: *c1cnc(N)cc1C] | N | O |
| 223 | N | CH | N | 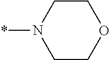 [SMILES: *N1CCOCC1] | 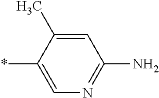 [SMILES: *c1cnc(N)cc1C] | N | O |
| 224 | N | N | CH | 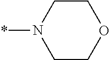 [SMILES: *N1CCOCC1] | 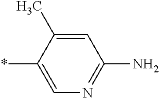 [SMILES: *c1cnc(N)cc1C] | N | O |
| 225 | N | N | N | 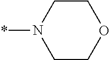 [SMILES: *N1CCOCC1] | 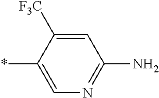 [SMILES: *c1cnc(N)cc1C(F)(F)F] | N | O |
| 226 | N | N | N | 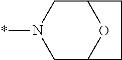 [SMILES: *N1CC2CCC(C1)O2] | 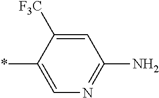 [SMILES: *c1cnc(N)cc1C(F)(F)F] | N | O |
| 227 | CH | N | N | 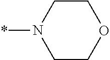 [SMILES: *N1CCOCC1] | 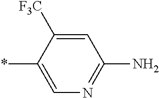 [SMILES: *c1cnc(N)cc1C(F)(F)F] | N | O |
| 228 | N | CH | N | 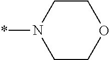 [SMILES: *N1CCOCC1] | 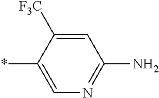 [SMILES: *c1cnc(N)cc1C(F)(F)F] | N | O |
| 229 | N | N | CH | 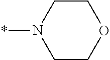 [SMILES: *N1CCOCC1] | 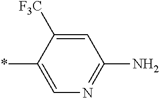 [SMILES: *c1cnc(N)cc1C(F)(F)F] | N | O |
| 230 | N | N | N | 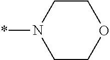 [SMILES: *N1CCOCC1] | 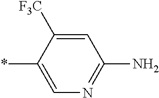 [SMILES: *c1cnc(N)cc1C(F)(F)F] | N | H |
| 231 | N | N | N | 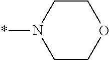 [SMILES: *N1CCOCC1] | 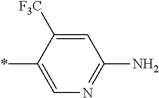 [SMILES: *c1cnc(N)cc1C(F)(F)F] | N | *N—CH₃ |
| 232 | N | N | N | 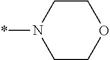 [SMILES: *N1CCOCC1] | 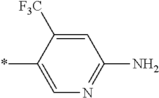 [SMILES: *c1cnc(N)cc1C(F)(F)F] | N | *NSO₂CH₃ |
| 233 | N | N | N | 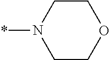 [SMILES: *N1CCOCC1] | 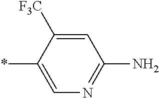 [SMILES: *c1cnc(N)cc1C(F)(F)F] | N | 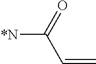 |
| 234 | N | N | N | 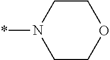 [SMILES: *N1CCOCC1] | 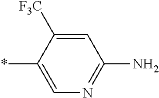 [SMILES: *c1cnc(N)cc1C(F)(F)F] | N | 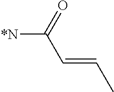 |
| 235 | N | N | N | 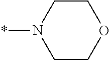 [SMILES: *N1CCOCC1] | 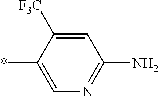 [SMILES: *c1cnc(N)cc1C(F)(F)F] | N | 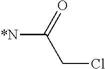 |
| 236 | N | N | N | 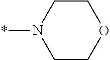 [SMILES: *N1CCOCC1] | 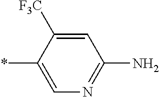 [SMILES: *c1cnc(N)cc1C(F)(F)F] | N | 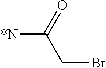 |
| 237 | N | N | N | 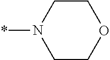 [SMILES: *N1CCOCC1] | 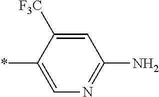 [SMILES: *c1cnc(N)cc1C(F)(F)F] | N | 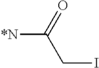 |
| 238 | N | N | N | 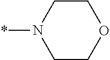 [SMILES: *N1CCOCC1] | 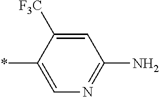 [SMILES: *c1cnc(N)cc1C(F)(F)F] | N | 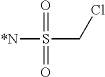 |
| 239 | N | N | N | 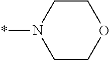 [SMILES: *N1CCOCC1] | 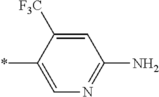 [SMILES: *c1cnc(N)cc1C(F)(F)F] | N | 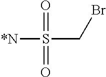 |
| 240 | N | N | N | 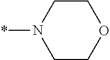 [SMILES: *N1CCOCC1] | 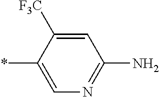 [SMILES: *c1cnc(N)cc1C(F)(F)F] | N | 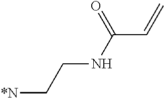 |
| 241 | N | N | N | 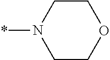 [SMILES: *N1CCOCC1] | 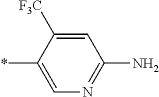 [SMILES: *c1cnc(N)cc1C(F)(F)F] | N | 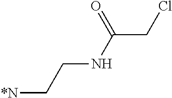 |
| 242 | N | CH | N | 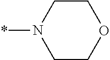 [SMILES: *N1CCOCC1] | 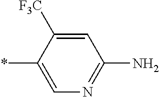 [SMILES: *c1cnc(N)cc1C(F)(F)F] | N | 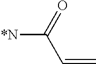 |
| 243 | N | CH | N | 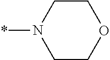 [SMILES: *N1CCOCC1] | 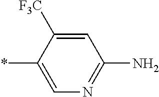 [SMILES: *c1cnc(N)cc1C(F)(F)F] | N | 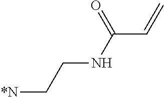 |
| 244 | N | CH | N | 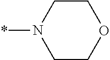 [SMILES: *N1CCOCC1] | 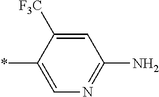 [SMILES: *c1cnc(N)cc1C(F)(F)F] | N | 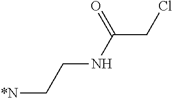 |
| 245 | N | CH | N | 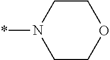 [SMILES: *N1CCOCC1] | 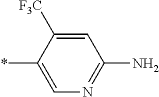 [SMILES: *c1cnc(N)cc1C(F)(F)F] | N | 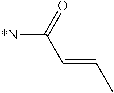 |
| 246 | N | CH | N | 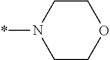 [SMILES: *N1CCOCC1] | 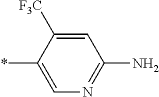 [SMILES: *c1cnc(N)cc1C(F)(F)F] | N | 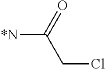 |
| 247 | N | CH | N | 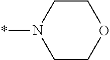 [SMILES: *N1CCOCC1] | 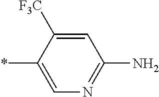 [SMILES: *c1cnc(N)cc1C(F)(F)F] | N | 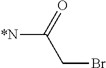 |
| 248 | N | CH | N | 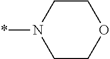 [SMILES: *N1CCOCC1] | 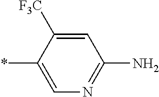 [SMILES: *c1cnc(N)cc1C(F)(F)F] | N | 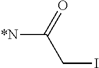 |
| 249 | N | CH | N | 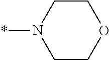 [SMILES: *N1CCOCC1] | 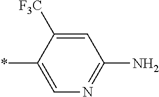 [SMILES: *c1cnc(N)cc1C(F)(F)F] | N | 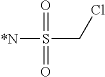 |
| 250 | N | CH | N | 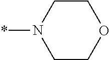 [SMILES: *N1CCOCC1] | 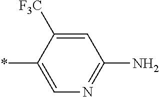 [SMILES: *c1cnc(N)cc1C(F)(F)F] | N | 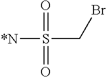 |
| 251 | N | N | N | 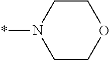 [SMILES: *N1CCOCC1] | 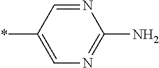 [SMILES: *c1cnc(N)nc1] | N | O |
| 252 | N | N | N | 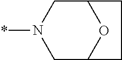 [SMILES: *N1CC2CCC(C1)O2] | 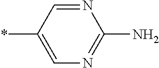 [SMILES: *c1cnc(N)nc1] | N | O |
| 253 | CH | N | N | 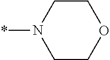 [SMILES: *N1CCOCC1] | 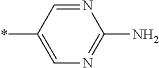 [SMILES: *c1cnc(N)nc1] | N | O |
| 254 | N | CH | N | 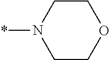 [SMILES: *N1CCOCC1] | 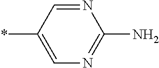 [SMILES: *c1cnc(N)nc1] | N | O |
| 255 | N | N | CH | 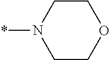 [SMILES: *N1CCOCC1] | 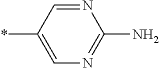 [SMILES: *c1cnc(N)nc1] | N | O |
| 256 | N | N | N | 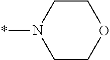 [SMILES: *N1CCOCC1] | 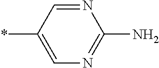 [SMILES: *c1cnc(N)nc1] | N | NH |
| 257 | N | N | N | 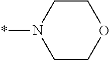 [SMILES: *N1CCOCC1] | 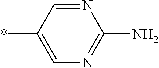 [SMILES: *c1cnc(N)nc1] | N | *N—CH₃ |
| 258 | N | N | N | 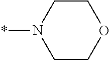 [SMILES: *N1CCOCC1] | 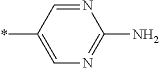 [SMILES: *c1cnc(N)nc1] | N | *NSO₂CH₃ |
| 259 | N | N | N | 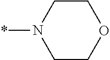 [SMILES: *N1CCOCC1] | 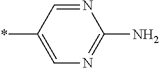 [SMILES: *c1cnc(N)nc1] | N | 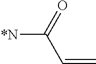 |
| 260 | N | N | N | 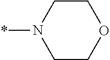 [SMILES: *N1CCOCC1] | 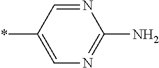 [SMILES: *c1cnc(N)nc1] | N | 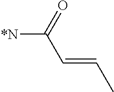 |
| 261 | N | N | N | 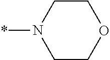 [SMILES: *N1CCOCC1] | 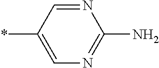 [SMILES: *c1cnc(N)nc1] | N | 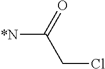 |
| 262 | N | N | N | 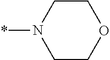 [SMILES: *N1CCOCC1] | 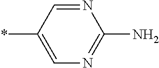 [SMILES: *c1cnc(N)nc1] | N | 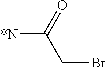 |
| 263 | N | N | N | 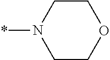 [SMILES: *N1CCOCC1] | 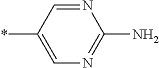 [SMILES: *c1cnc(N)nc1] | N | 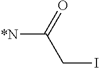 |
| 264 | N | N | N | 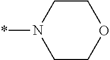 [SMILES: *N1CCOCC1] | 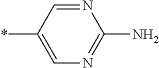 [SMILES: *c1cnc(N)nc1] | N | 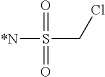 |
| 265 | N | N | N | 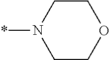 [SMILES: *N1CCOCC1] | 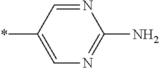 [SMILES: *c1cnc(N)nc1] | N | 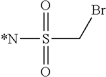 |
| 266 | N | N | N | 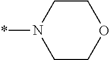 [SMILES: *N1CCOCC1] | 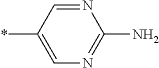 [SMILES: *c1cnc(N)nc1] | N | 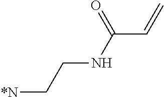 |
| 267 | N | N | N | 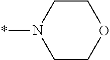 [SMILES: *N1CCOCC1] | 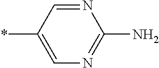 [SMILES: *c1cnc(N)nc1] | N | 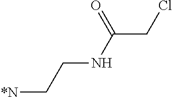 |
| 268 | N | N | N | 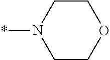 [SMILES: *N1CCOCC1] | 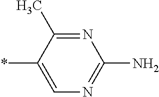 [SMILES: *c1cnc(N)nc1C] | N | O |
| 269 | N | N | N | 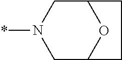 [SMILES: *N1CC2CCC(C1)O2] | 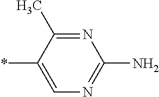 [SMILES: *c1cnc(N)nc1C] | N | O |
| 270 | CH | N | N | 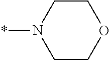 [SMILES: *N1CCOCC1] | 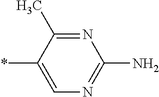 [SMILES: *c1cnc(N)nc1C] | N | O |
| 271 | N | CH | N | 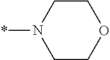 [SMILES: *N1CCOCC1] | 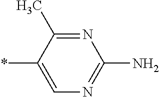 [SMILES: *c1cnc(N)nc1C] | N | O |
| 272 | N | N | CH | 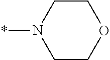 [SMILES: *N1CCOCC1] | 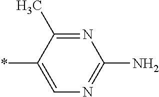 [SMILES: *c1cnc(N)nc1C] | N | O |
| 273 | N | N | N | 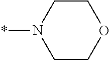 [SMILES: *N1CCOCC1] | 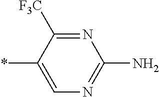 [SMILES: *c1cnc(N)nc1C(F)(F)F] | N | O |
| 274 | N | N | N | 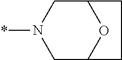 [SMILES: *N1CC2CCC(C1)O2] | 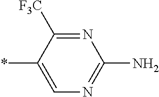 [SMILES: *c1cnc(N)nc1C(F)(F)F] | N | O |
| 275 | N | N | N | 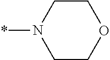 [SMILES: *N1CCOCC1] | 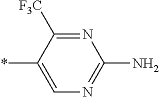 [SMILES: *c1cnc(N)nc1C(F)(F)F] | N | NH |
| 276 | CH | N | N | 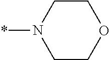 [SMILES: *N1CCOCC1] | 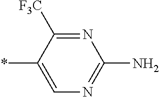 [SMILES: *c1cnc(N)nc1C(F)(F)F] | N | O |
| 277 | N | CH | N | 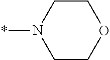 [SMILES: *N1CCOCC1] | 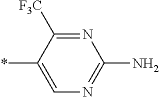 [SMILES: *c1cnc(N)nc1C(F)(F)F] | N | O |
| 278 | N | N | CH | 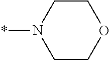 [SMILES: *N1CCOCC1] | 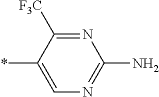 [SMILES: *c1cnc(N)nc1C(F)(F)F] | N | O |
| 279 | N | N | N | 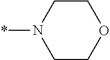 [SMILES: *N1CCOCC1] | 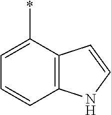 [SMILES: *c1cccc2[nH]ccc12] | N | O |
| 280 | N | N | N | 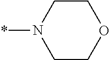 [SMILES: *N1CCOCC1] | 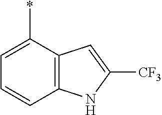 [SMILES: *c1cccc2[nH]c(C(F)(F)F)cc12] | N | O |
| 281 | N | N | N | 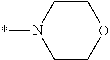 [SMILES: *N1CCOCC1] | 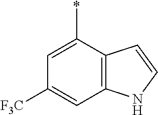 [SMILES: *c1cc(C(F)(F)F)cc2[nH]ccc12] | N | O |
| 282 | CH | N | N | 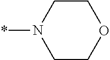 [SMILES: *N1CCOCC1] | 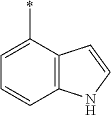 [SMILES: *c1cccc2[nH]ccc12] | N | O |
| 283 | N | CH | N | 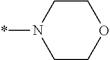 [SMILES: *N1CCOCC1] | 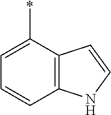 [SMILES: *c1cccc2[nH]ccc12] | N | O |
| 284 | N | N | CH | 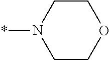 [SMILES: *N1CCOCC1] | 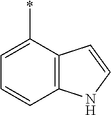 [SMILES: *c1cccc2[nH]ccc12] | N | O |
| 285 | N | N | N | 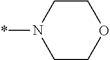 [SMILES: *N1CCOCC1] | 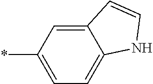 [SMILES: *c1ccc2[nH]ccc2c1] | N | O |
| 286 | N | N | N | 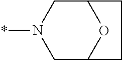 [SMILES: *N1CC2CCC(C1)O2] | 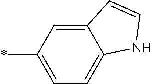 [SMILES: *c1ccc2[nH]ccc2c1] | N | O |
| 287 | CH | N | N | 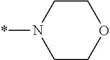 [SMILES: *N1CCOCC1] | 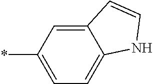 [SMILES: *c1ccc2[nH]ccc2c1] | N | O |
| 288 | CH | N | N | 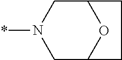 [SMILES: *N1CC2CCC(C1)O2] | 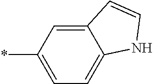 [SMILES: *c1ccc2[nH]ccc2c1] | N | O |
| 289 | N | CH | N | 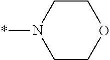 [SMILES: *N1CCOCC1] | 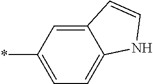 [SMILES: *c1ccc2[nH]ccc2c1] | N | O |
| 290 | N | CH | N | 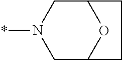 [SMILES: *N1CC2CCC(C1)O2] | 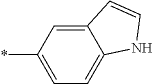 [SMILES: *c1ccc2[nH]ccc2c1] | N | O |
| 291 | N | N | CH | 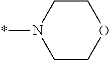 [SMILES: *N1CCOCC1] | 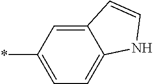 [SMILES: *c1ccc2[nH]ccc2c1] | N | O |
| 292 | N | N | CH | 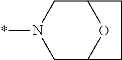 [SMILES: *N1CC2CCC(C1)O2] | 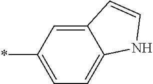 [SMILES: *c1ccc2[nH]ccc2c1] | N | O |
| 293 | N | N | N | 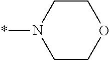 [SMILES: *N1CCOCC1] | 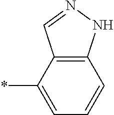 [SMILES: *c1cccc2[nH]ncc12] | N | O |
| 294 | N | N | N | 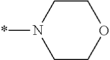 [SMILES: *N1CCOCC1] | 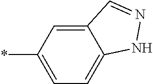 [SMILES: *c1ccc2[nH]ncc2c1] | N | O |
| 295 | N | N | N | 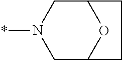 [SMILES: *N1CC2CCC(C1)O2] | 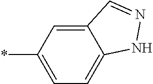 [SMILES: *c1ccc2[nH]ncc2c1] | N | O |
| 296 | CH | N | N | 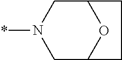 [SMILES: *N1CC2CCC(C1)O2] | 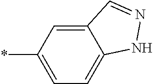 [SMILES: *c1ccc2[nH]ncc2c1] | N | O |
| 297 | N | CH | N | 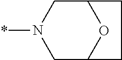 [SMILES: *N1CC2CCC(C1)O2] | 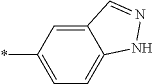 [SMILES: *c1ccc2[nH]ncc2c1] | N | O |
| 298 | N | N | CH | 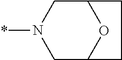 [SMILES: *N1CC2CCC(C1)O2] | 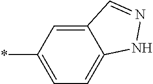 [SMILES: *c1ccc2[nH]ncc2c1] | N | O |
| 299 | N | N | N | 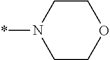 [SMILES: *N1CCOCC1] | 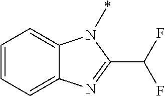 [SMILES: *n1c(C(F)F)nc2ccccc21] | N | O |
| 300 | CH | N | N | 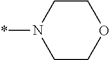 [SMILES: *N1CCOCC1] | 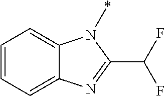 [SMILES: *n1c(C(F)F)nc2ccccc21] | N | O |
| 301 | N | CH | N | 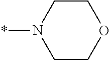 [SMILES: *N1CCOCC1] | 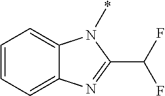 [SMILES: *n1c(C(F)F)nc2ccccc21] | N | O |
| 302 | N | N | CH | 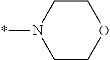 [SMILES: *N1CCOCC1] | 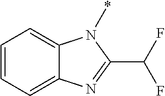 [SMILES: *n1c(C(F)F)nc2ccccc21] | N | O |
| 303 | N | N | N | 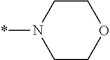 [SMILES: *N1CCOCC1] | 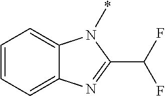 [SMILES: *n1c(C(F)F)nc2ccccc21] | N | NH |
| 304 | N | N | N | 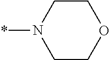 [SMILES: *N1CCOCC1] | 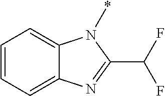 [SMILES: *n1c(C(F)F)nc2ccccc21] | N | *N—CH₃ |
| 305 | N | N | N | 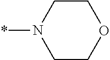 [SMILES: *N1CCOCC1] | 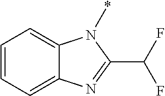 [SMILES: *n1c(C(F)F)nc2ccccc21] | N | *NSO₂CH₃ |
| 306 | N | N | N | 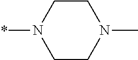 [SMILES: *N1CCN(C)CC1] | 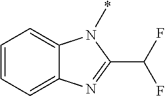 [SMILES: *n1c(C(F)F)nc2ccccc21] | N | O |
| 307 | N | N | N | 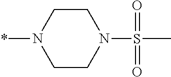 [SMILES: *N1CCN(S(C)(=O)=O)CC1] | 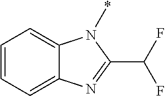 [SMILES: *n1c(C(F)F)nc2ccccc21] | N | O |
| 308 | N | N | N | 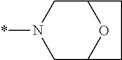 [SMILES: *N1CC2CCC(C1)O2] | 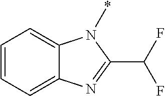 [SMILES: *n1c(C(F)F)nc2ccccc21] | N | O |
| 309 | CH | N | N | 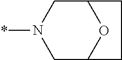 [SMILES: *N1CC2CCC(C1)O2] | 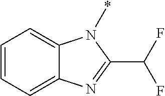 [SMILES: *n1c(C(F)F)nc2ccccc21] | N | O |
| 310 | N | CH | N | 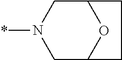 [SMILES: *N1CC2CCC(C1)O2] | 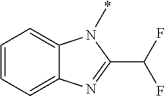 [SMILES: *n1c(C(F)F)nc2ccccc21] | N | O |
| 311 | N | N | CH | 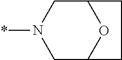 [SMILES: *N1CC2CCC(C1)O2] | 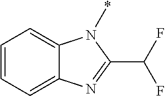 [SMILES: *n1c(C(F)F)nc2ccccc21] | N | O |
| 312 | N | N | N | 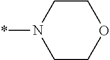 [SMILES: *N1CCOCC1] | 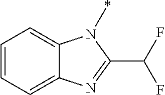 [SMILES: *n1c(C(F)F)nc2ccccc21] | N | 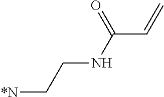 |
| 313 | CH | N | N | 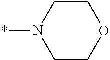 [SMILES: *N1CCOCC1] | 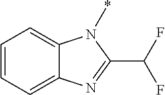 [SMILES: *n1c(C(F)F)nc2ccccc21] | N | 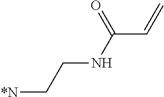 |
| 314 | N | CH | N | 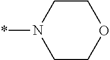 [SMILES: *N1CCOCC1] | 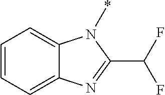 [SMILES: *n1c(C(F)F)nc2ccccc21] | N | 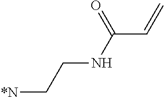 |
| 315 | N | N | CH | 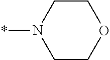 [SMILES: *N1CCOCC1] | 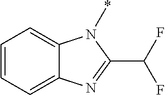 [SMILES: *n1c(C(F)F)nc2ccccc21] | N | 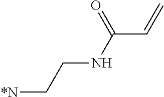 |
| 316 | N | N | N | 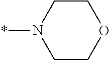 [SMILES: *N1CCOCC1] | 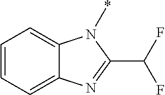 [SMILES: *n1c(C(F)F)nc2ccccc21] | N | 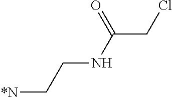 |
| 317 | N | N | N | 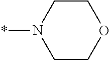 [SMILES: *N1CCOCC1] | 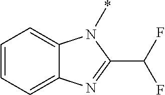 [SMILES: *n1c(C(F)F)nc2ccccc21] | N | 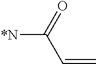 |
| 318 | CH | N | N | 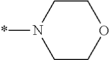 [SMILES: *N1CCOCC1] | 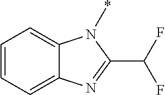 [SMILES: *n1c(C(F)F)nc2ccccc21] | N | 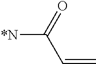 |
| 319 | N | CH | N | 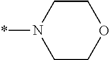 [SMILES: *N1CCOCC1] | 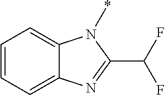 [SMILES: *n1c(C(F)F)nc2ccccc21] | N | 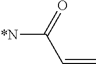 |
| 320 | N | N | CH | 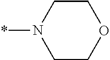 [SMILES: *N1CCOCC1] | 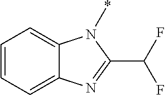 [SMILES: *n1c(C(F)F)nc2ccccc21] | N | 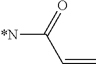 |
| 321 | N | N | N | 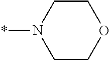 [SMILES: *N1CCOCC1] | 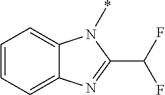 [SMILES: *n1c(C(F)F)nc2ccccc21] | N | 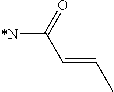 |
| 322 | N | N | N | 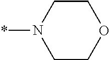 [SMILES: *N1CCOCC1] | 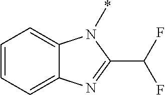 [SMILES: *n1c(C(F)F)nc2ccccc21] | N | 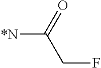 |
| 323 | N | N | N | 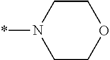 [SMILES: *N1CCOCC1] | 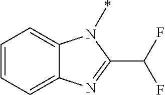 [SMILES: *n1c(C(F)F)nc2ccccc21] | N | 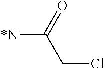 |
| 324 | CH | N | N | 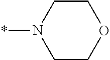 [SMILES: *N1CCOCC1] | 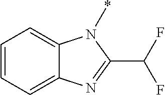 [SMILES: *n1c(C(F)F)nc2ccccc21] | N | 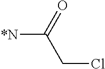 |
| 325 | N | CH | N | 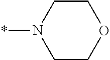 [SMILES: *N1CCOCC1] | 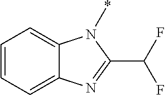 [SMILES: *n1c(C(F)F)nc2ccccc21] | N | 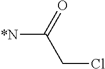 |
| 326 | N | N | CH | 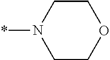 [SMILES: *N1CCOCC1] | 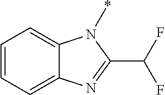 [SMILES: *n1c(C(F)F)nc2ccccc21] | N | 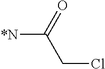 |
| 327 | N | N | N | 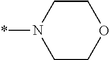 [SMILES: *N1CCOCC1] | 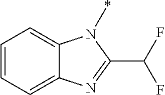 [SMILES: *n1c(C(F)F)nc2ccccc21] | N | 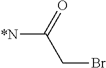 |
| 328 | N | N | N | 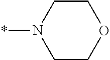 [SMILES: *N1CCOCC1] | 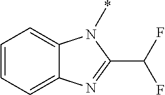 [SMILES: *n1c(C(F)F)nc2ccccc21] | N | 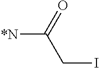 |
| 329 | N | N | N | 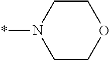 [SMILES: *N1CCOCC1] | 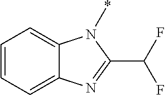 [SMILES: *n1c(C(F)F)nc2ccccc21] | N | 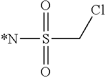 |
| 330 | N | N | N | 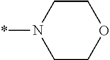 [SMILES: *N1CCOCC1] | 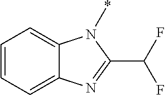 [SMILES: *n1c(C(F)F)nc2ccccc21] | N | 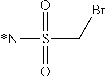 |
| 331 | N | N | N | 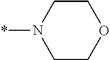 [SMILES: *N1CCOCC1] | 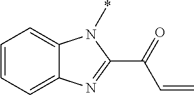 [SMILES: *n1c(C(=O)C=C)nc2ccccc21] | N | O |
| 332 | N | N | N | 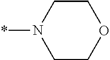 [SMILES: *N1CCOCC1] | 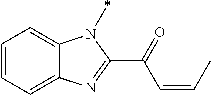 [SMILES: *n1c(C(=O)/C=C\C)nc2ccccc21] | N | O |
| 333 | N | N | N | 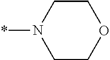 [SMILES: *N1CCOCC1] | 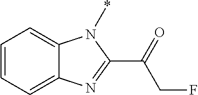 [SMILES: *n1c(C(=O)CF)nc2ccccc21] | N | O |
| 334 | N | N | N | 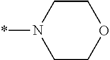 [SMILES: *N1CCOCC1] | 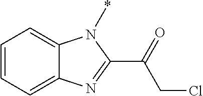 [SMILES: *n1c(C(=O)CCl)nc2ccccc21] | N | O |
| 335 | N | N | N | 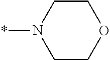 [SMILES: *N1CCOCC1] | 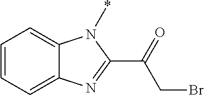 [SMILES: *n1c(C(=O)CBr)nc2ccccc21] | N | O |
| 336 | N | N | N | 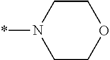 [SMILES: *N1CCOCC1] | 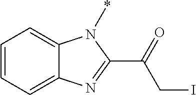 [SMILES: *n1c(C(=O)CI)nc2ccccc21] | N | O |
| 337 | N | N | N | 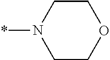 [SMILES: *N1CCOCC1] | 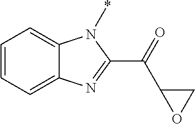 [SMILES: *n1c(C(=O)C2CO2)nc2ccccc21] | N | O |
| 338 | N | N | N | 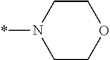 [SMILES: *N1CCOCC1] | 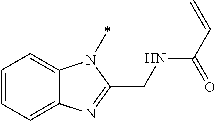 [SMILES: *n1c(CNC(=O)C=C)nc2ccccc21] | N | O |
| 339 | N | N | N | 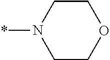 [SMILES: *N1CCOCC1] | 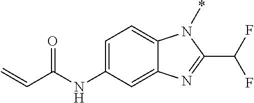 [SMILES: *n1c(C(F)F)nc2cc(NC(=O)C=C)ccc21] | N | O |
| 340 | N | N | N | 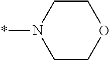 [SMILES: *N1CCOCC1] | 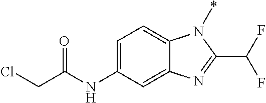 [SMILES: *n1c(C(F)F)nc2cc(NC(=O)CCl)ccc21] | N | O |
| 341 | N | N | N | 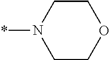 [SMILES: *N1CCOCC1] | 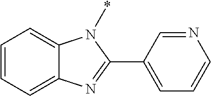 [SMILES: *n1c(-c2cccnc2)nc2ccccc21] | N | O |
| 342 | N | N | N | 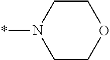 [SMILES: *N1CCOCC1] | 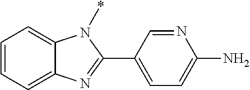 [SMILES: *n1c(-c2ccc(N)nc2)nc2ccccc21] | N | O |
| 343 | N | N | N | 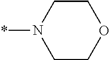 [SMILES: *N1CCOCC1] | 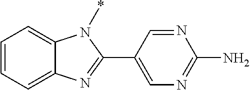 [SMILES: *n1c(-c2cnc(N)nc2)nc2ccccc21] | N | O |
| 344 | N | N | N | 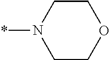 [SMILES: *N1CCOCC1] | 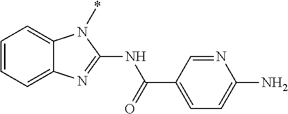 [SMILES: *n1c(NC(=O)c2ccc(N)nc2)nc2ccccc21] | N | O |
| 345 | N | N | N | 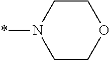 [SMILES: *N1CCOCC1] | 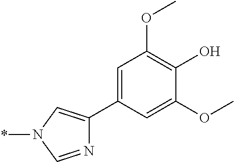 [SMILES: *n1cnc(-c2cc(OC)c(O)c(OC)c2)c1] | N | O |
| 346 | N | N | N | 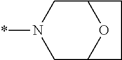 [SMILES: *N1CC2CCC(C1)O2] | 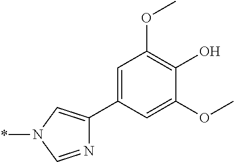 [SMILES: *n1cnc(-c2cc(OC)c(O)c(OC)c2)c1] | N | O |
| 347 | CH | N | N | 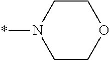 [SMILES: *N1CCOCC1] | 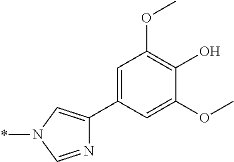 [SMILES: *n1cnc(-c2cc(OC)c(O)c(OC)c2)c1] | N | O |
| 348 | N | CH | N | 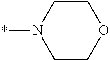 [SMILES: *N1CCOCC1] | 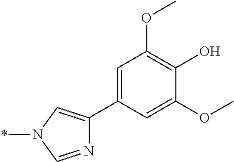 [SMILES: *n1cnc(-c2cc(OC)c(O)c(OC)c2)c1] | N | O |
| 349 | N | N | CH | 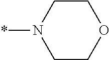 [SMILES: *N1CCOCC1] | 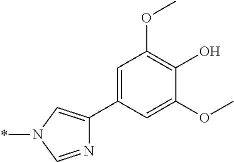 [SMILES: *n1cnc(-c2cc(OC)c(O)c(OC)c2)c1] | N | O |
| 350 | N | N | N | 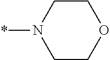 [SMILES: *N1CCOCC1] | 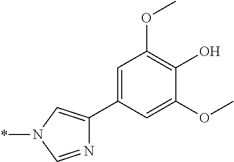 [SMILES: *n1cnc(-c2cc(OC)c(O)c(OC)c2)c1] | N | NH |
| 351 | N | N | N | 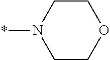 [SMILES: *N1CCOCC1] | 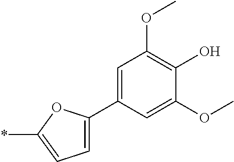 [SMILES: *c1ccc(-c2cc(OC)c(O)c(OC)c2)o1] | N | O |
| 352 | N | N | N | 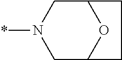 [SMILES: *N1CC2CCC(C1)O2] | 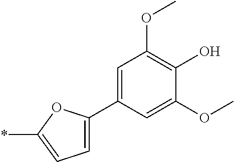 [SMILES: *c1ccc(-c2cc(OC)c(O)c(OC)c2)o1] | N | O |
| 353 | CH | N | N | 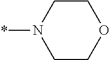 [SMILES: *N1CCOCC1] | 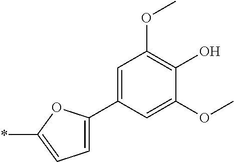 [SMILES: *c1ccc(-c2cc(OC)c(O)c(OC)c2)o1] | N | O |
| 354 | CH | N | N | 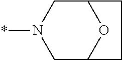 [SMILES: *N1CC2CCC(C1)O2] | 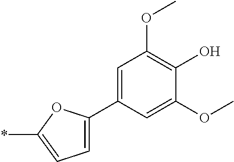 [SMILES: *c1ccc(-c2cc(OC)c(O)c(OC)c2)o1] | N | O |
| 355 | N | CH | N | 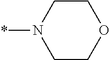 [SMILES: *N1CCOCC1] | 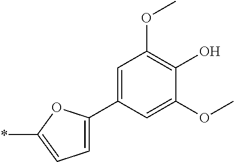 [SMILES: *c1ccc(-c2cc(OC)c(O)c(OC)c2)o1] | N | O |
| 356 | N | CH | N | 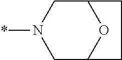 [SMILES: *N1CC2CCC(C1)O2] | 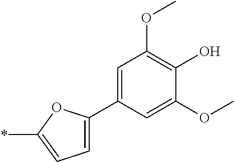 [SMILES: *c1ccc(-c2cc(OC)c(O)c(OC)c2)o1] | N | O |
| 357 | N | N | CH | 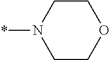 [SMILES: *N1CCOCC1] | 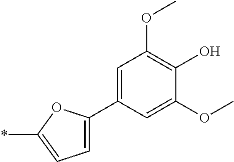 [SMILES: *c1ccc(-c2cc(OC)c(O)c(OC)c2)o1] | N | O |
| 358 | N | N | CH | 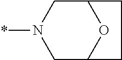 [SMILES: *N1CC2CCC(C1)O2] | 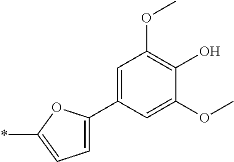 [SMILES: *c1ccc(-c2cc(OC)c(O)c(OC)c2)o1] | N | O |
| 359 | N | N | N | 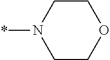 [SMILES: *N1CCOCC1] | 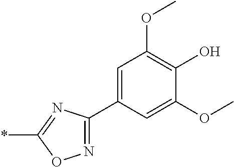 [SMILES: *c1nc(-c2cc(OC)c(O)c(OC)c2)no1] | N | O |
| 360 | N | N | N | 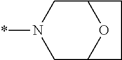 [SMILES: *N1CC2CCC(C1)O2] | 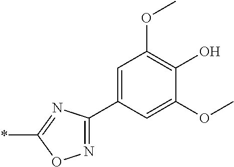 [SMILES: *c1nc(-c2cc(OC)c(O)c(OC)c2)no1] | N | O |
| 361 | N | N | N | 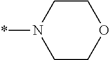 [SMILES: *N1CCOCC1] | 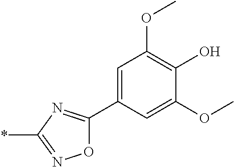 [SMILES: *c1noc(-c2cc(OC)c(O)c(OC)c2)n1] | N | O |
| 362 | N | N | N | 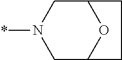 [SMILES: *N1CC2CCC(C1)O2] | 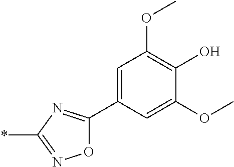 [SMILES: *c1noc(-c2cc(OC)c(O)c(OC)c2)n1] | N | O |
| 363 | N | N | N | 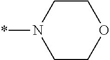 [SMILES: *N1CCOCC1] | 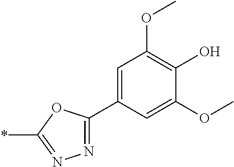 [SMILES: *c1nnc(-c2cc(OC)c(O)c(OC)c2)o1] | N | O |
| 364 | N | N | N | 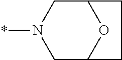 [SMILES: *N1CC2CCC(C1)O2] | 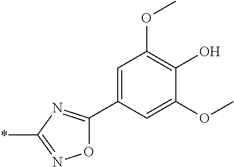 [SMILES: *c1noc(-c2cc(OC)c(O)c(OC)c2)n1] | N | O |
| 365 | N | N | N | 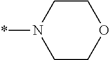 [SMILES: *N1CCOCC1] | 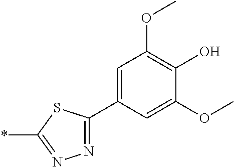 [SMILES: *c1nnc(-c2cc(OC)c(O)c(OC)c2)s1] | N | O |
| 366 | N | N | N | 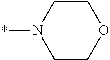 [SMILES: *N1CCOCC1] | 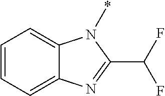 [SMILES: *n1c(C(F)F)nc2ccccc21] | N | 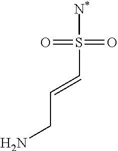 |
| 367 | N | N | N | 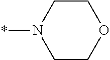 [SMILES: *N1CCOCC1] | 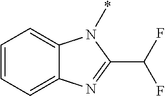 [SMILES: *n1c(C(F)F)nc2ccccc21] | N | 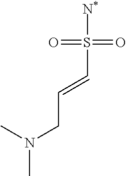 |
| 368 | N | N | N | 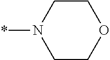 [SMILES: *N1CCOCC1] | 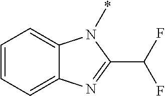 [SMILES: *n1c(C(F)F)nc2ccccc21] | N | 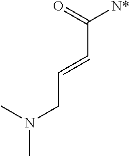 |
| 369 | N | N | N | 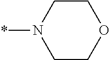 [SMILES: *N1CCOCC1] | 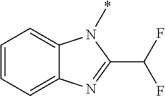 [SMILES: *n1c(C(F)F)nc2ccccc21] | N | 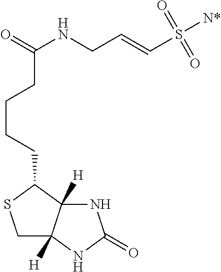 |
| 370 | N | N | N | 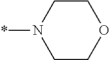 [SMILES: *N1CCOCC1] | 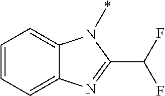 [SMILES: *n1c(C(F)F)nc2ccccc21] | N | 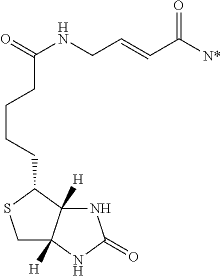 |
| 371 | N | N | N | 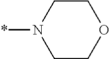 [SMILES: *N1CCOCC1] | 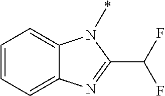 [SMILES: *n1c(C(F)F)nc2ccccc21] | N | 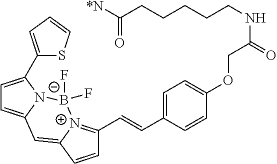 |

TABLE 2
Compounds of formula (II)

| Nr. | Q | U | R¹ | R³ | E¹ | X¹ |
|---|---|---|---|---|---|---|
| 372 | N | N | 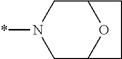 [SMILES: *N1CC2CCC(C1)O2] | 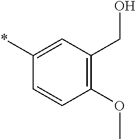 [SMILES: *c1ccc(OC)c(CO)c1] | N | O |
| 373 | N | N | 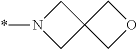 [SMILES: *N1CC2(COC2)C1] | 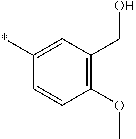 [SMILES: *c1ccc(OC)c(CO)c1] | N | O |
| 374 | N | N | 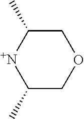 | 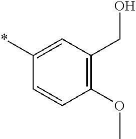 [SMILES: *c1ccc(OC)c(CO)c1] | N | O |
| 375 | N | N | 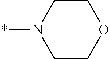 [SMILES: *N1CCOCC1] | 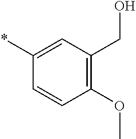 [SMILES: *c1ccc(OC)c(CO)c1] | N | O |
| 376 | N | N | 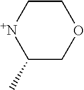 | 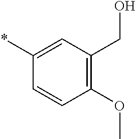 [SMILES: *c1ccc(OC)c(CO)c1] | N | O |

TABLE 3
Compounds of formula (III)

| Nr. | Q | G | R¹ | R³ | E¹ | X¹ |
|---|---|---|---|---|---|---|
| 377 | N | N | 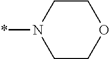 [SMILES: *N1CCOCC1] | 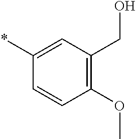 [SMILES: *c1ccc(OC)c(CO)c1] | N | O |

### EXAMPLES

The chemical reactions described in the Examples may be readily adapted to prepare a number of other lipid kinase inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

Abbreviations: hours (h), minutes (min), room temperature (RT), dichloromethane (DCM), dimethylformamide (DMF), ethyl acetate (EtOAc), methanol (MeOH), tetrahydro-furan (THF). Reagents were purchased from commercial suppliers such as Aldrich Chemical Company, Fluorochem, Acros, Lancaster, TCI or Maybridge, and were used without further purification unless otherwise indicated. The reactions set forth below were done generally under a positive pressure of nitrogen or argon or with a drying tube in anhydrous solvents, and the reaction flasks were typically fitted with rubber septa for the introduction of substrates and reagents via syringe. Glassware was oven dried and/or heat dried. Column chromatography was conducted by using Merck silica gel. ¹H NMR spectra were recorded on a Bruker instrument operating at 400 MHz, 500 MHz and 600 MHz. ¹H NMR spectra were obtained in deuterated CDCl₃, d₆-DMSO, CH₃OD or d₆-acetone solutions (reported in ppm), using CHCl₃ as the reference standard (7.25 ppm) or TMS (0 ppm). When peak multiplicities are reported, the following abbreviations are used: (singlet), d (doublet), t (triplet), m (multiplet), br (broadened), dd (doublet of doublets), dt (doublet of triplets). Coupling constants, when given, are reported in Hertz (Hz).

### General Procedure A-1: Triazine/pyrimidine substitution

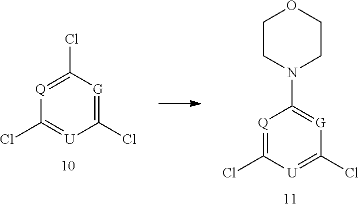

Starting material 10 (2,4,6-trichloro-1,3,5-triazine or 2,4,6-trichloropyrimidine, 1.0 eq.) is suspended in DCM. Morpholine (1.0 eq.) is slowly added during 20 min at −50° C. The reaction mixture is stirred at −50° C. for 25 min before it is poured on water (60 mL). The layers are separated and the water layer is washed twice with DCM and with EtOAc. The combined organic layers are treated with MgSO₄, filtered and dried. Purification by silica gel flash column chromatography (gradient 0% to 50% EtOAc/hexane) yields the desired compounds with the general formula 11.

### General Procedure A-2: Substitution with 2-oxa-azaspiro[3.3.]heptanes

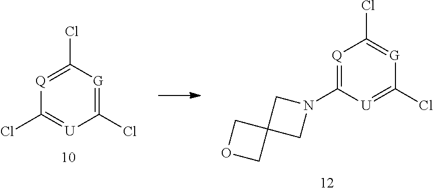

Under nitrogen atmosphere an oven-dried round-bottom flask is charged with sodium hydride (60% dispersion in mineral oil, 2.0 eq.) in dry THF. The solution is cooled down to 0° C. and 2-oxa-azaspiro[3.3.]heptane (1.0 eq.) is added. The reaction mixture is stirred for 30 min at 0° C. Then 10 (1.0 eq.) is added as a solid and the reaction mixture is allowed to reach RT and stirred overnight. The reaction mixture is quenched with H₂O and extracted three times with EtOAc. The combined organic phases are dried over MgSO₄, filtered and the solvent is removed under reduced pressure. Purification by flash column chromatography (2% MeOH/DCM) yields the desired compounds of general formula 12.

### General Procedure B: Suzuki Coupling

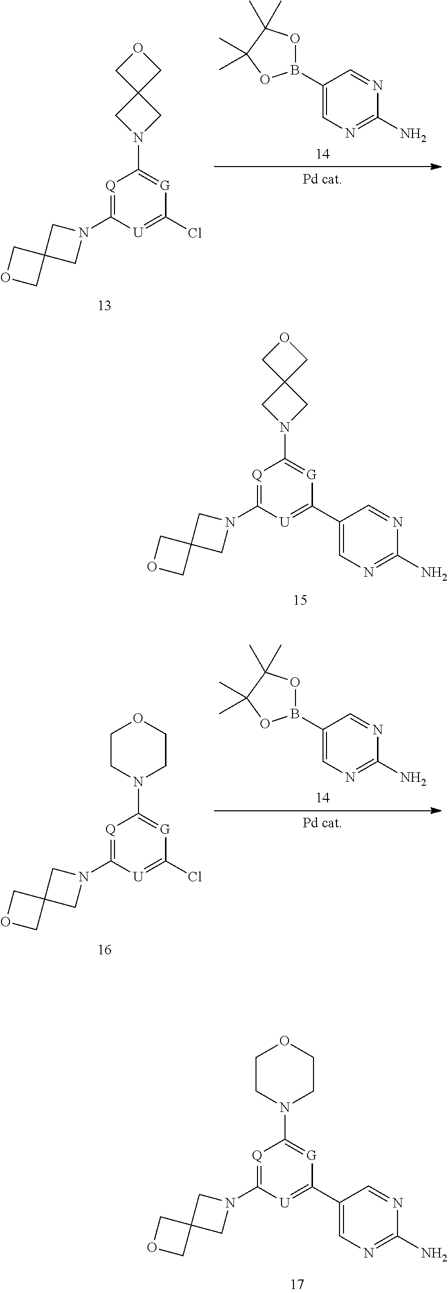

The Suzuki-type coupling reaction is useful to attach a heteroaryl substituent at the 6-position of the triazine or pyridine ring, or at the 4- or 6-position of the pyrimidine ring. Generally, intermediates 13 and 16 are combined with boronic acid pinacol ester 14 (4.0 eq.) in 1,2-dimethoxyethane and 2 M Na₂CO₃ (3:1) for 15 min. A catalytic amount of a palladium reagent dichloro-1,1′-bis(diphenylphosphino)ferrocene palladium (II) (0.025 eq.) is added and the high pressure glass vessel containing the mixture is bubbled with argon gas and sealed. The reaction mixture is then heated at 90° C. for 15 h or more, cooled down and diluted with EtOAc. The organic solution is washed with a mixture of water: Na₂CO₃ (sat.): NH₄OH(NH₄OH conc. 32% in water)=5:4:1, NH₄Cl (sat.) and brine, dried over MgSO₄, filtered and concentrated. The residue is purified by silica gel flash column chromatography or if necessary by reverse phase HPLC.

### Example P1

### 6-(4-(2-(Difluoromethyl)-1H-benzo[d]imidazol-1-yl)-6-(4-methylpiperazin-1-yl)-1,3,5-triazin-2-yl)-2-oxa-6-azaspiro[3.3]heptane (306)

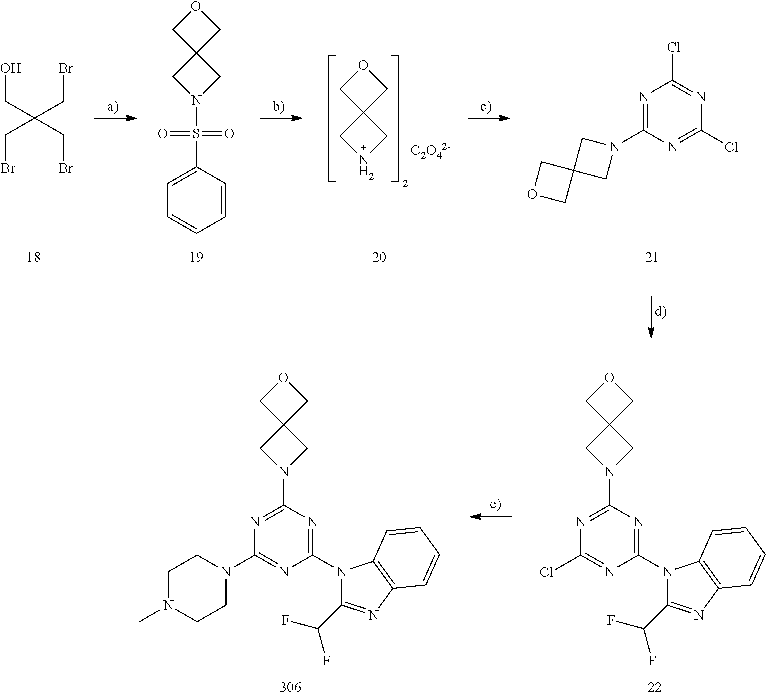

### Step a) and b) were Accomplished According to Procedures of Georg, W., et al., Angew. Chem. Int. Ed. 47:4512-4515 (2008)

### Step c): 6-(4,6-dichloro-1,3,5-triazin-2-yl)-2-oxa-6-azaspiro[3.3]heptane (21)

Following the general procedure A-2,2-oxa-azaspiro[3.3.]heptane (50.0 mg, 173 μmol, 1.0 eq.) is deprotoanated with sodium hydride and reacted with cyanuric chloride (32.0 mg, 173 μmol, 1.0 eq.) to give the title compound as a white solid (37.0 mg, 86%). R_{F}: 0.85 (DCM/MeOH, 9:1 v/v); ¹H NMR (CDCl₃, 400 MHz): δ 4.83 (s, 4H), 4.39 (s, 4H). ¹³C NMR (100 MHz, CDCl₃): δ 170.4, 163.9, 80.5, 59.6, 39.0; EI-MS (70 eV, C₈H₈Cl₂N₄O): Calc'd. 247.02 (M⁺). Found 248.00.

### Step d): 6-(4-chloro-6-(2-(difluoromethyl)-1H-benzo[d]imidazol-1-yl)-1,3,5-triazin-2-yl)-2-oxa-6-azaspiro[3.3]heptane (22)

Under nitrogen atmosphere an oven-dried round-bottom flask was charged with compound 21 (73.0 mg, 295 μmol, 1.0 eq.) in dry DMF (2 mL). The solution was cooled down to 0° C. and potassium carbonate (59.1 mg, 425 μmol, 1.4 eq.) and 2-(difluoro-methyl)-1H-benzoimidazole (69.5 mg, 414 μmol, 1.4 eq.) were added. The reaction mixture was stirred for 30 min at 0° C. and then for 2 h at RT. The solvent was removed under high vacuum and the remaining residue was purified directly by flash column chromatography (1% MeOH/DCM) to yield the title compound as a white solid (53.7 mg, 48%). R_{F}: 0.48 (DCM/MeOH, 95:5 v/v); ¹H NMR (CDCl₃, 400 MHz): δ 8.48 (d, J=7.6 Hz, 1H), 7.90 (d, J=7.2 Hz, 1H), 7.64 (t, J=53.6 Hz, 1H), 7.46-7.44 (m, 2H), 4.90 (s, 4H), 4.49 (d, J=9.6 Hz, 4H).

### Step e): 6-(4-(2-(difluoromethyl)-1H-benzo[d]imidazol-1-yl)-6-(4-methyl-piperazin-1-yl)-1,3,5-triazin-2-yl)-2-oxa-6-azaspiro[3.3]heptane (306)

Under nitrogen atmosphere an oven-dried round-bottom flask was charged with compound 22 (35.0 mg, 92.4 μmol, 1.0 eq.) in dry DMF (3 mL). Potassium carbonate (40.9 mg, 296 μmol, 3.2 eq.) and 1-methylpiperazine (12.3 μL, 111 μmol, 1.2 eq.) were added and the resulting reaction mixture was stirred for 2 hours at room temperature. The solvent was removed under high vacuum and the remaining residue was purified directly by flash column chromatography (2% MeOH/DCM) to yield the title compound as a white solid (39.4 mg, 96%). R_{F}: 0.15 (methylene chloride/methanol, 95:5 v/v); ¹H-NMR (DMSO, 400 MHz): δ 8.43 (d, J=8.0 Hz, 1H), 7.84 (d, J=8.0 Hz, 1H), 7.77 (t, J=52.8 Hz, 1H), 7.50 (t, J=7.6 Hz, 1H), 7.44 (td, J=7.6, 0.8 Hz, 1H), 4.74 (d, J=5.2 Hz, 4H), 4.33 (d, J=35.6 Hz), 3.79 (t, J=4.2 Hz, 4H), 2.39 (sbr, 4H), 2.22 (s, 3H); ESI-MS (C₂₁H₂₄F₂N₈O): Calc'd. 443.21 (M⁺). Found 443.3.

### Example P2

### 6-(4-(2-(difluoromethyl)-1H-benzo[d]imidazol-1-yl)-6-morpholino-1,3,5-triazin-2-yl)-2-oxa-6-azaspiro[3.3]heptanes (299)

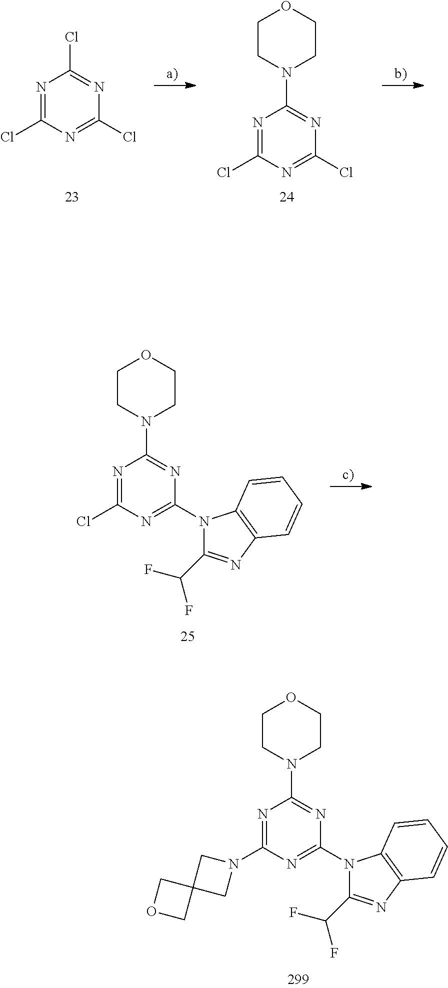 [SMILES: Clc1nc(Cl)nc(Cl)n1 ; Clc1nc(Cl)nc(N2CCOCC2)n1 ; FC(F)c1nc2ccccc2n1-c1nc(Cl)nc(N2CCOCC2)n1 ; FC(F)c1nc2ccccc2n1-c1nc(N2CCOCC2)nc(N2CC3(COC3)C2)n1]

### Step a): 4-(4,6-dichloro-1,3,5-triazin-2-yl)morpholine (24)

Following the general procedure A-1, cyanuric chloride (10.0 g, 54.2 mmol, 1.0 eq.) is reacted with morpholine (4.70 ml, 54.2 mmol, 1.0 eq.). The reaction mixture was purified by silica gel flash column chromatography (70% hexane/ethyl acetate) to yield the title compound as a colourless solid (3.60 g, 28%). R_{F}: 0.72 (hexane/EtOAc 1:1 v/v); ¹H NMR (CDCl₃, 400 MHz) δ 3.88 (t, J=4.9 Hz, 4H), 3.75 (t, J=4.8 Hz, 4H); ¹³H NMR (CDCl₃, 100 MHz) δ 170.85, 164.50, 66.79, 44.87; ESI-MS (C₇H₈Cl₂N₄O): Calc'd. 258.0 (M+Na)⁺. Found 258.6.

### Step b): 4-(4-chloro-6-(2-(difluoromethyl)-1H-benzo[d]imidazol-1-yl)-1,3,5-triazin-2-yl)morpholine (25)

Compound 24 (425 μmol, 1.0 eq.) was dissolved in DMF (2 mL) and cooled to −5° C., treated with anhydrous potassium carbonate (1.44 eq.) and 2-(difluoromethyl)-1H-benzo[d]imidazole (1.4 eq.), stirred for 30 min and further stirred at RT for 4 h. The reaction mixture was diluted with water and the precipitate was filtered and washed with small amounts of water. Purification was done by silica gel flash column chromatography.

### Step c): 6-(4-(2-(difluoromethyl)-1H-benzo[d]imidazol-1-yl)-6-morpholino-1,3,5-triazin-2-yl)-2-oxa-6-azaspiro[3.3]heptane (299)

Under nitrogen atmosphere an oven-dried round-bottom flask was charged with compound 25 (50.0 mg, 136 μmol, 1.0 eq.) in dry DMF (3 mL). Potassium carbonate (60.3 mg, 436 μmol, 3.20 eq.) and 2-oxa-azaspiro[3.3.]heptane (23.6 mg, 81.8 μmol, 0.6 eq.) were added and the resulting reaction mixture was stirred for 3 h at RT. The solvent was removed under high vacuum and the remaining residue was purified directly by flash column chromatography (1% MeOH/DCM) to yield the title compound as a white solid (41.4 mg, 71%). R_{F}: 0.21 (DCM/MeOH, 95:5 v/v); ¹H NMR (CDCl₃, 400 MHz): δ 8.40 (d, J=7.6 Hz, 1H), 7.87 (d, J=7.2 Hz, 1H), 7.63 (t, J=53.6 Hz, 1H), 7.43-7.37 (m, 2H), 4.85 (s, 4H), 4.32 (d, J=24.4 Hz, 4H), 3.85 (t, J=4.4 Hz, 4H), 3.78-3.76 (m, 4H); ¹³C NMR (100 MHz, CDCl₃): δ 165.3, 164.9, 162.0, 142.1, 133.8, 126.0, 124.6, 121.4, 116.5, 108.7, 81.0, 66.8, 59.1, 44.1, 39.1; ESI-MS (C₂₀H₂₁F₂N₇O₂): Calc'd. 430.17 (M⁺). Found 430.10.

### Example P3

### 6-(4-(2-(difluoromethyl)-1H-benzo[d]imidazol-1-yl)-6-(6-oxa-2-aza-spiro[3.3]heptan-2-yl)-1,3,5-triazin-2-yl)-2-oxa-6-azaspiro-[3.3]heptane (26)

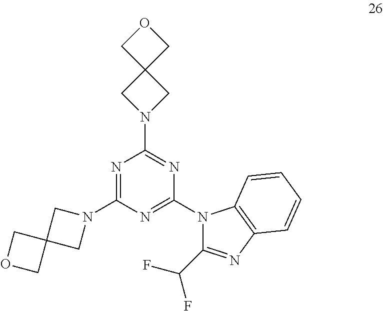 [SMILES: FC(F)c1nc2ccccc2n1-c1nc(N2CC3(COC3)C2)nc(N2CC3(COC3)C2)n1]

Under nitrogen atmosphere an oven-dried round-bottom flask was charged with 6-(4-chloro-6-(2-(difluoromethyl)-1H-benzo[d]imidazol-1-yl)-1,3,5-triazin-2-yl)-2-oxa-6-azaspiro-[3.3]heptane 22 (47.0 mg, 124 μmol, 1.0 eq.) in dry DMF (3 mL). Potassium carbonate (54.9 mg, 397 μmol, 3.2 eq.) and 2-oxa-azaspiro[3.3.]heptane (21.5 mg, 74.5 μmol, 0.6 eq.) were added and the resulting reaction mixture was stirred for 4 h at RT. The solvent was removed under high vacuum and the remaining residue was purified directly by flash column chromatography (2% MeOH/DCM) to yield the title compound as a white solid (49.3 mg, 90%). R_{F}: 0.22 (DCM/MeOH 95:5 v/v); ¹H NMR (CDCl₃, 400 MHz): δ 8.49 (d, J=8.0 Hz, 1H), 7.88-7.84 (m, 1H), 7.72-7.36 (m, 3H), 4.86 (s, 8H), 4.33 (d, J=20.0 Hz, 8H); ¹³C NMR (CDCl₃, 100 MHz,): δ 165.4, 161.9, 146.8, 142.3, 134.0, 126.2, 124.9, 121.6, 117.1, 109.0, 81.2, 59.4, 39.3; ESI-MS (C₂₁H₂₁F₂N₇O₂): Calc'd. 480.14 (M+K)⁺. Found 480.20.

### Example P4

### 6-(2-(2-(difluoromethyl)-1H-benzo[d]imidazol-1-yl)-6-morpholino-pyrimidin-4-yl)-2-oxa-6-azaspiro[3.3]heptane (300)

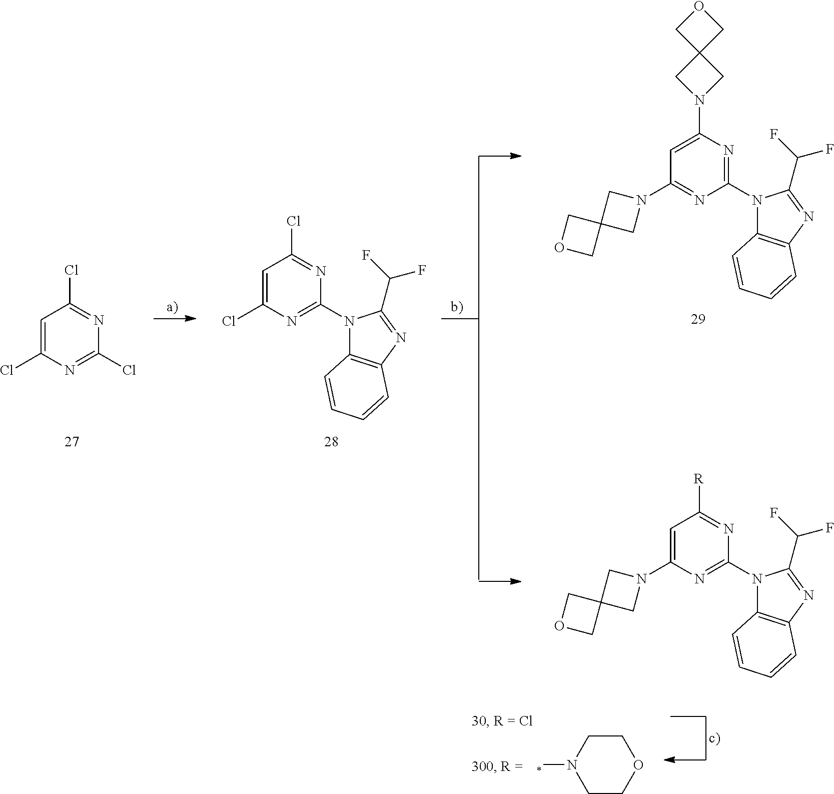

### Step a): 1-(4,6-dichloropyrimidin-2-yl)-2-(difluoromethyl)-1H-benzo[d]imidazole (28)

Under nitrogen atmosphere an oven-dried round-bottom flask was charged with 2,4,6-trichloropyrimidine (31.0 μL, 273 μmol, 1.0 eq.) in dry DMF (2 mL). The solution was cooled down to −5° C. and potassium carbonate (65.6 mg, 474 μmol, 1.74 eq.) and 2-difluoromethyl-1H-benzimidazole (41.3 mg, 245 μmol, 0.9 eq.) were added. The reaction mixture was stirred for 30 min at −5° C. and then for 18 h at RT. The solvent was removed under high vacuum and the remaining residue was purified directly by flash column chromatography (20% EtOAc/hexane) to yield the title compound as a white solid (60.0 mg, 70%). R_{F}: 0.44 (hexane/EtOAc, 4:1 v/v); ¹H-NMR (CDCl₃, 400 MHz): δ 8.50 (d, J=8.0 Hz, 1H), 7.93 (d, J=8.4 Hz, 1H), 7.69 (t, J=53.6 Hz, 1H), 7.55-7.45 (m, 2H), 7.37 (s, 1H); ¹⁹F-NMR (CDCl₃, 400 MHz): δ −119.1 (d, J=56.8 Hz, 2F); ESI-MS (C₁₂H₆Cl₂F₂N₄): Calc'd. 314.99 (M⁺). Found 315.90.

### Step b): 6-(2-(2-(difluoromethyl)-1H-benzo[d]imidazol-1-yl)-6-(6-oxa-2-azaspiro-[3.3]heptan-2-yl)pyrimidin-4-yl)-2-oxa-6-azaspiro[3.3]-heptane (29)

Under nitrogen atmosphere an oven-dried round-bottom flask was charged with compound 28 (56.0 mg, 178 μmol, 1.0 eq.) in dry DMF (2 mL). The solution was cooled down to −5° C. and potassium carbonate (68.8 mg, 498 μmol, 2.80 eq.) and 2-oxa-6-azaspiro[3.3.]heptane (25.6 mg, 88.9 μmol, 1.0 eq.) were added. The reaction mixture was stirred for 30 min at −5° C. and then for 18 h at RT. The solvent was removed under high vacuum and the remaining residue was purified directly by flash column chromatography (gradient from 0% to 100% EtOAc/hexane) to yield the mono-substituted pyrimidine derivative 30 as a white solid (27.3 mg, 41%) and di-substituted pyrimidine derivative 29 as a white solid (14.9 mg, 19%). Compound 30: R_{F}: 0.38 (hexane/EtOAc 1:2 v/v); ¹H-NMR (CDCl₃, 400 MHz): δ 8.47 (d, J=7.6 Hz, 1H), 7.89 (d, J=7.6 Hz, 1H), 7.68 (t, J=53.6 Hz, 1H), 7.48-7.34 (m, 2H), 6.11 (s, 1H), 4.89 (s, 4H), 4.36 (sbr, 4H); ¹⁹F-NMR (CDCl₃, 400 MHz): δ −118.3 (d, J=57.2 Hz, 2F); ESI-MS (C₁₇H₁₄ClF₂N₅O): Calc'd. 378.09 (M⁺). Found 378.20. Compound 29: R_{F}: 0.06 (hexane/EtOAc 1:2 v/v); ¹H-NMR (CDCl₃, 400 MHz): δ 8.49 (d, J=8.0 Hz, 1H), 7.87 (d, J=8.0 Hz, 1H), 7.72 (t, J=54.0 Hz, 1H), 7.43-7.35 (m, 2H), 4.86 (s, 8H), 4.81 (s, 1H), 4.23 (s, 8H); ¹⁹F-NMR (CDCl₃, 400 MHz): δ −117.9 (d, J=57.2 Hz, 2F); ESI-MS (C₂₂H₂₂F₂N₆O₂): Calc'd. 441.18 (M⁺). Found 441.30.

### Step c): 6-(2-(2-(difluoromethyl)-1H-benzo[d]imidazol-1-yl)-6-morpholino-pyrimidin-4-yl)-2-oxa-6-azaspiro[3.3]heptane (300)

Under nitrogen atmosphere an oven-dried round-bottom flask was charged with mono-substituted pyrimidine derivative 30 (10.0 mg, 26.5 μmol, 1.0 eq.) dissolved in an excess of morpholine (300 μL, 3.47 mmol, 130 eq.). The reaction mixture was heated to 80° C. and stirred at this temperature for 2 h. The reaction mixture was allowed to reach RT, poured into water (5 mL) and extracted with ethyl acetate (5 mL). The organic phase was washed with brine (5 mL), dried over MgSO₄, filtered and the solvent was removed under reduced pressure. The residue was purified by flash column chromatography (gradient from 50% to 100% EtOAc/hexane) to yield the title compound as a white solid (7.30 mg, 45%). R_{F}: 0.22 (hexane/EtOAc 1:1 v/v); ¹H-NMR (CDCl₃, 400 MHz): δ 8.34 (d, J=8.0 Hz, 1H), 7.88 (d, J=8.0 Hz, 1H), 7.63 (t, J=53.6 Hz, 1H), 7.43-7.36 (m, 2H), 5.16 (s, 1H), 4.88 (s, 4H), 4.28 (s, 4H), 3.83 (t, J=4.8 Hz, 4H), 3.61 (t, J=4.8 Hz, 4H); ¹⁹F-NMR (CDCl₃, 400 MHz): δ −117.6 (d, J=57.2 Hz, 2F). ESI-MS (C₂₁H₂₂F₂N₆O₂): Calc'd. 467.28 (M+K)⁺. Found 467.20.

### Example P5

### Tert-butyl-6-(4-(2-(difluoromethyl)-1H-benzo[d]imidazol-1-yl)-6-morpholino-1,3,5-triazin-2-yl)-2,6-diazaspiro[3.3]heptane-2-carboxylate (31)

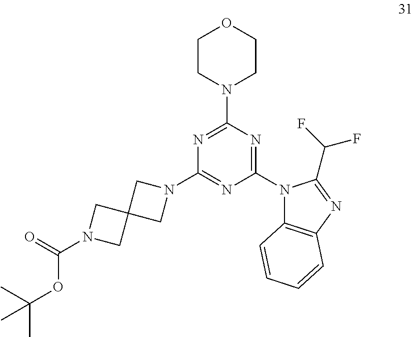 [SMILES: CC(C)(C)OC(=O)N1CC2(C1)CN(c1nc(N3CCOCC3)nc(-n3c(C(F)F)nc4ccccc43)n1)C2]

4-(4-Chloro-6-(2-(difluoromethyl)-1H-benzo[d]imidazol-1-yl)-1,3,5-triazin-2-yl)morpholine 25 (30.0 mg, 81.8 μmol, 1.0 eq.) was dissolved in DMF (3 mL). Potassium carbonate (36.2 mg, 262 μmol, 3.2 eq.) and tert-butyl 2,6-diazaspiro-[3.3]heptane-2-carboxylate (23.6 mg, 49.1 μmol, 0.6 eq.) were added and the resulting reaction mixture was stirred for 3 h at RT. The solvent was removed under high vacuum and the remaining residue was purified directly by flash column chromatography (SiO₂, gradient 0% to 1% MeOH in DCM) to provide the title compound as a colourless solid (41.0 mg, 77.6 μmol, 95%). R_{F}: 0.44 (DCM/MeOH 95:5 v/v); ¹H NMR (CDCl₃, 400 MHz): δ 8.39 (d, J=8.0 Hz, 1H), 7.86 (d, J=7.2 Hz, 1H), 7.62 (t, J=54.0 Hz, 1H), 7.39-7.37 (m, 2H), 4.28 (d, J=32.0 Hz, 4H), 4.13 (s, 4H); 3.85 (t, J=4.4 Hz, 4H), 3.76 (sbr, 4H), 1.44 (s, 9H); ¹⁹F-NMR (CDCl₃, 376 MHz): δ −117.9 (d, J=53.4 Hz, 2F); ESI-MS (C₂₅H₃₀F₂N₈O₃): Calc'd. 551.23 [M+Na]⁺. Found 551.30.

### Example P6

### 4-(4-(2-(difluoromethyl)-1H-benzo[d]imidazol-1-yl)-6-(2,6-diaza-spiro[3.3]heptan-2-yl)-1,3,5-triazin-2-yl)morpholine (303)

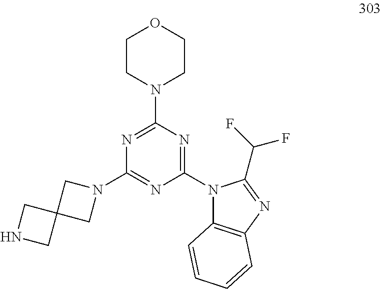 [SMILES: FC(F)c1nc2ccccc2n1-c1nc(N2CCOCC2)nc(N2CC3(CNC3)C2)n1]

Tert-Butyl-6-(4-(2-(difluoromethyl)-1H-benzo[d]imidazol-1-yl)-6-morpholino-1,3,5-triazin-2-yl)-2,6-diazaspiro[3.3]heptane-2-carboxylate 33 (40.0 mg, 75.7 μmol, 1.0 eq.) was dissolved in a mixture of DCM (0.6 mL) and trifluoroacetic acid (0.3 mL). The reaction mixture was stirred for 2 h at RT. Then the solvent was removed under reduced pressure. The residue was dissolved in ethyl acetate (5 mL) and washed twice with saturated NaHCO₃ solution (2×5 mL). The organic phase was dried over MgSO₄, filtered and the solvent was removed under reduced pressure to provide the title compound as a brownish solid (25.9 mg, 80%). ¹H NMR (CDCl₃, 400 MHz): δ 8.44 (d, J=7.6 Hz, 1H), 7.86 (d, J=7.2 Hz, 1H), 7.79 (t, J=53.0 Hz, 1H), 7.51-7.42 (m, 2H), 4.35 (d, J=42.4 Hz, 4H), 4.20 (s, 4H); 3.80 (sbr, 4H), 3.69 (sbr, 4H); ¹⁹F-NMR (CDCl₃, 376 MHz): δ −116.4 (d, J=53.0 Hz, 2F); ESI-MS (C₂₀H₂₂F₂N₈O): Calc'd. 429.19 [M+H]⁺. Found 429.20.

### Example P7

### 1-(6-(4-(2-(difluoromethyl)-1H-benzo[d]imidazol-1-yl)-6 morpholino-1,3,5-triazin-2-yl)-2,6-diazaspiro[3.3]heptan-2-yl)prop-2-en-1-one (317)

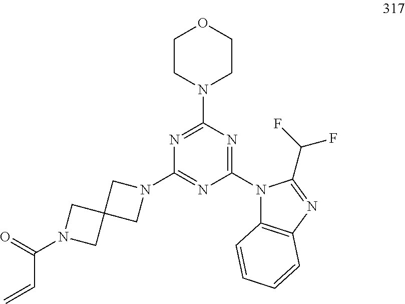 [SMILES: C=CC(=O)N1CC2(C1)CN(c1nc(N3CCOCC3)nc(-n3c(C(F)F)nc4ccccc43)n1)C2]

Compound 303 (20 mg, 46.7 μmol, 1.0 eq.) was dissolved in DCM (2 mL). Diisopropylethylamine (8.7 μL, 51.3 μmol, 1.1 eq.) and acrylic anhydride (5.4 μL, 46.7 μmol, 1.0 eq.) were added and the reaction mixture was stirred for 1.5 h at RT. The solvent was removed under reduced pressure and the residue was purified by flash column chromatography (SiO₂, gradient 0% to 2% MeOH in DCM) to provide the title compound as a colorless solid (15.3 mg, 68%). R_{F}: 0.60 (DCM/MeOH 95:5 v/v); ¹H NMR (CDCl₃, 400 MHz): δ 8.41 (dd, J=7.2, 1.6 Hz, 1H), 7.88 (dd, J=7.2, 1.6 Hz, 1H), 7.63 (t, J=53.6 Hz, 1H), 7.41-7.37 (m, 2H), 6.37 (dd, J=17.0, 1.6 Hz, 1H), 6.18 (dd, J=17.0, 10.4 Hz, 1H), 5.72 (dd, J=10.4, 1.6 Hz, 1H), 4.42 (d, J=12.8 Hz, 4H), 4.29 (sbr, 4H); 3.87 (sbr, 4H), 3.78 (sbr, 4H); ¹⁹F-NMR (CDCl₃, 376 MHz): δ −116.7 (d, J=53.0 Hz, 2F); ESI-MS (C₂₃H₂₄F₂N₈O₂): Calc'd. 505.19 [M+H]⁺. Found 505.40.

### Example P8

### 6-Morpholino-2-(2-oxa-6-azaspiro[3.3]heptan-6-yl)-[4,5′-bipyrimidin]-2′-amine (254)

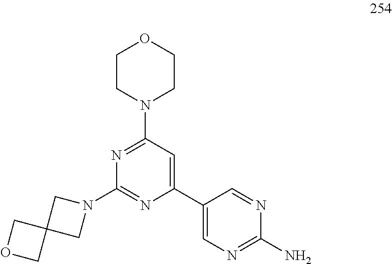 [SMILES: Nc1ncc(-c2cc(N3CCOCC3)nc(N3CC4(COC4)C3)n2)cn1]

Following the general procedure B, 6-(4-chloro-6-morpholinopyrimidin-2-yl)-2-oxa-6-azaspiro[3.3]heptane (35.0 mg, 118 μmol, 1.0 eq.) was heated with 2-aminopyrimidine-5-boronic acid pinacol ester (104 mg, 472 μmol, 4.0 eq.) for 20 h. The residue was purified with flash column chromatography (SiO₂, gradient 50% to 100% EtOAc in hexane with 1% triethylamine) and provided the title compound as a slightly yellow solid (7.5 mg, 18%). R_{F}: 0.24 (EtOAc/triethylamine 100:1 v/v); ¹H-NMR (400 MHz, CDCl₃): δ 8.86 (s, 2H), 6.15 (s, 1H), 5.25 (sbr, 2H), 4.84 (s, 4H), 4.26 (s, 4H), 3.78 (t, J=5.0 Hz, 4H), 3.63 (t, J=4.8 Hz, 4H); ESI-MS (C₁₇H₂₁N₇O₂): Calc'd. 356.18 [M+H]⁺. Found 356.30.

### Example P9

### 4-Morpholino-6-(2-oxa-6-azaspiro[3.3]heptan-6-yl)-[2,5′-bipyrimidin]-2′-amine (253)

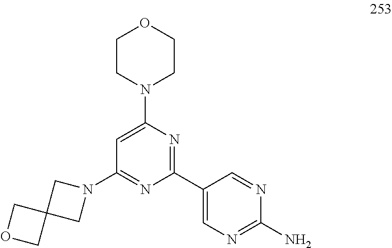 [SMILES: Nc1ncc(-c2nc(N3CCOCC3)cc(N3CC4(COC4)C3)n2)cn1]

Following the general procedure B, 6-(2-chloro-6-morpholinopyrimidin-4-yl)-2-oxa-6-azaspiro[3.3]heptane (35.0 mg, 118 μmol, 1.0 eq.) was heated with 2-aminopyrimidine-5-boronic acid pinacol ester (104 mg, 472 μmol, 4.0 eq.) for 17 h. The residue was purified with flash column chromatography (SiO₂, gradient 50% to 100% EtOAc in hexane with 1% triethylamine) provided the title compound as a beige solid (5.2 mg, 12%). R_{F}: 0.24 (EtOAc/triethylamine 100:1 v/v); ¹H-NMR (400 MHz, CDCl₃): δ 9.18 (s, 2H), 5.21 (sbr, 2H), 5.18 (s, 1H), 4.86 (s, 4H), 4.22 (s, 4H), 3.80 (t, J=4.8 Hz, 4H), 3.60 (t, J=4.8 Hz, 4H); ESI-MS (C₁₇H₂₁N₇O₂): Calc'd. 356.18 [M+H]⁺. Found 356.30.

### Example P10

### 2-Morpholino-6-(2-oxa-6-azaspiro[3.3]heptan-6-yl)-[4,5′-bipyrimidin]-2′-amine (255)

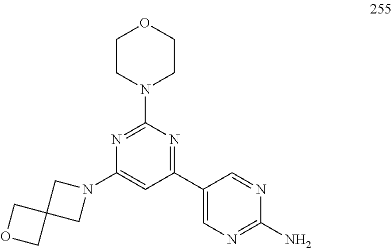 [SMILES: Nc1ncc(-c2cc(N3CC4(COC4)C3)nc(N3CCOCC3)n2)cn1]

Following the general procedure B, 6-(6-chloro-2-morpholinopyrimidin-4-yl)-2-oxa-6-azaspiro[3.3]heptane (35.0 mg, 118 μmol, 1.0 eq.) was heated with 2-aminopyrimidine-5-boronic acid pinacol ester (104 mg, 472 μmol, 4.0 eq.) for 17 h. The residue was purified with flash column chromatography (SiO₂, gradient 50% to 100% EtOAc in hexane with 1% triethylamine) provided the title compound as a beige solid (3.7 mg, 10.4 μmol, 9%). R_{F}: 0.22 (EtOAc/triethylamine 100:1 v/v); ¹H-NMR (400 MHz, CDCl₃): δ 8.86 (s, 2H), 5.84 (s, 1H), 5.23 (sbr, 2H), 4.85 (s, 4H), 4.23 (s, 4H), 3.82 (t, J=4.8 Hz, 4H), 3.76 (t, J=4.8 Hz, 4H); ESI-MS (C₁₇H₂₁N₇O₂): Calc'd. 356.18 [M+H]⁺. Found 356.30.

### Example P11

### In Cell Western-Inhibition Assay

Inhibitor efficacy of compounds of the invention was measured by a cell assay employing the following protocol:

80,000 cells/well were plated in black 96 well view plates (Packard), the homogeneity checked under the microscope, and the cells incubated for 24 h. The medium was discarded and replaced with 100 μl fresh medium. 1 μl of 100× concentrated compound of the invention or DMSO (as control) were added to the medium (each sample as duplicates) and incubated for 3 h at 37° C. 60 μl para-formaldehyde 10% was added to give 4% final concentration, and incubated for 20 min at RT to fix the cells. After washing three times with 200 μl PBS/0.1% Triton/X-100 for 5 min, the plates were blocked with 100 μl 10% goat serum in PBS for 1 h. On a shaker, 50 μl antibodies diluted 1:500 in PBS against pPKB Ser473 (Cell Signalling) and PKB (gift from E. Hirsch, Torino) or pS6 Ser 235/236 (Cell Signalling) were incubated overnight at 4° C. After washing three times with PBS for 5 min, 50 μl secondary antibody anti-rabbit IRDye800 (LI-COR, 1:800) and anti-mouse IRDye680 (LI-COR, 1:500) in PBS were applied at RT in the dark for 1 h. Plates were washed three times with PBS for 5 min and scanned on an Odyssey reader. The more phosphorylated PKB was measured on the Odyssey scan, the higher the pPKB/PKB values were, i.e. the less strong was inhibition of signalling. A summary of the results obtained for some exemplary compounds is depicted in Table 3. Assessment of compound permeability was indirectly intepretated by using this assay. The compounds were applied to the apical surface of cell monolayers and compound permeation into the cellular compartment could be interpretated by measuring the inhibition of PI3Ks.

TABLE 4

| | pPKB/PKB | pPKB/PKB | pS6 | pS6 |
|---|---|---|---|---|
| Example | 1 μM | 10 μM | 1 μM | 10 μM |
| 299 | ++++ | ++++ | +(+) | ++++ |
| 254 | (+) | ++ | (+) | ++ |
| 253 | (+) | +++(+) | − | +++ |
| 255 | (+) | +++(+) | − | +++ |
| 300 | +++ | ++++ | ++ | +++ |
| 303 | ++++ | ++++ | + | +++ |
| 317 | ++(+) | ++++ | ++ | +++ |

1. A compound of formula (I), 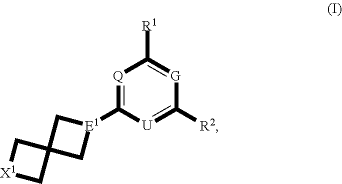
wherein
G is CH or N, Q is CH or N, and U is CH or N, with the proviso that at least two of G, Q and U are N, or one of G and U together with R² forms an anullated pyridine ring further substituted by R³, and the other one of G and U is N and Q is N;
E¹ and E² are, independently of each other, CR⁴, N, N⁺R⁴, or N→O;
X¹ and X² are, independently of each other, CHR⁴, CH₂CH₂, NR⁴, NR⁴→O, or O;
R¹ is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, a linker carrying a reactive group and/or a tag, or 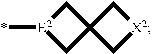
R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;
R³ is optionally substituted amino, optionally substituted C₆-C₂₀-aryl, or optionally substituted C₁-C₁₉-heteroaryl;
R⁴ is hydrogen, C₁-C₆-alkyl, C₁-C₆-acyl, C₁-C₆-acylamino-C₁-C₆-alkyl, a reactive group or a linker carrying a reactive group and/or a tag;
and tautomers, prodrugs, metabolites, solvates and pharmaceutically acceptable salts thereof.

2. The compound of formula (I) according to claim 1, wherein
G is CH or N, Q is CH or N, and U is CH or N, with the proviso that at least two of G, Q and U are N;
E¹ and E² are, independently of each other, CR⁴, N, N⁺R⁴, or N→O;
X¹ and X² are, independently of each other, CHR⁴, CH₂CH₂, NR⁴, NR⁴→O, or O;
R¹ is optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, or 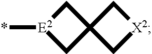
R² is optionally substituted C₆-C₂₀ aryl or optionally substituted C₁-C₂₀ heteroaryl; and
R⁴ is hydrogen, methyl, a reactive group selected from acryloyl, methacryloyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-trichloroacetyl, methylsulfonyloxyacetyl, 2-chloropropionyl, 2,3-epoxypropionyl, (phenylthio)thiocarbonyl, 2-nitro-phenoxycarbonyl, 4-fluorophenoxycarbonyl, and 4-(3-(trifluoromethyl)-3H-diazirin-3-yl)-benzamide, a chain of 1 to 20 optionally substituted methylene groups either directly linked to X¹, X², E¹ or E², or linked to the reactive group, or such chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-trichloroacetyl, methylsulfonyloxyacetyl, 2-chloropropionyl, 2,3-epoxypropionyl, (phenylthio)thiocarbonyl, 2-nitrophenoxycarbonyl, and 4-fluorophenoxycarbonyl;
and tautomers, solvates and pharmaceutically acceptable salts thereof.

3. The compound of formula (I) according to claim 1, wherein
G is CH or N, Q is CH or N, and U is CH or N, with the proviso that at least two of G, Q and U are N;
E¹ and E² are, independently of each other, N or N⁺R⁴;
X¹ and X² are, independently of each other, NR⁴ or O;
R¹ is optionally substituted 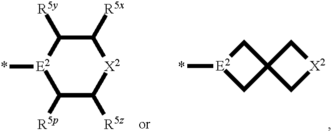
wherein R^{5x}, R^{5y}, R^{5z} and R^{5p} are, independently of each other, hydrogen, halogen, cyano, optionally substituted C₁-C₆ alkyl, C₂-C₆ alkenyl, or C₂-C₆ alkynyl, or one or two of R^{5x}, R^{5y}, R^{5z} and R^{5p} are two geminal substituents methyl and the other ones are hydrogen, or R^{5x} and R^{5y}, or R^{5z} and R^{5p} form together an anullated five- or six-membered carbocyclyl, heterocyclyl, aryl or heteroaryl ring, or R^{5x} and R^{5p} form together bridging ethylene, or R^{5y} and R^{5p} form together bridging ethylene;
R² is phenyl, optionally substituted by one or more groups halogen, C₁-C₆-alkyl, halo-C₁-C₆-alkyl, hydroxy, C₁-C₆-alkoxy, optionally C₁-C₆-alkylated or C₁-C₂₀-acylated amino, or optionally substituted heteroaryl selected from pyridinyl, imidazolyl, pyrimidinyl, pyrazolyl, triazolyl, pyrazinyl, tetrazolyl, furyl, thienyl, oxazolyl, isoxazolyl, thiazolyl, isothiazolyl, pyrrolyl, indolyl, benzimidazolyl, indazolyl, oxadiazolyl, and thiadiazolyl, wherein the substituents considered are one or more groups halogen, C₁-C₆-alkyl, halo-C₁-C₆-alkyl, hydroxy, C₁-C₆-alkoxy, optionally C₁-C₆-alkylated or C₁-C₂₀-acylated amino, pyrdiyl, aminopyridyl, or optionally substituted phenyl; and
R⁴ is hydrogen, methyl, a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, and 3-(dimethylamino)-1-propene-1-sulfonyl, a chain of 1 to 20 methylene groups either directly linked to X¹, X², E¹ or E², or linked to the reactive group, such chain that is substituted by oxo, C₁-C₆ alkyl, a further chain of 1 to 6 methylene groups, phenyl, phenylene, or residues of naturally occurring amino acids, or such optionally substituted chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally one further reactive group selected from acryloyl, methacryloyl, 4-dimethylamino-but-2-enoyl, and 4-(dimethylamino)-2,3-epoxy-butanoyl;
and tautomers, solvates and pharmaceutically acceptable salts thereof.

4. The compound according to claim 1 of formula (II) or (III) 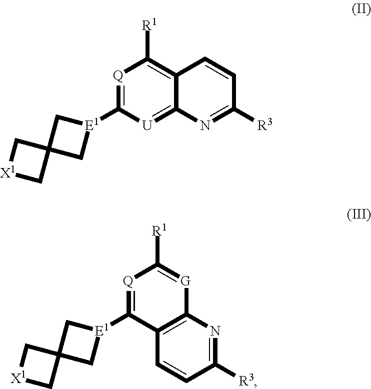
wherein
E¹ and E² are, independently of each other, N or N⁺R⁴;
X¹ and X² are, independently of each other, NR⁴ or O;
R¹ is (S)-2-methylmorpholino; (R)-2-methylmorpholino; 2-(aminocarbonylmethyl)-morpholino; 2-(benzamidomethyl)morpholino; (2R,6S)-2,6-dimethylmorpholino; (2R,6R)-2,6-dimethylmorpholino; (R)-3-methylmorpholino; (S)-3-methylmorpholino; (2R,3R)-2,3-dimethylmorpholino; (2S,5S)-2,5-dimethylmorpholino; (3S,5R)-3,5-dimethylmorpholino; (3S,5S)-3,5-dimethylmorpholino; octahydrocyclopenta[b][1,4]oxazin-4-yl; octahydro-2H-benzo[b][1,4]oxazin-4-yl; 3,4-dihydro-2H-benzo[b][1,4]oxazin-4-yl; 3-methoxycarbonylmethyl-2-methylmorpholino; 2-(methoxycarbonylmethyl)morpholino; 3-(methoxycarbonyl-methyl)morpholino; 2-vinylmorpholino; 2-(methoxycarbonylmethyl)-5-methylmorpholino; 3-(aminomethyl)morpholino; 2-(aminomethyl)morpholino; 2-cyanomorpholino; 2-(carboxy-methyl)morpholino; 3-(hydroxymethyl)morpholino; 2-(hydroxymethyl)morpholino; 2-(acetamidomethyl)morpholino; 2-(pyrrolidinocarbonylmethyl)morpholino; 2-(aminocarbonyl)-morpholino; 3-(aminocarbonyl)morpholino; 3-cyanomorpholino; 2,2,6,6-tetramethylmorpholino; 2,2,6-trimethylmorpholino; 8-oxa-3-azabicyclo[3.2.1]octan-3-yl; (1S,5R)-8-oxa-3-azabicyclo[3.2.1]octan-3-yl; (1R,55)-3-oxa-8-azabicyclo[3.2.1]octan-8-yl; piperidino, piperazino, 4-methylpiperazino; 4-(methoxycarbonyl)piperazino, 4-(methylsulfonyl)piperazino; or 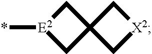
R³ is C₁-C₆-alkylamino, di-C₁-C₆-alkylamino, hydroxy-C₁-C₆-alkylamino, di(hydroxy-C₁-C₆-alkyl)amino, C₁-C₆-alkoxy-C₁-C₆-alkylamino, di(C₁-C₆-alkoxy-C₁-C₆-alkyl)amino, C₁-C₆-alkoxy-C₁-C₆-alkoxy-C₁-C₆-alkylamino, oxo-C₁-C₆-alkylamino, amino-C₁-C₆-alkylamino, C₁-C₆-alkylamino-C₁-C₆-alkylamino, di(C₁-C₆-alkyl)amino-C₁-C₆-alkylamino, hydroxy-C₁-C₆-alkylamino-C₁-C₆-alkylamino, di(hydroxy-C₁-C₆-alkyl)amino-C₁-C₆-alkylamino, C₁-C₆-alkylcarbonylamino-C₁-C₆-alkylamino, phenyl-C₁-C₆-alkylamino, C₂-C₆-alkenylamino, phenylamino, pyridylamino, pyrimidinylamino, pyrrolylamino, pyrrolidino, piperidino, piperazino, methylpiperazino, morpholino, dimethylmorpholino; phenyl or naphthyl, optionally substituted by one or more groups halogen, C₁-C₆-alkyl, halo-C₁-C₆-alkyl, hydroxy-C₁-C₆-alkyl, C₁-C₆-alkoxy-C₁-C₆-alkyl, oxo-C₁-C₆-alkyl, carboxy-C₁-C₆-alkyl, C₁-C₆-alkoxycarbonyl-C₁-C₆-alkyl, aminocarbonyl-C₁-C₆-alkyl, C₁-C₆-alkylaminocarbonyl-C₁-C₆-alkyl, amino-C₁-C₆-alkyl, C₁-C₆-alkylamino-C₁-C₆-alkyl, C₁-C₆-alkylcarbonylamino-C₁-C₆-alkyl, C₂-C₆-alkenylcarbonyl-amino-C₁-C₆-alkyl, phenyl-C₁-C₆-alkyl, C₂-C₆-alkenyl, C₂-C₆-alkynyl, hydroxy, C₁-C₆-alkoxy, C₁-C₆-alkoxy-C₁-C₆-alkoxy, amino, C₁-C₆-alkylamino, di-C₁-C₆-alkylamino, hydroxy-C₁-C₆-alkylamino, di(hydroxy-C₁-C₆-alkyl)amino, C₁-C₆-alkylcarbonylamino, halo-C₁-C₆-alkylcarbonylamino, C₂-C₆-alkenylcarbonylamino, C₁-C₆-alkyloxycarbonylamino, C₁-C₆-alkylaminocarbonylamino, pyridinylcarbonylamino, aminopyridinylcarbonylamino, amino-trifluoromethyl-pyridinylcarbonylamino, halo-C₁-C₆-alkylsulfonylamino, cyano, carboxy, C₁-C₆-alkoxycarbonyl, or aminocarbonyl; optionally substituted heteroaryl selected from pyridinyl, imidazolyl, pyrimidinyl, furyl, indolyl, benzimidazolyl, or indazolyl, wherein the substituents are selected from C₁-C₆-alkyl, halo-C₁-C₆-alkyl, amino or C₁-C₈-acylamino, wherein C₁-C₈-acyl is a C₁-C₇-alkyl, halo-C₁-C₇-alkyl, epoxy-C₁-C₇-alkyl, C₂-C₇-alkenyl, pyridyl or aminopyridyl group connected to carbonyl, oxycarbonyl or aminocarbonyl; and combinations thereof; and
R⁴ is hydrogen, methyl, a reactive group selected from acryloyl, methacryloyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-trichloroacetyl, methylsulfonyloxyacetyl, 2-chloropropionyl, 2,3-epoxypropionyl, (phenylthio)thiocarbonyl, 2-nitro-phenoxycarbonyl, 4-fluorophenoxycarbonyl, and 4-(3-(trifluoromethyl)-3H-diazirin-3-yl)benzamide, a chain of 1 to 20 optionally substituted methylene groups either directly linked to X¹, X², E¹ or E², or linked to the reactive group, or such chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, 3-(dimethylamino)-1-propene-1-sulfonyl, fluoro-, chloro-, bromo- or iodoacetyl, chloro- or bromomethanesulfonyl, 2,2-dichloroacetyl, 2,2,2-trichloroacetyl, methylsulfonyloxyacetyl, 2-chloropropionyl, 2,3-epoxypropionyl, (phenylthio)thiocarbonyl, 2-nitrophenoxycarbonyl, and 4-fluorophenoxycarbonyl;
and tautomers, solvates and pharmaceutically acceptable salts thereof.

5. The compound according to claim 1 of formula 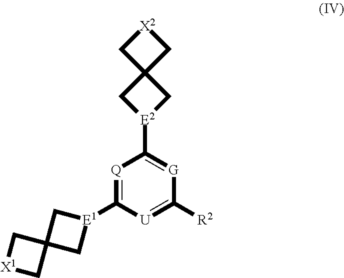
wherein
G is CH or N, Q is CH or N, and U is CH or N, with the proviso that at least two of G, Q and U are N, or one of G and U together with R² forms an anullated pyridine ring further substituted by R³, and the other one of G and U is N and Q is N;
E¹ and E² are, independently of each other, CR⁴, N, N⁺R⁴, or N→O;
X¹ and X² are, independently of each other, CHR⁴, CH₂CH₂, NR⁴, NR⁴→O, or O;
R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;
R³ is optionally substituted amino, optionally substituted C₆-C₂₀-aryl, or optionally substituted C₁-C₁₉-heteroaryl;
R⁴ is hydrogen, C₁-C₆-alkyl, C₁-C₆-acyl, C₁-C₆-acylamino-C₁-C₆-alkyl, a reactive group or a linker carrying a reactive group and/or a tag;
and tautomers, prodrugs, metabolites, solvates and pharmaceutically acceptable salts thereof.

6. The compound of formula (IV) according to claim 5, wherein
G is CH or N, Q is CH or N, and U is CH or N, with the proviso that at least two of G, Q and U are N;
E¹ and E² are, independently of each other, N or N⁺R⁴;
X¹ and X² are, independently of each other, NR⁴ or O;
R² is meta- or para-substituted phenyl or 2,4-, 3,4- or 3,5-disubstituted phenyl, wherein the substituents are selected from halogen, C₁-C₆-alkyl, halo-C₁-C₆-alkyl, hydroxy, C₁-C₆-alkoxy, optionally C₁-C₆-alkylated or C₁-C₂₀-acylated amino; or optionally substituted heteraryl selected from pyridinyl, imidazolyl, pyrimidinyl, furyl, indolyl, benzimidazolyl, indazolyl, oxadiazolyl, and thiadiazolyl, wherein the substituents are selected from C₁-C₆-alkyl, halo-C₁-C₆-alkyl, dimethoxyhydroxyphenyl, pyridyl, aminopyridyl, amino or C₁-C₈-acylamino, wherein C₁-C₈-acyl is a C₁-C₇-alkyl, halo-C₁-C₇-alkyl, epoxy-C₁-C₇-alkyl, C₂-C₇-alkenyl, pyridyl or aminopyridyl group connected to carbonyl, oxycarbonyl or aminocarbonyl; and combinations thereof; and
R⁴ is hydrogen, methyl, a reactive group selected from acryloyl, methacryloyl, 4-amino-but-2-enoyl, 4-dimethylamino-but-2-enoyl, 4-(dimethylamino)-2,3-epoxy-butanoyl, 3-amino-1-propene-1-sulfonyl, and 3-(dimethylamino)-1-propene-1-sulfonyl, a chain of 1 to 20 methylene groups either directly linked to X¹, X², E¹ or E², or linked to the reactive group, such chain that is substituted by oxo, C₁-C₆ alkyl, a further chain of 1 to 6 methylene groups, phenyl, phenylene, or residues of naturally occurring amino acids, or such optionally substituted chain wherein one or more methylene groups are replaced by oxygen, a carbonyloxy group, optionally substituted nitrogen, a carboxamide group, a urea group, sulphur, a disulfide group, or combinations thereof, carrying one or two tags selected from biotin, avidin, streptavidin, a fluorescent marker, a naturally occurring amino acid, and a solid phase, and optionally one further reactive group selected from acryloyl, methacryloyl, 4-dimethylamino-but-2-enoyl, and 4-(dimethylamino)-2,3-epoxy-butanoyl;
and tautomers, solvates and pharmaceutically acceptable salts thereof.

7. The compound according to claim 1 of formula 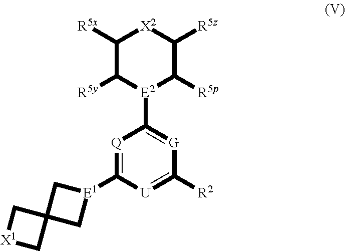
wherein
G is CH or N, Q is CH or N, and U is CH or N, with the proviso that at least two of G, Q and U are N, or one of G and U together with R² forms an anullated pyridine ring further substituted by R³, and the other one of G and U is N and Q is N;
E¹ and E² are, independently of each other, CR⁴, N, N⁺R⁴, or N→O;
X¹ and X² are, independently of each other, CHR⁴, CH₂CH₂, NR⁴, NR⁴→O, or O;
R² is hydrogen, halogen, cyano, nitro, C₁-C₆-alkyl, halo-C₁-C₆-alkyl, C₂-C₆-alkenyl, C₂-C₆-alkynyl, optionally substituted C₃-C₁₂-carbocyclyl, optionally substituted C₆-C₂₀-aryl, optionally substituted C₂-C₁₉-heterocyclyl, optionally substituted C₁-C₁₉-heteroaryl, C₁-C₆-alkylsulfonyl, halo-C₁-C₆-alkylsulfonyl, optionally substituted C₆-C₂₀-arylsulfonyl, optionally substituted aminosulfonyl, a reactive group, or a linker carrying a reactive group and/or a tag;
R³ is optionally substituted amino, optionally substituted C₆-C₂₀-aryl, or optionally substituted C₁-C₁₉-heteroaryl;
R⁴ is hydrogen, C₁-C₆-alkyl, C₁-C₆-acyl, C₁-C₆-acylamino-C₁-C₆-alkyl, a reactive group or a linker carrying a reactive group and/or a tag;
R^{5x}, R^{5y}, R^{5z} and R^{5p} are, independently of each other, hydrogen, halogen, cyano, optionally substituted C₁-C₆ alkyl, C₂-C₆ alkenyl, or C₂-C₆ alkynyl, or one or two of R^{5x}, R^{5y}, R^{5z} and R^{5p} are two geminal substituents methyl and the other ones are hydrogen, or R^{5x} and R^{5y}, or R^{5z} and R^{5p} form together an anullated five- or six-membered carbocyclyl, heterocyclyl, aryl or heteroaryl ring, or R^{5x} and R^{5p} form together bridging ethylene, or R^{5y} and R^{5p} form together bridging ethylene;
and tautomers, prodrugs, metabolites, solvates and pharmaceutically acceptable salts thereof.

8. The compound according to claim 1 selected from the group consisting of 6-amino-N-(3-(4-morpholino-6-(2-oxa-6-azaspiro[3.3]heptan-6-yl)-1,3,5-triazin-2-yl)phenyl)nicotinamide (example 111); N-(3-(4-morpholino-6-(2-oxa-6-azaspiro[3.3]heptan-6-yl)-1,3,5-triazin-2-yl)phenyl)nicotinamide (125); methyl (4-(4-morpholino-6-(2-oxa-6-azaspiro[3.3]heptan-6-yl)-1,3,5-triazin-2-yl)phenyl)carbamate (131); methyl (4-(4-(8-oxa-3-azabicyclo[3.2.1]octan-3-yl)-6-(2-oxa-6-azaspiro[3.3]heptan-6-yl)-1,3,5-triazin-2-yl)phenyl)carbamate (132); 1-methyl-3-(4-(4-morpholino-6-(2-oxa-6-azaspiro[3.3]heptan-6-yl)-1,3,5-triazin-2-yl)phenyl)urea (141); 1-(4-(4-(8-oxa-3-azabicyclo[3.2.1]octan-3-yl)-6-(2-oxa-6-azaspiro[3.3]heptan-6-yl)-1,3,5-triazin-2-yl)phenyl)-3-methylurea (146); 1-ethyl-3-(4-(4-morpholino-6-(2-oxa-6-azaspiro[3.3]heptan-6-yl)-1,3,5-triazin-2-yl)phenyl)urea (151); 1-(4-(4-(8-oxa-3-azabicyclo[3.2.1]octan-3-yl)-6-(2-oxa-6-azaspiro[3.3]heptan-6-yl)-1,3,5-triazin-2-yl)-phenyl)-3-ethylurea (155); 1-ethyl-3-(5-(4-morpholino-6-(2-oxa-6-azaspiro[3.3]heptan-6-yl)-1,3,5-triazin-2-yl)pyridin-2-yl)urea (160); 1-(5-(4-(8-oxa-3-azabicyclo[3.2.1]octan-3-yl)-6-(2-oxa-6-azaspiro[3.3]heptan-6-yl)-1,3,5-triazin-2-yl)pyridin-2-yl)-3-ethylurea (164); 1-ethyl-3-(5-(4-morpholino-6-(2-oxa-6-azaspiro[3.3]heptan-6-yl)-1,3,5-triazin-2-yl)pyrimidin-2-yl)urea (169); 1-(5-(4-(8-oxa-3-azabicyclo[3.2.1]octan-3-yl)-6-(2-oxa-6-azaspiro[3.3]heptan-6-yl)-1,3,5-triazin-2-yl)pyrimidin-2-yl)-3-ethylurea (173); 1-(4-(4-(dimethylamino)piperidine-1-carbonyl)phenyl)-3-(4-(4-morpholino-6-(2-oxa-6-azaspiro[3.3]heptan-6-yl)-1,3,5-triazin-2-yl)phenyl)urea (196); 1-(4-(4-(dimethylamino)piperidine-1-carbonyl)phenyl)-3-(5-(4-morpholino-6-(2-oxa-6-azaspiro[3.3]heptan-6-yl)-1,3,5-triazin-2-yl)pyridin-2-yl)urea (200); 1-(4-(4-(dimethylamino)piperidine-1-carbonyl)phenyl)-3-(5-(4-morpholino-6-(2-oxa-6-azaspiro[3.3]heptan-6-yl)-1,3,5-triazin-2-yl)pyrimidin-2-yl)urea (204); 5-(4-morpholino-6-(2-oxa-6-azaspiro[3.3]heptan-6-yl)-1,3,5-triazin-2-yl)pyridin-2-amine (215); 4-methyl-5-(4-morpholino-6-(2-oxa-6-azaspiro[3.3]heptan-6-yl)-1,3,5-triazin-2-yl)pyridin-2-amine (220); 5-(4-(8-oxa-3-azabicyclo[3.2.1]octan-3-yl)-6-(2-oxa-6-azaspiro[3.3]heptan-6-yl)-1,3,5-triazin-2-yl)-4-methylpyridin-2-amine (221); 5-(4-morpholino-6-(2-oxa-6-azaspiro[3.3]heptan-6-yl)-1,3,5-triazin-2-yl)-4-(trifluoromethyl)pyridin-2-amine (225); 5-(4-morpholino-6-(2-oxa-6-azaspiro[3.3]heptan-6-yl)pyrimidin-2-yl)-4-(trifluoromethyl)pyridin-2-amine (227); 5-(6-morpholino-2-(2-oxa-6-azaspiro[3.3]heptan-6-yl)pyrimidin-4-yl)-4-(trifluoromethyl)pyridin-2-amine (228); 5-(2-morpholino-6-(2-oxa-6-azaspiro[3.3]heptan-6-yl)pyrimidin-4-yl)-4-(trifluoromethyl)pyridin-2-amine (229); 5-(4-(6-methyl-2,6-diazaspiro[3.3]heptan-2-yl)-6-morpholino-1,3,5-triazin-2-yl)-4-(trifluoromethyl)pyridin-2-amine (231); 5-(4-morpholino-6-(2-oxa-6-azaspiro[3.3]heptan-6-yl)-1,3,5-triazin-2-yl)pyrimidin-2-amine (251); 5-(4-(8-oxa-3-azabicyclo[3.2.1]octan-3-yl)-6-(2-oxa-6-azaspiro[3.3]heptan-6-yl)-1,3,5-triazin-2-yl)pyrimidin-2-amine (252); 4-morpholino-6-(2-oxa-6-azaspiro[3.3]heptan-6-yl)-[2,5′-bipyrimidin]-2′-amine (253); 6-morpholino-2-(2-oxa-6-azaspiro[3.3]heptan-6-yl)-[4,5′-bipyrimidin]-2′-amine (254); 2-morpholino-6-(2-oxa-6-azaspiro[3.3]heptan-6-yl)-[4,5′-bipyrimidin]-2′-amine (255); 5-(4-(6-methyl-2,6-diazaspiro[3.3]heptan-2-yl)-6-morpholino-1,3,5-triazin-2-yl)pyrimidin-2-amine (257); 1-(6-(4-(2-aminopyrimidin-5-yl)-6-morpholino-1,3,5-triazin-2-yl)-2,6-diazaspiro[3.3]-heptan-2-yl)prop-2-en-1-one (259); 1-(6-(4-(2-aminopyrimidin-5-yl)-6-morpholino-1,3,5-triazin-2-yl)-2,6-diazaspiro[3.3]heptan-2-yl)-2-chloroethanone (261); 4-methyl-5-(4-morpholino-6-(2-oxa-6-azaspiro[3.3]heptan-6-yl)-1,3,5-triazin-2-yl)pyrimidin-2-amine (268); 5-(4-(8-oxa-3-azabicyclo[3.2.1]octan-3-yl)-6-(2-oxa-6-azaspiro[3.3]heptan-6-yl)-1,3,5-triazin-2-yl)-4-methylpyrimidin-2-amine (269); 5-(4-morpholino-6-(2-oxa-6-azaspiro[3.3]heptan-6-yl)-1,3,5-triazin-2-yl)-4-(trifluoromethyl)pyrimidin-2-amine (273); 4-morpholino-6-(2-oxa-6-azaspiro[3.3]heptan-6-yl)-4′-(trifluoromethyl)-[2,5′-bipyrimidin]-2′-amine (276); 6-morpholino-2-(2-oxa-6-azaspiro[3.3]heptan-6-yl)-4′-(trifluoromethyl)-[4,5′-bipyrimidin]-2′-amine (277); 2-morpholino-6-(2-oxa-6-azaspiro[3.3]heptan-6-yl)-4′-(trifluoromethyl)-[4,5′-bipyrimidin]-2′-amine (278); 6-(4-(2-(difluoromethyl)-1H-benzo[d]imidazol-1-yl)-6-morpholino-1,3,5-triazin-2-yl)-2-oxa-6-azaspiro[3.3]heptane (299); 6-(2-(2-(difluoromethyl)-1H-benzo[d]imidazol-1-yl)-6-morpholinopyrimidin-4-yl)-2-oxa-6-azaspiro[3.3]heptane (300); 6-(4-(2-(difluoromethyl)-1H-benzo[d]imidazol-1-yl)-6-morpholinopyrimidin-2-yl)-2-oxa-6-azaspiro[3.3]heptane (301); 6-(6-(2-(difluoromethyl)-1H-benzo[d]imidazol-1-yl)-2-morpholinopyrimidin-4-yl)-2-oxa-6-azaspiro[3.3]heptane (302); 4-(4-(2-(difluoromethyl)-1H-benzo[d]imidazol-1-yl)-6-(2,6-diazaspiro[3.3]heptan-2-yl)-1,3,5-triazin-2-yl)morpholine (303); 4-(4-(2-(difluoromethyl)-1H-benzo[d]imidazol-1-yl)-6-(6-methyl-2,6-diazaspiro[3.3]heptan-2-yl)-1,3,5-triazin-2-yl)morpholine (304); 3-(4-(2-(difluoromethyl)-1H-benzo[d]imidazol-1-yl)-6-(2-oxa-6-azaspiro[3.3]heptan-6-yl)-1,3,5-triazin-2-yl)-8-oxa-3-azabicyclo[3.2.1]octane (308); N-(2-(6-(4-(2-(difluoromethyl)-1H-benzo[d]imidazol-1-yl)-6-morpholino-1,3,5-triazin-2-yl)-2,6-diazaspiro[3.3]heptan-2-yl)ethyl)acrylamide (312); 2-chloro-N-(2-(6-(4-(2-(difluoromethyl)-1H-benzo[d]imidazol-1-yl)-6-morpholino-1,3,5-triazin-2-yl)-2,6-diazaspiro[3.3]heptan-2-yl)ethyl)acetamide (316); 1-(6-(4-(2-(difluoromethyl)-1H-benzo[d]imidazol-1-yl)-6-morpholino-1,3,5-triazin-2-yl)-2,6-diazaspiro[3.3]heptan-2-yl)prop-2-en-1-one (317); 2-chloro-1-(6-(4-(2-(difluoromethyl)-1H-benzo[d]imidazol-1-yl)-6-morpholino-1,3,5-triazin-2-yl)-2,6-diazaspiro-[3.3]heptan-2-yl)ethanone (323); 2,6-dimethoxy-4-(1-(4-morpholino-6-(2-oxa-6-azaspiro-[3.3]heptan-6-yl)-1,3,5-triazin-2-yl)-1H-imidazol-4-yl)phenol (345); 4-(1-(4-(8-oxa-3-aza-bicyclo [3.2.1]octan-3-yl)-6-(2-oxa-6-azaspiro[3.3]heptan-6-yl)-1,3,5-triazin-2-yl)-1H-imidazol-4-yl)-2,6-dimethoxyphenol (346); 2,6-dimethoxy-4-(5-(4-morpholino-6-(2-oxa-6-azaspiro[3.3]heptan-6-yl)-1,3,5-triazin-2-yl)furan-2-yl)phenol (351); 4-(5-(4-(8-oxa-3-azabicyclo[3.2.1]octan-3-yl)-6-(2-oxa-6-azaspiro[3.3]heptan-6-yl)-1,3,5-triazin-2-yl)furan-2-yl)-2,6-dimethoxyphenol (352); (E)-3-((6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)prop-2-en-1-amine (366); (E)-3-((6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)-N,N-dimethylprop-2-en-1-amine (367); (E)-1-(6-(4-(2-(difluoromethyl)-1H-benzo[d]imidazol-1-yl)-6-morpholino-1,3,5-triazin-2-yl)-2,6-diazaspiro[3.3]heptan-2-yl)-4-(dimethylamino)but-2-en-1-one (368); N-((E)-3-((6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)allyl)-5-((3aS,4S,6aR)-2-oxohexahydro-1H-thieno[3,4-d]imidazol-4-yl)pentanamide (369); N-((E)-4-(6-(4-(2-(difluoromethyl)-1H-benzo[d]imidazol-1-yl)-6-morpholino-1,3,5-triazin-2-yl)-2,6-diazaspiro[3.3]heptan-2-yl)-4-oxobut-2-en-1-yl)-5-((3aS,4S,6aR)-2-oxohexahydro-1H-thieno[3,4-d]imidazol-4-yl)pentanamide (370); (E)-3-(4-(2-((6-(6-(4-(2-(difluoromethyl)-1H-benzo[d]imidazol-1-yl)-6-morpholino-1,3,5-triazin-2-yl)-2,6-diazaspiro[3.3]heptan-2-yl)-6-oxohexyl)amino)-2-oxoethoxy)styryl)-5,5-difluoro-7-(thiophen-2-yl)-5H-dipyrrolo[1,2-c:2′,1′-f][1,3,2]diazaborinin-4-ium-5-uide (371); (5-(4-(8-oxa-3-aza-bicyclo[3.2.1]octan-3-yl)-2-(2-oxa-6-azaspiro[3.3]heptan-6-yl)pyrido[2,3-d]pyrimidin-7-yl)-2-methoxyphenyl)methanol (372); (5-(4-((3R,5S)-3,5-dimethylmorpholino)-2-(2-oxa-6-azaspiro[3.3]heptan-6-yl)pyrido[2,3-d]pyrimidin-7-yl)-2-methoxyphenyl)methanol (374); and (2-methoxy-5-(4-morpholino-2-(2-oxa-6-azaspiro[3.3]heptan-6-yl)pyrido[2,3-d]pyrimidin-7-yl)phenyl)methanol (375).

9. The compound according to claim 1 selected from the group consisting of methyl (4-(4-morpholino-6-(2-oxa-6-azaspiro[3.3]heptan-6-yl)-1,3,5-triazin-2-yl)phenyl)carbamate (131); methyl (4-(4-(8-oxa-3-azabicyclo[3.2.1]octan-3-yl)-6-(2-oxa-6-azaspiro[3.3]heptan-6-yl)-1,3,5-triazin-2-yl)phenyl)carbamate (132); 1-methyl-3-(4-(4-morpholino-6-(2-oxa-6-azaspiro[3.3]heptan-6-yl)-1,3,5-triazin-2-yl)phenyl)urea (141); 1-(4-(4-(8-oxa-3-azabicyclo[3.2.1]octan-3-yl)-6-(2-oxa-6-azaspiro[3.3]heptan-6-yl)-1,3,5-triazin-2-yl)phenyl)-3-methylurea (146); 5-(4-morpholino-6-(2-oxa-6-azaspiro[3.3]heptan-6-yl)-1,3,5-triazin-2-yl)pyridin-2-amine (215); 4-methyl-5-(4-morpholino-6-(2-oxa-6-azaspiro[3.3]heptan-6-yl)-1,3,5-triazin-2-yl)pyridin-2-amine (220); 4-morpholino-6-(2-oxa-6-azaspiro[3.3]heptan-6-yl)-[2,5′-bipyrimidin]-2′-amine (253); 6-morpholino-2-(2-oxa-6-azaspiro[3.3]heptan-6-yl)-[4,5′-bipyrimidin]-2′-amine (254); 2-morpholino-6-(2-oxa-6-azaspiro[3.3]heptan-6-yl)-[4,5′-bipyrimidin]-2′-amine (255); 5-(4-(6-methyl-2,6-diazaspiro[3.3]heptan-2-yl)-6-morpholino-1,3,5-triazin-2-yl)pyrimidin-2-amine (257); 1-(6-(4-(2-aminopyrimidin-5-yl)-6-morpholino-1,3,5-triazin-2-yl)-2,6-diazaspiro[3.3]heptan-2-yl)prop-2-en-1-one (259); 1-(6-(4-(2-aminopyrimidin-5-yl)-6-morpholino-1,3,5-triazin-2-yl)-2,6-diazaspiro[3.3]heptan-2-yl)-2-chloroethanone (261); 4-methyl-5-(4-morpholino-6-(2-oxa-6-azaspiro[3.3]heptan-6-yl)-1,3,5-triazin-2-yl)pyrimidin-2-amine (268); 4-(4-(2-(difluoromethyl)-1H-benzo[d]imidazol-1-yl)-6-(6-methyl-2,6-diazaspiro[3.3]heptan-2-yl)-1,3,5-triazin-2-yl)morpholine (304); 2,6-dimethoxy-4-(5-(4-morpholino-6-(2-oxa-6-azaspiro[3.3]heptan-6-yl)-1,3,5-triazin-2-yl)furan-2-yl)phenol (351); (E)-3-(6-(4-(2-(difluoromethyl)-1H-benzo[d]imidazol-1-yl)-6-morpholino-1,3,5-triazin-2-yl)-2,6-diazaspiro[3.3]heptan-2-yl)sulfonyl)-N,N-dimethylprop-2-en-1-amine (367); (5-(4-(8-oxa-3-azabicyclo[3.2.1]octan-3-yl)-2-(2-oxa-6-azaspiro[3.3]heptan-6-yl)pyrido[2,3-d]pyrimidin-7-yl)-2-methoxyphenyl)methanol (372); (5-(4-((3R,5S)-3,5-dimethylmorpholino)-2-(2-oxa-6-azaspiro[3.3]heptan-6-yl)pyrido[2,3-d]pyrimidin-7-yl)-2-methoxyphenyl)methanol (374); and (2-methoxy-5-(4-morpholino-2-(2-oxa-6-azaspiro[3.3]heptan-6-yl)pyrido[2,3-d]pyrimidin-7-yl)phenyl)methanol (375).

10. The compound according to claim 1 selected from the group consisting of 1-ethyl-3-(4-(4-morpholino-6-(2-oxa-6-azaspiro[3.3]heptan-6-yl)-1,3,5-triazin-2-yl)phenyl)urea (151); 1-(4-(4-(8-oxa-3-azabicyclo[3.2.1]octan-3-yl)-6-(2-oxa-6-azaspiro[3.3]heptan-6-yl)-1,3,5-triazin-2-yl)phenyl)-3-ethylurea (155); 1-ethyl-3-(5-(4-morpholino-6-(2-oxa-6-azaspiro[3.3]heptan-6-yl)-1,3,5-triazin-2-yl)pyridin-2-yl)urea (160); 1-(5-(4-(8-oxa-3-azabicyclo[3.2.1]octan-3-yl)-6-(2-oxa-6-azaspiro[3.3]heptan-6-yl)-1,3,5-triazin-2-yl)pyridin-2-yl)-3-ethylurea (164); 1-ethyl-3-(5-(4-morpholino-6-(2-oxa-6-azaspiro[3.3]heptan-6-yl)-1,3,5-triazin-2-yl)pyrimidin-2-yl)urea (169); 1-(5-(4-(8-oxa-3-azabicyclo[3.2.1]octan-3-yl)-6-(2-oxa-6-azaspiro[3.3]heptan-6-yl)-1,3,5-triazin-2-yl)pyrimidin-2-yl)-3-ethylurea (173); 1-(4-(4-(dimethylamino)piperidine-1-carbonyl)phenyl)-3-(4-(4-morpholino-6-(2-oxa-6-azaspiro[3.3]heptan-6-yl)-1,3,5-triazin-2-yl)phenyl)urea (196); 1-(4-(4-(dimethylamino)piperidine-1-carbonyl)phenyl)-3-(5-(4-morpholino-6-(2-oxa-6-azaspiro[3.3]heptan-6-yl)-1,3,5-triazin-2-yl)pyridin-2-yl)urea (200); 1-(4-(4-(dimethyl-amino)piperidine-1-carbonyl)phenyl)-3-(5-(4-morpholino-6-(2-oxa-6-azaspiro[3.3]heptan-6-yl)-1,3,5-triazin-2-yl)pyrimidin-2-yl)urea (204); 5-(4-morpholino-6-(2-oxa-6-azaspiro[3.3]heptan-6-yl)-1,3,5-triazin-2-yl)-4-(trifluoromethyl)pyridin-2-amine (225); 5-(4-morpholino-6-(2-oxa-6-azaspiro[3.3]heptan-6-yl)pyrimidin-2-yl)-4-(trifluoromethyl)pyridin-2-amine (227); 5-(6-morpholino-2-(2-oxa-6-azaspiro[3.3]heptan-6-yl)pyrimidin-4-yl)-4-(trifluoromethyl)pyridin-2-amine (228); 5-(2-morpholino-6-(2-oxa-6-azaspiro[3.3]heptan-6-yl)pyrimidin-4-yl)-4-(trifluoromethyl)pyridin-2-amine (229); 5-(4-morpholino-6-(2-oxa-6-azaspiro[3.3]heptan-6-yl)-1,3,5-triazin-2-yl)pyrimidin-2-amine (251); 5-(4-(8-oxa-3-azabicyclo[3.2.1]octan-3-yl)-6-(2-oxa-6-azaspiro[3.3]heptan-6-yl)-1,3,5-triazin-2-yl)pyrimidin-2-amine (252); 5-(4-morpholino-6-(2-oxa-6-azaspiro[3.3]heptan-6-yl)-1,3,5-triazin-2-yl)-4-(trifluoromethyl)pyrimidin-2-amine (273); 4-morpholino-6-(2-oxa-6-azaspiro[3.3]heptan-6-yl)-4′-(trifluoromethyl)-[2,5′-bipyrimidin]-2′-amine (276); 6-morpholino-2-(2-oxa-6-azaspiro[3.3]heptan-6-yl)-4′-(trifluoromethyl)-[4,5′-bipyrimidin]-2′-amine (277); 2-morpholino-6-(2-oxa-6-azaspiro[3.3]heptan-6-yl)-4′-(tri-fluoromethyl)-[4,5′-bipyrimidin]-2′-amine (278); 6-(4-(2-(difluoromethyl)-1H-benzo[d]imidazol-1-yl)-6-morpholino-1,3,5-triazin-2-yl)-2-oxa-6-azaspiro[3.3]heptane (299); 6-(2-(2-(difluoromethyl)-1H-benzo[d]imidazol-1-yl)-6-morpholinopyrimidin-4-yl)-2-oxa-6-azaspiro-[3.3]heptane (300); 6-(4-(2-(difluoromethyl)-1H-benzo[d]imidazol-1-yl)-6-morpholino-pyrimidin-2-yl)-2-oxa-6-azaspiro[3.3]heptane (301); 6-(6-(2-(difluoromethyl)-1H-benzo-[d]imidazol-1-yl)-2-morpholinopyrimidin-4-yl)-2-oxa-6-azaspiro[3.3]heptane (302); 4-(4-(2-(difluoromethyl)-1H-benzo[d]imidazol-1-yl)-6-(2,6-diazaspiro[3.3]heptan-2-yl)-1,3,5-triazin-2-yl)morpholine (303); N-(2-(6-(4-(2-(difluoromethyl)-1H-benzo[d]imidazol-1-yl)-6-morpholino-1,3,5-triazin-2-yl)-2,6-diazaspiro[3.3]heptan-2-yl)ethyl)acrylamide (312); 2-chloro-N-(2-(6-(4-(2-(difluoromethyl)-1H-benzo[d]imidazol-1-yl)-6-morpholino-1,3,5-triazin-2-yl)-2,6-diazaspiro-[3.3]heptan-2-yl)ethyl)acetamide (316); 1-(6-(4-(2-(difluoromethyl)-1H-benzo[d]imidazol-1-yl)-6-morpholino-1,3,5-triazin-2-yl)-2,6-diazaspiro[3.3]heptan-2-yl)prop-2-en-1-one (317); 2-chloro-1-(6-(4-(2-(difluoromethyl)-1H-benzo[d]imidazol-1-yl)-6-morpholino-1,3,5-triazin-2-yl)-2,6-diazaspiro[3.3]heptan-2-yl)ethanone (323); 4-(5-(4-(8-oxa-3-azabicyclo[3.2.1]octan-3-yl)-6-(2-oxa-6-azaspiro[3.3]heptan-6-yl)-1,3,5-triazin-2-yl)furan-2-yl)-2,6-dimethoxyphenol (352); (E)-1-(6-(4-(2-(difluoromethyl)-1H-benzo[d]imidazol-1-yl)-6-morpholino-1,3,5-triazin-2-yl)-2,6-diazaspiro[3.3]heptan-2-yl)-4-(dimethylamino)but-2-en-1-one (368); and (E)-3-(4-(2-(6-(6-(4-(2-(difluoromethyl)-1H-benzo[d]imidazol-1-yl)-6-morpholino-1,3,5-triazin-2-yl)-2,6-diaza-spiro[3.3]heptan-2-yl)-6-oxohexyl)amino)-2-oxoethoxy)styryl)-5,5-difluoro-7-(thiophen-2-yl)-5H-dipyrrolo[1,2-c:2′,1′-f][1,3,2]diazaborinin-4-ium-5-uide (371).

11. A pharmaceutical composition comprising a compound of formula (I) as claimed in claim 1 and a pharmaceutically acceptable carrier.

12. A method of inhibiting PI3 kinase activity, comprising contacting a PI3 kinase with an effective inhibitory amount of a compound of formula (I) as claimed in claim 1.

13. A method of preventing or treating a disease or disorder modulated by PI3 kinases and/or mTOR, comprising administering to a mammal in need of such treatment an effective amount of a compound of formula (I) as claimed in claim 1.

14. A method of preventing or treating a hyperproliferative disorder, comprising administering to a mammal in need of such treatment an effective amount of a compound of formula (I) as claimed in claim 1, alone or in combination with one or more additional compounds having anti-hyperproliferative properties.
